SEQUENCE LISTING (75 sequences)

SEQ ID NO: 1 - DNA, 27689 bp, Arabidopsis thaliana
gaattcggtgaaattttgccaaatttatactatagactatagagtgtgaattattttatgtacttttggtatgaagataagaattttggtattatcacaatcatcatgaaaataaaggctaactaacttcattgattatagttgtttatttggataagttatttttgggaggagaaattaattggaacaatttaatagacattgacagggtattgtattattatacatatacagatgcatttataatcctgccaactcttgaatgtgcattagatttcatttcaggtcatctttttttttgtttggtaaggccactaatattaagttaaccaatcataatttcatattatggattttaattggaatctagcgttttttttttttttccatttgttagactatttacttttctttataatttttttttgtgatttttattcaagcgaagtagggttttcaatttttaattctgtattttacattgttctaggacataaaacatcgcaacaaaatagaagttgttattttcagcagtttctaacgagttcaaattttatttagttgatagtttgataccggtttcttatcaattacaaatctataaccaatgcaaaaaattttatatggaaatgtaaacgatgacactaattaaaaccaataaattatcgggaccctaaattataagtttcaagttattctgctgatatcgtttaaaaaagcaaacttaaatggtatggacactctcaggtccgacggtattaatattgccttataacattatggtccatataataatatacatgattaccccactattaacaatagataacaacaaatttatcaattgcgatctctgaagactataactttgatattcgtattaacttgtatatacacagaacagaagcaataaaacttggaacatgtcacagaaattcaaactattttgttctctactattggttttaggtttcaagagagaagtatagagaaaatgtgatgtaataataatcggagatcgacgtaagatgttcgtggtgggtaaaggtgggacccataaaagagtctggtggtccagctagataaataattaatacttactaataaattaaataatggagaaataaatagtgtaaatagtaaagaagtaggtttgttccttccttaattaacgctattgccatttggatttgtggtggggacgactctctttcgatgcttcctcgacaaataaagctgtgccttctctatgttcacttcaagggtaaaaacgtcattatatcactattttttttttttttttcaagttccactacgacacgtcagtaattcaattcgttaatggttattggtcaaacgacgttaacttctctcacgtgagattaaacacaacaaataactgctacttgcaaggttttgacaatttaaaacaatcttagcctacgttacaggcgcaaaagattcattcaaatgtttaatttgaggtttagtgtaattaatttaatgtaaactaaaagcaatacaacgtgagactgaatgaaactaatgattatgttgtatttttataattcgtatgatgatgaatatgaaacttgttaaattgttgcttgttccgtccgacacattgagaagccaattcaaaatttgcagctagctatcattttttacccacttatatattcgtattattgatacttcaaatgcatgcacacgtatagtggtaaacatattcattatgtatccctctctataagatttttatgtgttgatgaaatttttatgttccctattttattgtaatcagtttgaatatattttcgtttttttgaatagctcatcaaaccgaaacccttcacatgatcacctaaaaatagtaaattttcaagatcatgcattcgaacaccattgataagagaaaactcattggttgtcaaaatactatctataaatccatatttaatatttcaatgttagaggtttcagtttcagtttgtgactgtgttattaactatcatctatatacgataaatgcagaaaaataatgtttgcatccatgatatcttgtaagtgtgtggcaaaagaataaaatattttgattttatttaggaataaatgtagataaatatagtatttttgatatgtaaacttgtataaaagttgggaaagttgtggttgataaaatctatccactacaaaaaaaaaaaaaaaaaacatttttgacgtcagttttgaaatactttaacgtcggtactaaagtgatcctaataatataacgtcagttttgaaattaatcgactaaatttgtgatagcaattaaaaacaatcctaagccaaggttgtgtgtaatacgtgagtaaatcatatactcaaggtgactaaattagtggcacgatcgaacttaacatcctatgaacctatagtttgtttctttgataaacagatcacttatagcatagaaatatagaagaaaataaagagaagaaacaaaattgtaacaagatccaagaattttgaatagacttttactctttatttaattttgtgccttctcattaaaagaagggaagaataaatttaatcgacattcccactaaagaatccaaagtcaacttctcaaatcttatggtacaggaaaaattatatgtaaagtgatgtataaattatgatttatcgcatggtgcaccgcaatcatcaaaaaaaaatatggccaccctttttattatcttcttccacgtttccgctatactcttacatttgtcttggtctttattttgttttcaatgccaaaaaaactttcactagcttcataatcttttttttttgttgaacaaagagcttcataatcttgaattcttgatcatgttttcttcaatagaaccaatacatagaaaaataatctttcttgctctcattttagccacaacattgatttcaacgttattattttgcttacaacatcatcacatacataatactctaattttcattagatatacttaaccattataaatttcacataacaagtcgagatattaattaatctaggcttcatttacggaagaaaaaaaatcgataaagaaagcaacaacatgatactcatatactcctaatgatgacaatcatggagcttaggaaaataacacatctctacttggtgtttcaactttttatgtttttgccatttttacccctgtaccattgccgccaaaatcagaaagtgctctggggccaataatactatatggtcaaatggactcaacgtacctttctccctttttctttttgatagcgattgcgccaatataaaaagggtatttccagtggtggttctgtcaattagacaagatctctttctcaatctccaagataagaaaaagttaagattatgatcccaatgttatttgaaggttaacacttaacactatagtttggttttagagtttagtcaagttactaaacacttcattgttcaatgtttctattcccaaactccaacacacggtatcttacatagaggtcatttgggacaccacgatttggtccccttcaagaaccttatttcatttccaatattgattattttcattgttcaatgtctcatatttttctgaaagaaaatgtatagctgattagtcataatcgtagcaaacttcttgattttgttatatgcttagttagtcgttaatcgtctattcttcggctagcatatttttcgggattaatttagccattagtccaaattattcagacaacacgttcttgaaaactaaaccttattaatataatcacgaatgctttggattcaatgtacttagtgaaccatataacatttgactgtagaaatttatagcaatttactttatcaatcctcccaaatcactttatcaaagtaatggggtcaaatgttaaaaattgaaaagttatggagtgaaaccttaaattacataaatgctcttccaaggggaagaagactaagagtatagaatctctctaaccagaggttctatcaccaccaaaacctgattgtaagaagccggtttcctccagatgactcacatcaccccaaacttggatggtccatttgtctccgtcgaataaacggaacacattgaccgatgtattgagtatcttttcaacttttcttgcacttggacgagctctttcgtagagagatcggatcacacctccatgagtcactactactatcctttcaccttcatcaaccaccaagtagacagaaaccaatacttagaacatatgtgcagtgtagtaaactgtgagaaagaaacacacagagaagaggaagaaacctttatgtttgtcgccgattctctgtaatgcagttgtacatctatcgtaaagtttgtcaagactttctcctccacccttcattcacataaacgaagcaaaagttagttgaatacagaaaccaagcatcattcatataactaagcttcttatcttactggaatatcaacgtctgtgcggttagatgaaaaagcctagtaagcttccgggcgaattttcgaagcttcttgatacacaagcccttgcatatctcctaaatgtctttcccgcaaatcacgatcggtaagcacctagacaaccattattcgtagttaaacttctatgaagtctagaaagtcatatggattcagcttcaaaacttagatccctctactaaaaatcaagcacaaaggtgaagactttagtgggtttgacacatagcattatgtctttagatgtctacctcaagcttgccgcatttagcagcaatgatctgagcagtctcaaaggctctcttcaagtcagaagagtatacatgagatatcttctgctccttcgataaccgctctgcaaccttcatttcaaatgaaagaaatgttttaaaaggcatcatgatgattaaggttacaacacaatcaaaagataacacatactctttgtgcttgttgtcttcctgcatcatttaactcaacatccaaatgaccctgtaacaccaaattataaccacatttcaacatctctagtcaaaattcccaataactatacattcacaaagatgctaatcattactaaatacaagataaagattcaaaatttacgatttctaagtcaagtttcgattccaaagatacagatacaaactccattattgttaaatttacctggatttttctctcggcattccaagatgtttcaccatgacgaacaacaacaatctcagcataatcaacatcttcagaactgtagtaaaaccaacaaagggatttcgtcaaaaacacgatcaaagatgcaaataacgagaagaagctagaagaagaagcatactcgaatcttgattcctccatgggagatgctgaaatctgacaattgctttagattcatccaacgacgagaaaccaattccattgatatgacattaacactaactgggctttacttagattttgtttatgggccttgccctttgttcgaacgaagcaaatatgttttcgttttttttcctctctcacgttaaaatcaataacaaactttattacatactactacagtacaacttctataaattaatactcgttaaattaataatctctataaattaattaattattccagtcccgaattgaaatcaatataaaaattgatataactcgataaattaataagattctaatttttaaaaattcttatataaatatatggtccaatcaatagtataaattaataataatataaatttaccaatatatattataaatatcatatgtaaaatatgacattgtttttcttcaatttttattattttttttgtatgttcaattctaatattttacaacataaaaaaaaattgtattgtttttaaacttaataattaatttgtatttttaattcgtttcataaatattattgattataaaaaaaaaaacttcattttatatcaaatttttgtaagaatttacaaaatttgtacaattttaattaatttatcgataacttaatatctctctaaattaataaatttttttggtctcaatattattaatttatagaggttttactgtagttggttaatttcatgaaaacatgtatggagaaataaaatagtaacataacaaaaacttgctactgcatccgccatatctggatctctaactgtagctgaagagaggccaaagcaaatacgtaaaactctgctaaatttcttataaaatatttggaacgaagcaaataatatttaattattacaatgatatttagctaactatatattaacctgcggtataccgcggtaggcttatattttagttaaactaaaactttttattgtaaagatgatttataaatagataattttattttatttgattttaaatgtatagtaaactgcgagttgtatatgttttgttgatattatttatattgtttagtgtttaagattatacacttgtattttaattgttaattttagagtttcacctgtagtataccatcttctattaatatcgatcttaacccgtcaattctaggattttccagcttgtattaaaaattgaatcacatcatacacataaaaaaatctaatatgttattaattattgttgtatataagattataaattttcaaaataatatgtatgaaattgaatataaatattcaaatgatgacccattactcagtagaaagttttcttaaatctatttttgactgttgtaatattttttttatgtattgaacagtttatatttgtttttaaaaattcaaataatggcatatgcgaaaaaaaactctaattatttttttataacgatgatattattttttgcaaaattagaatcatataaagatgagaggtgaactataataattaataaaaaattaatatgataatttagatatcaaatctaatttgtttattttaattggttacttttttggaaaataataatgtattttgtttttctaattaaattaaattaattaaaatttagatatcaaatcttatatgttgattttgattggttacttttttggaagctaatatatatatttgttgtttttggataatcatgaattcagttttatctagattttgttttcaaaaatattaactaacctgaatattttttggtactatataagtaaaattgcgtggtaggaaattttgacctaaatctgtgttgatcaattttaggttctttctcaaaacgcattttacaagtagggaatagaatttgaagaagcattgtgtttaggttaaaaactgaatctctctattcatcttccttttttattttcatataaatatatatggtttagaaatcaggtgatgttcacttgattagattcatcttctttttatacattgaggtttgtgttttcatcttctcactttctctaacatatctgagtgtacaacgcaatccacgattttgatggctattctcattactatattatttagaaaccttaatctctttactgtttagattcacataaaaattcgtacgttgtgatttttttttttttcagttcgtgtatagacctaggcatacgattttcccttttgttctttttttttcatgtttcggattttcgtgtttaagaaaaagaatctgtaaatatgtttgtttgattcagtttaaaaggaaaaaattacatcatgtatttcgatatatcctatatttaaatgtattttgtatatattctagaaattatcaaaaaggtataatttcatcttctttctttctttcacagattatgatgtttcatcaaatggacaaaatatcgacaccatgcgcggcatgcaagcacctgaggagaaaatgcacagaagattgtgtgtttgcgccttacttcccctcgactaaactggacaactacgaagcggttcacaaggtatttggagcaagccatgtagctacacttatcaacagtcttcacccatgccaaagagaatttgccatggacacgctcgcttgggaggcacaagttcaagccaatgatccggtcaatggctgtctgggtatcatttacaacctccttagtcagattaaagacttggaagaacaactcgcgatcgtcaagaacgaacttgcctcttactgtatcgtccccacatttgtgccaccaccttcgatgacgaatctggaaatgcataacaatcctatgatgataccggaacacacacctaataacggtggttgtttaacgggtcaacagttgtataacgaggctcaacgttttgcatccaccacacaaagcgctcaaatgcaagagacgcagacgcagcacaatgagagttaccgtgacaaaagttcttatcaaaaatttggaccatgctttaacctacattaatcattatcatattgaatcttgataacaatggtaagcttttaaaatcttttaagtcttgtctagtcgtttgtataactacattaagtgcccaatgccttttaatttattctaaactcttttattatcacttttgtttcgtttacaattaaaacttctcttttatgtgttttgaatttctcagcataatggtatgaactgtcataattctttatactgtatgatggttctcttttctttggtgtgggaaatatatgtctttttacgtgattgaatatataaatactattattgtttttagttaacaaaacatccgttatcgtccagcggttaggatatctggctttcacccaggagacccgggttcgattcccggtaacggagctttttttggtttttaagattcgaaaaagaactttttgcctcatcactatctttacgttaataaactcagcggtagtccacgccgtaaaccttaacatttctaatccggatctttagtcaatttgtttatttcttttacaaacactcccaacattcaataaggatcttgtgtagaaactacactctacacaagtgcacaacgccataaggcccataacaaaaaataaaggtaaaataaaggtgcacttgtagttatgtacaaacgactagacaagagtcataaaattagaaaagtttaatgtaacattgttatcaaaactattgtttgtatggctgcaagaaaaataaacatcttatgatctataagaagcttttttttgttaatgacaaaaacaatttttgacttggctcaaaactacgatcacaaatttacaatcgtaacaacccgtcttgtgagactcagttggtgttgcaacacatcaaccctaacctctttttttgtgaattcttctgtcataccatgtaggttattggttcgcttgacgttcctcaaacccaagacctcaccctttaacaatctttccacagaacaagctgttaccaattaatctgccaaaaagacccacataaaagaagttagggttatgcgtagcgtctgctggatccgacgaggcggattgttacaacaatcacccacatcaaattgggatattttagaagtaaacaagtacaagttaaggtaaaacaatacatatacatgagaattcaattaatttaaaaaagacataacaatttacccacctcacaattcgtactcattcaattacaaattttaggagcaaccaaattgtgttgataatatagaacagtaaaattaatattgataccaaaaaaaaaaagatgcagttgtatcatcaaaacgcggcggcctttgtcgtatcaaatcagataactaaaacaaataaaacggtgtcgttgcaaacatctctctcctcttatctaacttagagagcgaccaataattgcctttgtctttatcattcgtcgttgatccatctttttcccccaaattcatatccttccttagatatttttctccttcttcttcttcttctagattccagctactccagaagattcttcgacttaatctgatgtgattaggaagagtaatagagcaagagaagaatcagaaaaaatggatccggcgactaattcaccgattatgccgattgatttaccgattatgcacgacagtgatcgttacgacttcgttaaagatattggctctggtaatttcggcgttgctcgtctcatgaccgatagagtcaccaaggagcttgttgctgttaaatacatcgagagaggagaaaaggtttcggttttttttttggaatgaaaaaaaaagatttgatatttataggctttagtgattgattgatgatcgggttttttagtttccagatttagtagctttcctttttagagcttgttgatccagtcgtctttaatggtggttacttactgttactatgattattaatgctttttgaaaagttttgatatttctcagtaaatgatgataccttttttttcgtgtgttttgcagattgatgaaaatgttcagagggagattatcaatcatagatcattgagacatcctaatattgtcaggtttaaagaggtatatataaatactacaccttgatgtttcttctgtgtgtttttgtgttataagtcagtgtggtttagagtttgtttttgtattggtttaggtgattttgacgccttcccatttggctattgttatggaatatgctgctggtggagaactttatgagcggatttgtaatgccggacggtttagtgaagatgaggtctggattctgattcatcttactgtgtttcagtattgatgtgtgattactgatcttttgtgtttttttatgtataggctcggttcttctttcagcagcttatatctggagttagctattgtcatgcaatggtatgcagagctgtgttcttgcaacttacttgaacttttctcgttctcgtgtttattactctggatttaattctgtgtttgattttcctgtgaagcaaatatgccatcgggatctgaagctggaaaatacattgttggacggaagtccggcacctcgtttaaaaatatgtgattttggatattccaaggtactatttctctcaaggtttgttttctgaattctgagtttatatatgctactgtatggccaagcgcagaacagcgggaacatcttgtgattcctcattgtgcttcttatcgttcatgctctttacctacatgtgtttcctactaagtgtctgtttttgctttgatttctgctcaagtattaactatgcgtttcttttcttcagtcttctgttcttcattcccaaccaaagtcaactgttggtactcctgcatacattgcaccagagattcttcttcgacaggaatatgatggcaaggtaaggccatgagaccaattcctctgtctagagttaaaaccagcttatccatatgactgtttataccataaatattctttgagttgtcctctgaaatagctggtgttacatttttccagcttgcagatgtatggtcttgcggtgtaacattatatgtaatgttggttggagcttatccattcgaggatccacaagagccacgagattatcgaaagacaatacaagtaatgttttttaattgttcttgatgtctcattcatcatcatccacaaccttgttatactcattatccttcaaaagttgattgtttatgtttgcttctccattgattttaaaacgcagagaatccttagtgtcacatactcgatcccagaggacttacacctttcaccagaatgtcgccatctgatatcgaggattttcgtggccgatcctgcaacagtaagttttacacttgaaacaaactttgttcgttaaggcattttgagttctaaatagacaaactgagagacctcaatgtggcagagaataactattccggagatcacatccgataaatggttcttgaagaatcttccaggtgatttgatggatgagaacagaatgggaagtcagtttcaagagcctgagcagccaatgcagagccttgacacgattatgcagataatatcggaggctacgattccgactgttcgtaatcgttgcctcgatgatttcatggcggataatcttgatctagacgatgacatggatgactttgattccgaatctgagattgatgttgacagtagtggagagatagtttatgctctctgagattcctgaggacaaagtctgttttgtccgtactgttgagacacaccagtggagttttgtcttagctccacacactccaccgttcatttttggatcgtttgttgttttttactctacaagctttggattcacatacatatgtattgtaatgtaatatgtaatatattttatgtatttttctttgttttaataactattggcacattttatacaaatgatatggtactagatctgaaaaaaaaaaaggaattctccaaattataacaaagttgaaatctacattttacattattcatatgagtgatttgacacaattggtacatcaagaatcatcacttcacgacgtatcggatcgcatcgcacgcctttgttatgttttttggaactccagccgatgtggatatcaatcttcggtttcggtctgtttcttcgacaatatcttgattagtactctgcgatgtgctacaattgctgtttttgttaccgtaacttcctacgaaagagtgtgaataagagatgtatgggccgcttgataacgaagtagtatggaatcagaagcatgtacgcggtaacgaaccgttgaaaagtgtgaaagggatttttaaagaaaattagcaaacggtatgtgttggaaacatacatatattaataaataataaaatattatatcatatttaaaatataattaatctcacaaacaatacatctacatatatagtcattttaacaaataaatcctggatttggaacttatttactactcaatgccattaacattaaatcttttctccaaaatgattaattttactataaatttcttgatttaaattgtcaaccacattatcaatcaaatttaatctaaacaaacttcaaatcaatcccttaaaattagcataaacatatttcttaataatattttaaaacaaagttttttgttaaaacaaatattgttcttactaaacgttttttgtttaaataaataaatcatgtatttgaacttatttacaatatcatgatactgacattaattatttaggaatgaaaatatttaattaaagatttaaatcttctaaatccttacttttagagttattatgtcattacctaaaaggctcaaaatataaaaattttaaatattattatttatatatttaaactttataaaacgttattaacatttaaacttataaaatttttaatatatcataattttttaacatcattatataatatatagagtttaagaaaatcttaatctttccttcatattttgtgactcgaaattttaaaaatgaacatatattaaccaattggcgaaaaatgtgtgcgttcaacgtccatatcaactaaaatatttvgagcatgattcataaacatatcataaataagattaacattaataaaataatggtttttttacgggacgggtttggcgggacgggtttgacvggacgttacttaataacaattgaaaactataaaataaaaatattttataaatcgatacaatttacaaaattttacatatactaactttaaaatataaattgtcttcgcgatgtaccgcgggttaaaatctagtgatcattaataattcaactaaaagtaacaaaattaaatagtcttttactttttttatagataattaaatcaaatagtttaaagtcaattttagatcattgtcagtaaaaaaacatcattaaactcaagtctttcaaagttaatttaattcaatttatgcaaaaaaaaatcatgaaacatagatctcaaaagaagcgaaataaaaagattattgttaattattattttgataaaattacacatagattgagaaagagtttttcaataattatggggaataagagagagagagagagaaatagatttccgaaattgattacaagaagaaataatttcaacaaagtctctgtttttttttatcaagctcttattttactacaagcagaaataacttcagcaagtttagtgtccatttcagatatcttggccttgatttctttgatatcgtcccattgatcaacttgaaaatccacaactattatgcttcttaatgcaagttctatcgtttcgattgctttgatgtacctaaagataaacagaacaaacataatactcgtgttatttttccacaacatgataggttttgtacgtttagtgtttggagattatcgaaatcatgtaaaaaaaattgttacaaagaagaagatatttttctctaaaccattaaactaagaaattaggcgatccaaaaaccaatagaaattcatgtcatatatacgaacctgtacttcctgagtacttgtctgcgtctgagtttcggataagcctcaacaaaaacataagctaattcaaggaaacacgtgagacgttcgttgacttcccttaatggtgaaccttccactttactccgcttatcgatttgaaccaaaaacggctcgatactaaggattgtagcgcagagacggtgtacgaaagatatatctcttgcttttctgaaccggtcgtaaatggcttgggctccaactccaagaacagcccctatcgcaagctcaccaatcggcattttttgaaagtagttgtttagctctcgaggtgaatatagaggaatctatgtacatggaaggatggaaccatattaaatagttttatgtttaacaagttaacgagtggttttaattatatgaagacaattcaagagattgactcatagacttagtactgtacgggtcaacaactctctctttttctaggtaagaggagatcgttggatctatatgcaagttgtcgtgagtattaaattacgtagaatattattgaattacgtcgaagaagcgagagtcaatctcactctcaatggttaacttgtacatttagaagaaggaaaaatcaacgaagttggctgagtaagaagtgaagaagaaaaacagtgaagaaagccaaaaagcagaagaggaaaatggtggtatcaactaaaaatatttcaacaaaggaagttactactaaaaatatttcaacaaaagaagttactactaaaaataaatactttgcatgttgcagtatatatttaaaatttagaaataattatatctattaaaaaatcattttgtaacagatgttcgattatgatatatagaattattttgtagacgttttataaaatagtttaaaaaattatattgaagatatgagatgaaccacaatacgtatttttatttttcgtattttcaaataaactcttattattatatgaaatctgaattagcccagaatattattagatttggtttataatttaatctcaaaattttcttccaaactgaaaacagaaaaaaaaaaaaaaaaaaaaagaagaagaagaagaagaagttaaaaaccactaatctgaaagatccactctaatttgtataaatttttcgttttaagttcaaagatgggatcaaatcaaatgagaagaatccttaaaaactttcatctttatgtaagaagcaaaagcaaatttagttaagcttttttctaagttctttatatcttctttcagcattaattcattatccacaactttgttatactcattatccttcaaacttgattgtattgagtttgcttctccgttgatcctaatacgctaagttcaactctttgtaacaactttgttctttaaagcattttgagttctaaataaacaaattgagagaccaatgtggcagataatcgtcattttgagatcgtttgttgttttttactctacaaactttggattcacatacatatatatatatatatatatagatatatatatatatatatattgtaatgtaatgtatagtatatttctgaatttctctttgtttaataaccattggcacatttatttattttcaaagtatgtcattagattattcatattaatacatatatatgagtcgtttgacacaattgggacatcaagaatcatcactgcagaacgtaaatcggatcgcacggtttgtttttcctgaacaccagccgatgtggatttcaaacttcggttgaggcattattttgtcaagtttagtgctgatttcagacatcttggccttgagttctttgatatcagccaattgattaacttgaacatccacatctaccacccatcgtaatttagcttcgaactctttgatatctctcatgtacctaaagataaacaacacaaatataatacacatgttattgacttaattcatagtaaatgttaggttttgatagatttagtactgttgggagtttatggaaatcacatataggaactatttagcacaaacctgaacttcttgcgtacgtttctgcgtctcagctccgcattctcctcaacaagagaaacagcgttctcaaggagaagcttaagacgtttattgactttcctcgatgttgaatcttccatttcttcactgaacttatcaatttgaaccaccaacggtgtgatactatcgattgtagcttcgagacggtgtaagatgaatcttgtggttacagatctatcttttgctcttttgacggcctcgtggaggacactgagagcaagtccaagagcaccccctgcggcaacctcagcaatcattttcttgaaattagtttgttagctctcgaggtgaagagtttttgatgagttatattgatgatattattttgtttggtaagaaaaatataagaccatctattatattatatagaggtgaatatttataattcctttttcttctcaaatatttggtaaagtgttgctctattaattcacataatgttagtattatacacaaatattataagggtgaatgcaatgagaaatctatgaacatggaagtcttttgcttaacaattaagccgtgtagtttgtataaagtcaaacggatgttctttgtttccgtaacttcctacgaaagagtgtgaataagagatgtgtggaccgcttggtaaagtaccatgcagttagaagcatgtacggggtagtgaaacgtcgatttttattataaaataaaataataaacgatatgtgttggaggcgtatatatattaataaatagttaaataacaaaattaaatcgtcttttactttttttatagctaataaaatcaaatagtttaaagtcaattttagatcattgtcagtaaaaacatcattaaactcaagtctttcaaagttaatttaattaaatttatgcagaaaattcataaaacatagatctcaaaagaagcaaaataaaaagattattgttaattattattttgataaaattacacatagattgagaaagagtttttcaatcattattgggaagaagggaggaagaaaagaaaaaacagatttctgaaattgattataagaagaaataatttcaacagtctctgtttttttaaatcaagttcttattttattacaaagtgaaataatttcagatatcttggccttgatttctttggtatctttctaaaaaacaaatttagagaccaatgtggcagagaatcgtcattttgggatcgtttgttgttttttactctacaaactttggattcacatacatattatatgtattgtaatgtaatgagtaatatatttctgaatgtctctttgtttacgttacattggcacatttatgaagacaaaagacgtttttgattaattatattgatgatatatataaagacaaaagacgtttcacaaaatattaaaaccttaggaaagacaccccatttatcatcaatggaggtgctcttagataacaatctagaatccttatcgctttagacagctgtgttattgactagtcatcatctaaagaggataaggattggaaacgatttgaattggagaccaagtgcttggagagtaagcttagggttgtctttgtatgtgtgtatatatactcctcaagatcgatcaataacatcaagcactttttcaaccattcttagtctttacaattaatgtacgaagaggattattatttattaaattacgaaaaagaagtgaaaatcgatctaaatgattgactttttacgtagaatcgtcgaattgcatgtacatttccaccgaaattccaaaaatctgaattacaaataagttggaaccgatcgatcttgttttgtatatttacgtacaaggcagacgtacatacatgtagtttggattatcatatgtatgatcaacgcaattttcgtgaatagaaacgtgaatactaacaatttcggtgaatacctaccgtaaatactaacattaaaatctatgacttcttaaaataataatcaatcaaacttttacatttgattttatattttcctcagtttttaggcctatgatacacctgccttctcaaaatattagttccgtgatgtttgctccatctaaggtggatatcgatcttcggagcatccagctctcgcttccaatgatggatcaacgctttcaactatgttctgattagtattatcctcatcagatctaatacagtatattggttagtattcatttcaaacatcttgcccatgagttaacgttcattaatatctgcccacttttgtatgttattacatgaaaacaaaaatacgataggttctgaattacgctgctgctaaaatacacatgcagcttgtaaatgtgcggcgtaaccttatatgtaatgttggttggagcatatccatttgaagatccacaaggccaatagattatcgaaagacaatacaagtaaagcttcttttctaagttctttctatctcattcattatcctaaatcgcagagaatccttagtaccatatactaaatcccagagtacttacatctttcaccagaatgtcgacatctaatatcgagggttttcgtggccgatcctgctacaataagttcattttatatacaaatgatagcattacacctgaggaaaataaggaattctcctaattatttcaaagttgagtctacattttaaattaagtcctactatcacatttgagtgatttgacacaattggtacatcaagaatcatcacttcaagacaaatcggatctcacggtctttgttccgttttcttgaactccaatgaatgtggatatcgatcttcggtttagacccatcactcgaacattcaacaatttcttctaaagatcggtctgtttcttcgacaatattttgactactactttgtgatgtgctatgattgctcttgaaacaaatacaatcagttggttgacgagtgatttcatcaagtttagtgttcatttcggacatcttggccataagttctttgatatctacccattgattgacttggacatccacatctaccatccatcttaatgaagcttctaactctttgatccttctcttatacctaaagatcaaaataacacaaaaatattaatactcatgttatttcattaaaatgataagttttgttagatttatattgagaaattacgaaaatcacttatagaaatgctcatctaaaaaaagatttcacttgaaaaaatcttgaatcgaacttgaagtaaaccgttcctagctaatccaagatatcttattttaaaacaccatgaaattaatcactgtaagtcagtatctcaatctgagattctaatggtatccatcttttaagtagtgacaaaacaaacaattttttattttatttttaactattaaatttaagaaataagaagacttcgtatgaaccaaaagaattcatgcatttcatggattgtatacaaacctgaacttcttgagtaagtttctgcgtctgagttccgcataagcctcaacaagagaaacagccttttcaaggagatgcttaagatcttcgatgactttcctcggtgaatcttccacttcttcgctaagcttatcgacttgagtcaccaacggagtgatcctaaagattgtagcatcaagacggtccaagatgcatcttgtagttagagatctatcttttgcttttttgatggcatcgtgaaggacttggagagcaagtccaagagctgcccctgccataatctcactaaccggcatttttctttgggtcaagagattagctctttagctctcaacgtgattgtatggagcaatctatatacatgaaaccattgaagaatcttttgtttaacaaaacgttttgtttttgatgaactatatgaagacaagagacgttgacttaaagacttagaattagtcgactctctttctttctaggtgaaaggagataattggatctatatgcaagttgttgtgatttttaaattacgaagaagggagaatcaatctcaatgattgactttaacagtaggattaaatgttgacttgtacatttgcattaaaaaaatccaaaaatcttaatttacatattagttggaaccaatcttcttttttgtttatacaagcaggcgttatatgtatgataagttgataacacaatattcgtaagtataatttttttttttttttacatttgattattttctttctcaattttgctggtctatgacacataccacatcaaaaaaaaaaactaattcaaaacgaagcgaattccgtgatctttgctttgcttgctccatcgaaggtggatatcgatcttcggcttagaatcgctatttgagcatccagcttttgcttccaatgatggatcaactctttcgactatgtccatattagtattatcttcagatataatacaatctattggttgacccatgattttttcaagtttagtgttcatttcagacatcttggccataagatctttgatatctgcccattgattgactttaacgtcaacatctaccatccatttcaatgagccttctaactctttgattcttctcttgtacctaaaacaacagtacactttagtatgtaattaaatacatacatgaaacaaaaaaatgataggtttttgtttaattggtgtctatataatgttgggagatcaagtaatatgtagaagtattcatgtatctaagattaaaaaaaaaaatcattatttcatgtatatatatacgagaccaaaagattcacaaacctatatttttcaagtaagtttctgcgtttgagctccgcataagcttcgacaagaaccacggctttctcgagaagatgtttaagatcttcgaagactttcctcagagattcatcggattctttgttgagcttttcgaccttagccatcagtggagtgatcctcaagattgtagaatcgagacggtccaagatacaacttgtggttaaggatctatcttttgctctttggatggcctcgtgaaggatttggagagcaagtccaagagcagcccctgcgataatctcggtaagaggcattttgtatagttttttttaaaggattacttaaattacaaagagatttattgcaattagctttagctctcgatcgcacgaggttagtatatgaatgaatcaatattgaattggtcttttgacaaaaacacttatttggattgattttttccaacttgttgtgattatggaattaaagaaaaaggagtagagaaatagtctcaatgtggattaattttttctgttaaatattaatattttgatcaacttgttgtgactatgaaattaaagaagagaagagcgagcagtgaagcattctcaatgaaaattaatttgtttgtttcaagttatatatgaaagtaaagaaaaaaagggaagataatcatattcgaaatgatcgaatttgtacatacgttggattaatcactgacgtgtactgaaatctcctaacgtttctgtccattctcgtttttctaatttatattggtagaaatattgtatattctatattgacattatcggtgaaatattatgatgactatcatatggaaactcttttagctaagtttagattaaatatagaaaaagtttttctttcatattgatcctactaattatagtgaaatgtttcgcttacctctttgttaagtttggtttacagctagtagttgttaaagcaagtgaaaattttatgtttccgcatttgattgtgcgtttgtgacccataagaaggatttacaagaagtttggttagataaaaatcttttataataagagaaagttttttctaactttgcctaccaatgcgatatttgacactttactttgatgacacgtgtacttatcattaattataatttaactaaaataatttattaatagatatatcttatatttagaaaaatagtcattaaactatgaaattactacatattaaactacaaattttgaaatgaatttttttctaccataactgcaactcccttaactaattacaaaatcaaatctttaaaattttgacatgatattatgttttagagtacaaagtaatcactatttatatgtttttgataataaaaaattattatatttactattatagattaccattaatgcatactcttaacattttcttacctgcttaaccgtaataaggtattaaacactatttaggagtactttgatccaaaaatttatgaccctttatatctcataagttacaatattttgatcaccacattaaagtacaatttatgacttaatatataactaaaaatggcttaagaatgcaactatataacaagcatggcttgagaaaaagcaattggcaacctcaagtgtctcaaatgctcagctccaaatacaatatccgtttttcatttttttttgcatattttagttgtacatcgatatatctttctagagtttggatgtgtttcttacaagttcagtaaacatctaagctctacactaacttatatttggatatagtatattctttgaaaattcaaattctcaatttcacaacaattatgggatatgctagctaaataaatatttggtaaatacatgcaaagttttctctggtgttgttaaagtccattgagatgcataattattcttgctagaataatcaaatccgaaagtacctactcattatccgtgattgtgtatctccatttgttttttctgtctaggcaaggaagacaaagaagaaggttgaagaagttcatgaaatagagggaaattcttagactatgaatactaaagattaccatatagaaaagagtatttacttgtttttttggtctacttcctttatttatttaagtttgacaatgttaattgaaaccttattttcatgaaatagagggaaattcttagactatgattactaaagattactatatagaaaaaagtatttatttgtcatcttagtttacttcctttttttttttttaagtttgacaatgttaattgaaaccttatttagatattcactaaacaaaattaagttgtttcctcacagtaccaagacaaccaaggggttccgaaccaactataaaacgattaatttatttaaaatgtagttaaacattttaatgtttatttagtcataaatattatatatatatatatatatatatatatatatatatatatatattattaaattattttataacattttttaactttgcctaccaatgcgacatttggtactctactttaatgacacgtgtacttatcattaattgtaatttgactaaaataattttttaatagatatatcatatatttagaaaaatagtcattaaactatgaaattactacatattaaactacaattttgaaatgaattttttctaccataattgcaactcccttaactaattacaaaatcaaatctttaaaattttgacatgatattatgttttatactacaaagtaatagttcactactgtaaataattctccaattttacaagaatttttccatctttagttttttttataccataattttataaattttaccatgttctaattttaaatcagaaaatcctctaaaagtttaactattagttacatttctttgttctccttattttaacttctcgaatcatattaacatagtttttgtaacatcagtttcttctctttagctttttttacacaagaagttttttgtcttgtgtcaaaagctgatcatgaattggagagaagtaataatgattccttgatgagttagcgtaatgccaacagtcgttgccaatagacagcattatccacagaaaacatgtattttacataatttagggtagaaaatatatatacaaacttttcactcatttgctataggtagttaatttattttgtcctaccacatacaaatctacattacgctaccaaaatatgctaaatctttttatccatataagaagtttaatttttaataattagatatcttaatcaaatttctagcgtcatgttcaaaaccgatgatcataagctatgtcttccatcacaatgtcgaatgacactatacatggaatcatgtgtcgtgtgaaatcgtagatcataacattcttaaccaagactcaagttaatctctatctcagaaccttgtgggttgggcgtaaaacatctcggggttggctcatctcattgggcaaagagaaccccaatttatttttaggacttttcttttcccgttcccccacctcagtatagtgcttcgcttccggttcttcggaagaatcaacttacctctatctttttattatataaaattgtgttttaccgatataacaacataagatatatatatacatgaatatagtggaaaatgagagagtgattatttataagatggaaatgaaaaattgataaacgtaaataaacgtaaatattacaaataatttgaataaaagggttttagtcaagtaaaaaggttttccaagagacatcttaaatcatatttttaattactttcgagttatatattaataattatagataaattatatatataatttgtccttttttgtacatttctcatattttatatgactttttttttcctctgaactgacttttttcctacgaagaaggttcatctatttttttttccaagacaattggtaatacatgcatttgcattttcacaaaaacaaatacttaatatttaagtttttttaatagtaaaacatactgttttcacaaaacaaaaaaagcaattgtgtgcataatttttaatcaactaatgtgtctttctaaacacatcatactttgtaaacatcaaaattactattattaattattaattattttgagcgatatattaacaattataaataaattttatatctaatttaatataaatattattctatattcaaaattaaaaccgaatatataattcacccatcgcgtggacaacttctagtaatataatataatagagattcagatagataatagttggttcttaaccaaatttcaactataatagagtacttatgatgcatcatgcacatttgtatctcatacgaataagaaactaagtgtgcaaattgatatgtatccatgcagagttagtttggattttggatgatttttatctcatagtcatagtcaattgtctcgcaatcaatttatgaagacatatcaaatgttgtcaaatcatttgtgtatgaacatacgttacgtgatgttcaatctaataagagatattgtatataaaaggcacaagagttatttgtcttgtcaaccattggaatgttctcagaagtttataataacataaggttgtgaactaattaatatattagttataccatttccagtaataacaatataaaatgcttaacaaaagtaataagataaaatgttggtaaattaatacttacatcacacattcacctacaataattccaacactccaaaaactatatcacatttgttgagttttttttttctcaaaaaacattttgtggtctaggacacgatcgttacattaacaaatcatttgaagatgaaccggatctcgtgctctttggtctggtttttccatcgaaagtgtatatcaatcgttggctttgtgcatccatcttcttcagaagatcgaccagttgcttcgatgatatccattggttgacgcgtaatatcatcgagtttagtgttcatttcagacatcttacccataagttttttgatatccagccattgattgacttgaacatccacatctatcatccatctcaaagaagagtccaactccttgattcttctcttgtacctaaagaaagattcacaacaacaacaaaaaaattgcatgaaaaataaaagattttggtaggatatttttttgttaaagtatatactttggttggtataatttaacactacattacatttacatatggaaatgattaacgacattcacaaacctatatttcctgagtaagtttcggagtttaagctccgcataagcatcaacgagacgaatggctttctcgagaagacgtttaagctcttcgatgacttttctgaaaggttcatcgacttcttcggtgagggtatcgatcttaatgacaaatggagttatcttatggagtgtagcatcgagacggtccaatatacatcttgtgattaaggttttctccttggctcttatgatggcctcgtgaaggagttggagggacaatccaagagcagcacttgtaagaagctcaacaagaggcattttcttcagaaaagctttaaagctttgattaaatggagactaatcgatattatagaaggaatttaaggacgtggttggctttaacttcttgccggaggttaattatcgtatgactctatgtggaaatggtctttgttaacaaataattttatttctaagtaaatacacatacttgaattaactttttcaaattgctctaattttgtaattttagaatcttttgactttgtgtagttaagataactttcttcaagttgttgtgaactaatgttagtatatattctcatgtatcccgttgaacaaaaaaaaaatatcatgtgtccaacacacagacgcacctatagcgcgtgtaatacaccaccagaaatctccaaaattcagggaaatcgagttcttttttaagcgcgtggtcggaaattcagggaaacattccttttgggcttttaaaaactattaaatgggccttaggttgtgctgtatgcaacttaatcaaggtaggaaggatcaaatcttggatacatggtgattgtttttgtctattaagtgaaacgcaaagcaacgatcatggatcaggaacactattttgaacggtatggtcatcaaatcttacttgtgacacaggctgcaaaatcccaaattgggaaagtcccaccattctaaagtagatgtagacccttgaggtaaacatgtgctattcattgtggaaagtgatatttatcttgttctcatcttctctatcattgattgtagaagcatctgatgaggaaagaggttgtggtaggaccttgcttctacaatcttttcaagaggtcttatcccaaaatcataaattcataatgatttatgaacaagaaaatgttcatgtattgttcttccaatcaacgatggtgtcatatatgtcctctaaaatcactttccccagctagatcagtctatattaaaatgtctcacatagatagattgggtatttttactctaatatataaggcaacatgtgtcataaaatataacactgctcaatattattattttaatatatgtgtatgagaaagaattc

SEQ ID NO: 2 - protein, 148 aa, Arabidopsis thaliana
MPIGELAIGAVLGVGAQAIYDRFRKARDISFVHRLCATILSIEPFLVQIDKRSKVEGSPLREVNERLTCFLELAYVFVEAYPKLRRRQVLRKYRYIKAIETIELALRSIIVVDFQVDQWDDIKEIKAKISEMDTKLAEVISACSKIRA

SEQ ID NO: 3 - protein, 174 aa, Arabidopsis thaliana
MIAEVAAGGALGLALSVLHEAVKRAKDRSVTTRFILHRLEATIDSITPLVVQIDKFSEEMEDSTSRKVNKRLKLLLENAVSLVEENAELRRRNVRKKFRYMRDIKEFEAKLRWVVDVDVQVNQLADIKELKAKMSEISTKLDKIMPQPKFEIHIGWCSGKTNRAIRFTFCSDDS

SEQ ID NO: 4 - protein, 213 aa, Arabidopsis thaliana
MPVSEIMAGAALGLALQVLHDAIKKAKDRSLTTRCILDRLDATIFRITPLVTQVDKLSEEVEDSPRKVIEDLKHLLEKAVSLVEAYAELRRRNLLKKFRYKRRIKELEASLRWMVDVDVQVNQWVDIKELMAKMSEMNTKLDEITRQPTDCICFKSNHSTSQSSSQNIVEETDRSLEEIVECSSDGSKPKIDIHIHWSSRKRNKDREIRFVLK

SEQ ID NO: 5 - protein, 205 aa, Arabidopsis thaliana
MPLTEIIAGAALGLALQILHEAIQRAKDRSLTTSCILDRLDSTILRITPLMAKVEKLNKESDESLRKVFEDLKHLLEKAVVLVEAYAELKRRNLLEKYRYKRRIKELEGSLKWMVDVDVKVNQWADIKDLMAKMSEMNTKLEKIMGQPIDCIISEDNTNMDIVERVDPSLEAKAGCSNSDSKPKIDIHLRWSKQSKDHGIRFVLN

SEQ ID NO: 6 - protein, 189 aa, Arabidopsis thaliana
MPLVELLTSAALGLSLQLLHEAIIRAKEKTLITRCILDRLDATLHKITPFVIKIDTLTEEVDEPFRKVIEELKRLLEKAIRLVDAYAELKLRNLLRKYRYKRRIKELDSSLRWMIDVDVQVNQWLDIKKLMGKMSEMNTKLDDITRQPMDIIEATGRSSEEDGCTKPTIDIHFRWKNQTKEHEIRFIFK

SEQ ID NO: 7 - DNA, 4666 bp, Arabidopsis thaliana
catgaaacatagatctcaaaagaagcgaaataaaaagattattgttaattattattttgataaaattacacatagattgagaaagagtttttcaataattatggggaataagagagagagagagagaaatagatttccgaaattgattacaagaagaaataatttcaacaaagtctctgtttttttttatcaagctcttattttactacaagcagaaataacttcagcaagtttagtgtccatttcagatatcttggccttgatttctttgatatcgtcccattgatcaacttgaaaatccacaactattatgcttcttaatgcaagttctatcgtttcgattgctttgatgtacctaaagataaacagaacaaacataatactcgtgttatttttccacaacatgataggttttgtacgtttagtgtttggagattatcgaaatcatgtaaaaaaaattgttacaaagaagaagatatttttctctaaaccattaaactaagaaattaggcgatccaaaaaccaatagaaattcatgtcatatatacgaacctgtacttcctgagtacttgtctgcgtctgagtttcggataagcctcaacaaaaacataagctaattcaaggaaacacgtgagacgttcgttgacttcccttaatggtgaaccttccactttactccgcttatcgatttgaaccaaaaacggctcgatactaaggattgtagcgcagagacggtgtacgaaagatatatctcttgcttttctgaaccggtcgtaaatggcttgggctccaactccaagaacagcccctatcgcaagctcaccaatcggcattttttgaaagtagttgtttagctctcgaggtgaatatagaggaatctatgtacatggaaggatggaaccatattaaatagttttatgtttaacaagttaacgagtggttttaattatatgaagacaattcaagagattgactcatagacttagtactgtacgggtcaacaactctctctttttctaggtaagaggagatcgttggatctatatgcaagttgtcgtgagtattaaattacgtagaatattattgaattacgtcgaagaagcgagagtcaatctcactctcaatggttaacttgtacatttagaagaaggaaaaatcaacgaagttggctgagtaagaagtgaagaagaaaaacagtgaagaaagccaaaaagcagaagaggaaaatggtggtatcaactaaaaatatttcaacaaaggaagttactactaaaaatatttcaacaaaagaagttactactaaaaataaatactttgcatgttgcagtatatatttaaaatttagaaataattatatctattaaaaaatcattttgtaacagatgttcgattatgatatatagaattattttgtagacgttttataaaatagtttaaaaaattatattgaagatatgagatgaaccacaatacgtatttttatttttcgtattttcaaataaactcttattattatatgaaatctgaattagcccagaatattattagatttggtttataatttaatctcaaaattttcttccaaactgaaaacagaaaaaaaaaaaaaaaaaaaaagaagaagaagaagaagaagttaaaaaccactaatctgaaagatccactctaatttgtataaatttttcgttttaagttcaaagatgggatcaaatcaaatgagaagaatccttaaaaactttcatctttatgtaagaagcaaaagcaaatttagttaagcttttttctaagttctttatatcttctttcagcattaattcattatccacaactttgttatactcattatccttcaaacttgattgtattgagtttgcttctccgttgatcctaatacgctaagttcaactctttgtaacaactttgttctttaaagcattttgagttctaaataaacaaattgagagaccaatgtggcagataatcgtcattttgagatcgtttgttgttttttactctacaaactttggattcacatacatatatatatatatatatatagatatatatatatatatatattgtaatgtaatgtatagtatatttctgaatttctctttgtttaataaccattggcacatttatttattttcaaagtatgtcattagattattcatattaatacatatatatgagtcgtttgacacaattgggacatcaagaatcatcactgcagaacgtaaatcggatcgcacggtttgtttttcctgaacaccagccgatgtggatttcaaacttcggttgaggcattattttgtcaagtttagtgctgatttcagacatcttggccttgagttctttgatatcagccaattgattaacttgaacatccacatctaccacccatcgtaatttagcttcgaactctttgatatctctcatgtacctaaagataaacaacacaaatataatacacatgttattgacttaattcatagtaaatgttaggttttgatagatttagtactgttgggagtttatggaaatcacatataggaactatttagcacaaacctgaacttcttgcgtacgtttctgcgtctcagctccgcattctcctcaacaagagaaacagcgttctcaaggagaagcttaagacgtttattgactttcctcgatgttgaatcttccatttcttcactgaacttatcaatttgaaccaccaacggtgtgatactatcgattgtagcttcgagacggtgtaagatgaatcttgtggttacagatctatcttttgctcttttgacggcctcgtggaggacactgagagcaagtccaagagcaccccctgcggcaacctcagcaatcattttcttgaaattagtttgttagctctcgaggtgaagagtttttgatgagttatattgatgatattattttgtttggtaagaaaaatataagaccatctattatattatatagaggtgaatatttataattcctttttcttctcaaatatttggtaaagtgttgctctattaattcacataatgttagtattatacacaaatattataagggtgaatgcaatgagaaatctatgaacatggaagtcttttgcttaacaattaagccgtgtagtttgtataaagtcaaacggatgttctttgtttccgtaacttcctacgaaagagtgtgaataagagatgtgtggaccgcttggtaaagtaccatgcagttagaagcatgtacggggtagtgaaacgtcgatttttattataaaataaaataataaacgatatgtgttggaggcgtatatatattaataaatagttaaataacaaaattaaatcgtcttttactttttttatagctaataaaatcaaatagtttaaagtcaattttagatcattgtcagtaaaaacatcattaaactcaagtctttcaaagttaatttaattaaatttatgcagaaaattcataaaacatagatctcaaaagaagcaaaataaaaagattattgttaattattattttgataaaattacacatagattgagaaagagtttttcaatcattattgggaagaagggaggaagaaaagaaaaaacagatttctgaaattgattataagaagaaataatttcaacagtctctgtttttttaaatcaagttcttattttattacaaagtgaaataatttcagatatcttggccttgatttctttggtatctttctaaaaaacaaatttagagaccaatgtggcagagaatcgtcattttgggatcgtttgttgttttttactctacaaactttggattcacatacatattatatgtattgtaatgtaatgagtaatatatttctgaatgtctctttgtttacgttacattggcacatttatgaagacaaaagacgtttttgattaattatattgatgatatatataaagacaaaagacgtttcacaaaatattaaaaccttaggaaagacaccccatttatcatcaatggaggtgctcttagataacaatctagaatccttatcgctttagacagctgtgttattgactagtcatcatctaaagaggataaggattggaaacgatttgaattggagaccaagtgcttggagagtaagcttagggttgtctttgtatgtgtgtatatatactcctcaagatcgatcaataacatcaagcactttttcaaccattcttagtctttacaattaatgtacgaagaggattattatttattaaattacgaaaaagaagtgaaaatcgatctaaatgattgactttttacgtagaatcgtcgaattgcatgtacatttccaccgaaattccaaaaatctgaattacaaataagttggaaccgatcgatcttgttttgtatatttacgtacaaggcagacgtacatacatgtagtttggattatcatatgtatgatcaacgcaattttcgtgaatagaaacgtgaatactaacaatttcggtgaatacctaccgtaaatactaacattaaaatctatgacttcttaaaataataatcaatcaaacttttacatttgattttatattttcctcagtttttaggcctatgatacacctgccttctcaaaatattagttccgtgatgtttgctccatctaaggtggatatcgatc

SEQ ID NO: 8 - DNA, 447 bp, Artificial Sequence
  Description of Artificial Sequence cDNA from A. thaliana
atgccgattggtgagcttgcgataggggctgttcttggagttggagcccaagccatttacgaccggttcagaaaagcaagagatatatctttcgtacaccgtctctgcgctacaatccttagtatcgagccgtttttggttcaaatcgataagcggagtaaagtggaaggttcaccattaagggaagtcaacgaacgtctcacgtgtttccttgaattagcttatgtttttgttgaggcttatccgaaactcagacgcagacaagtactcaggaagtacaggtacatcaaagcaatcgaaacgatagaacttgcattaagaagcataatagttgtggattttcaagttgatcaatgggacgatatcaaagaaatcaaggccaagatatctgaaatggacactaaacttgctgaagttatttctgcttgtagtaaaataagagcttga

SEQ ID NO: 9 - DNA, 447 bp, Artificial Sequence
  Description of Artificial Sequence cDNA from A. thaliana
atgccgattggtgagcttgcgataggggctgttcttggagttggagcccaagccatttacgaccgcttcagaaaagcaagagatatatctttcgtacaccgtctctgcgctacaatccttagtatcgagccgtttttggttcaaatcgataagcggagtaaagtggaaggttcaccattaagggaagtcaacgaacgtctcacgtgtttccttgaattagcttatgtttttgttgaggcttatccgaaactcagacgcagacaagtactcaggaagtacaggtacatcaaagcaatcgaaacgatagaacttgcattaagaagcataatagttgtggattttcaagttgatcaatgggacgatatcaaagaaatcaaggccaagatatctgaaatggacactaaacttgctgaagttatttctgcttgtagtaaaataagagcttga

SEQ ID NO: 10 - DNA, 510 bp, Artificial Sequence
  Description of Artificial Sequence cDNA from A. thaliana
atgccgattggtgagcttgcgataggggctgttcttggagttggagcccaagccatttacgaccgcttcagaaaagcaagagatatatctttcgtacaccgtctctgcgctacaatcattagtatcgagccgtttttggttcaaatcgataagcggagtaaagtggaaggttcaccattaagggaagttaacgaacgtctcacgtgtttccttgaattagcttatgttttagttgaggcttatccgaaactcagacgcagacaagtactcaggaagtacaggtgcatcaaagcaatcgaaacgatagaacttgcattaagaaggataatagttgtggattttcaagttgatcaatgggacgatatcaaagaaatcaaggccaagatatctgaaacggacactaaacttgctgatcaatgggacgatatcaaagaaatcaaggccaagatatctgaaatggacactaaacttgctgaagttatttctgcttgtagtaaaataagaacttga

SEQ ID NO: 11 - DNA, 447 bp, Artificial Sequence
  Description of Artificial Sequence cDNA from A. thaliana
atgccgattggtgagcttgcgataggggctgttcttggagttggagcccaagccatttacgaccggttcagaaaagcaagagatatatctttcgtacaccgtctctgcgctacaatccttagtatcgagccgttgttggttcaaatcgataagcggagtaaagtggaaggttcaccattaagggaagtcaacgaacgtctcacgtgtttccttgaattagcttatgttttagttgaggcttatccgaaactcagacgcagacaagtactcaggaagtacaggtacatcaaagcaatcgaaacgatagaacttgcattaagaagcataatagttgtggattttcaagttgatcaatgggacgatatcaaagaaatcaaggccaagatatctgaaatggacactaaacttgctgaagttatttctgcttgtagtaaaataagagcttga

SEQ ID NO: 12 - DNA, 447 bp, Artificial Sequence
  Description of Artificial Sequence cDNA from A. thaliana
atgccgattggtgagcttgcgataggggctgttcttggagttggagcccaagccatttacgaccgcttcagaaaagcaagagatatatctgtcgtaaaccgtctctgcgctacaatcattagtatcaggccgttgttggttcaaatcgataagcggagtaaagtggaaggttcaccattaagggaagtcaacgaacgtctcacgtgtttccttgaattagcttatgttttagttgaggcttatccgaaactcagacgcagacaagtactcaggaagtacaggtacatcaaagcaatcgaaacgatagaacttgcattaagaagcataatagttgtggattttcaagttgatcaatgggacgatatcaaagaaatcaaggccaagatatctgaaatggacactaaacttgctgaagttatttctgcttgtagtaaaataagagcttga

SEQ ID NO: 13 - DNA, 447 bp, Artificial Sequence
  Description of Artificial Sequence cDNA from A. thaliana
atgccgattggtgagcttgcgataggggctgttcttggagttggagcccaagccatttacgaccgcttcagaaaagcaagagatatatctgtcgtaaaccgtctctgcgctacaatcattagtatcaggccgttgttggttcaaatcgataagcggagtaaagtggaaggttcaccattaagggaagtcaacgaacgtctcacgtgtttccttgaattagcttatgtttttgttgaggcttatccgaaactcagacgcagacaagtactcaggaagtacaggtacatcaaagcaatcgaaacgatagaacttgcattaagaagcataatagttgtggattttcaagttgatcaatgggacgatatcaaagaaatcaaggccaagatatctgaaatggacactaaacttgctgaagttatttctgcttgtagtaaaataagaacttga

SEQ ID NO: 14 - protein, 148 aa, Arabidopsis thaliana; identical to SEQ ID NO: 2

SEQ ID NO: 15 - protein, 169 aa, Arabidopsis thaliana
MPIGELAIGAVLGVGAQAIYDRFRKARDISFVHRLCATIISIEPFLVQIDKRSKVEGSPLREVNERLTCFLELAYVLVEAYPKLRRRQVLRKYRCIKAIETIELALRRIIVVDFQVDQWDDIKEIKAKISETDTKLADQWDDIKEIKAKISEMDTKLAEVISACSKIRT

SEQ ID NO: 16 - protein, 148 aa, Arabidopsis thaliana
MPIGELAIGAVLGVGAQAIYDRFRKARDISFVHRLCATILSIEPLLVQIDKRSKVEGSPLREVNERLTCFLELAYVLVEAYPKLRRRQVLRKYRYIKAIETIELALRSIIVVDFQVDQWDDIKEIKAKISEMDTKLAEVISACSKIRA

SEQ ID NO: 17 - protein, 148 aa, Arabidopsis thaliana
MPIGELAIGAVLGVGAQAIYDRFRKARDISVVNRLCATIISIRPLLVQIDKRSKVEGSPLREVNERLTCFLELAYVLVEAYPKLRRRQVLRKYRYIKAIETIELALRSIIVVDFQVDQWDDIKEIKAKISEMDTKLAEVISACSKIRA

SEQ ID NO: 18 - protein, 148 aa, Arabidopsis thaliana
MPIGELAIGAVLGVGAQAIYDRFRKARDISVVNRLCATIISIRPLLVQIDKRSKVEGSPLREVNERLTCFLELAYVFVEAYPKLRRRQVLRKYRYIKAIETIELALRSIIVVDFQVDQWDDIKEIKAKISEMDTKLAEVISACSKIRT

SEQ ID NO: 19 - DNA, 525 bp, Artificial Sequence
  Description of Artificial Sequence cDNA from A. thaliana
atgattgctgaggttgccgcagggggtgctcttggacttgctctcagtgtcctccacgaggccgtcaaaagagcaaaagatagatctgtaaccacaagattcatcttacaccgtctcgaagctacaatcgatagtatcacaccgttggtggttcaaattgataagttcagtgaagaaatggaagattcaacatcgaggaaagtcaataaacgtcttaagcttctccttgagaacgctgtttctcttgttgaggagaatgcggagctgagacgcagaaacgtacgcaagaagttcaggtacatgagagatatcaaagagttcgaagctaaattacgatgggtggtagatgtggatgttcaagttaatcaattggctgatatcaaagaactcaaggccaagatgtctgaaatcagcactaaacttgacaaaataatgcctcaaccgaagtttgaaatccacatcggctggtgttcaggaaaaacaaaccgtgcgatccgatttacgttctgcagtgatgattcttga

SEQ ID NO: 20 - DNA, 525 bp, Artificial Sequence
  Description of Artificial Sequence cDNA from A. thaliana
atgattgctgaggttgcggcagggggtgctcttggacttgctctcagtgtccttcaagaggccgtcaaaagagcaaaagatagatctgtaaccacaagattcatcttacaccgtctcgaagctacaatcgatagtatcactccgttggtggttcaaattgataagttcagtgaagaaatggaagattcatcatcgaggaaagtcaataaacgtcttaagcttctccttgagaacgctgtttctcttgttgaggagaatgcggagctgagacgcagaaacgtacgcaagaagttcaggtacatgagagatatcaaagagttcgaagctaagatacgatgggtggtaggtgtggatgttcaagttaatcaattggctgatatcaaagaactcaaggccaagatgtctgaaatcagcactaaacttgacaaaataatgcctcaaccgaagtttgaaatccacatcggctggtgttcaggaaaaaaaaaccgtgcgatccgatttacgttctgcagtgatgattcttga

SEQ ID NO: 21 - DNA, 525 bp, Artificial Sequence
  Description of Artificial Sequence cDNA from A. thaliana
atgattgctgaggttgccgcagggggtgctcttggacttgctctcagtgtcctccacgaggccgtcaaaagagcaaaagatagatctgtaaccacaagattcatcttacaccgtctcgaagctacaatcgatagtatcacaccgttggtggttcaaattgataagttcagtgaagaaatggaagattcatcatcgaggaaagtcaataaacgtcttaagcttctccttgagaacgctgtttctcttgttgaggagaatgcggagctgagacgcagaaacgtacgcaagaagttcaggtacatgagagatatcaaagagttcgaagctaaattacgatgggtggtaggtgtggatgttcaagttaatcaattggctgatatcaaagaactcaaggccaagatgtctgaaatcagcactaaacttgacaaaataatgcctcaaccgaagtttgaaatccacatcggctggtgttcaggaaaaacaaaccgtgcgatccgatttacgttctgcagtgatgattcttga

SEQ ID NO: 22 - DNA, 525 bp, Artificial Sequence
  Description of Artificial Sequence cDNA from A. thaliana
atgattgctgaggttgcggcagggggtgctcttggacttgctctcagtgtcctccacgaggccgtcaaaagagcaaaagatagatctgtaaccacaagattcatcttacaccgtctcgaagctacaatcgatagtatcactccgttggtggttcaaattgataagttcagtgaagaaatggaagattcatcatcgaggaaagtcaataaacgtcttaagcttctccttgagaacgctgtttctcttgttgaggagaatgcggagctgagacgcagaaacgtacgcaagaagttcaggtacatgagagatatcaaagagttcgaagctaaattacgatgggtggtaggtgtggatgttcaagttaatcaattggctgatatcaaagaactcaaggccaagatgtctgaaatcagcactaaacttgacaaaataatgcctcaaccgaagtttgaaatccacatcggctggtgttcaggaaaaaaaaaccgtgcgatccgatttacgttctgcagtgatgattcttga

SEQ ID NO: 23 - DNA, 524 bp, Artificial Sequence
  Description of Artificial Sequence cDNA from A. thaliana
atgattgctgaggttgccgcagggggtgctcttggacttgctctcagtttcctccacgaggccgtcaaaagagcaaaagatagatctgtaaccacaagattcatcttacaccgtctcgaagctacaatcgatagtatcactccgttggtggttcaaattgataagttcagtgaagaaatggaagattcatcatcgaggaaagtcaataaacgtcttaagcttctccttgagaacgctgtttctcttgttgaggagaatgcggagctgagacgcagaaacgtacgcaagaagttcaggtacatgagagatatcaaagagttcgaagctaaattacgatgggtggtaggtgtggatgttcaagttaatcaattggctgatatcaaagaactcaaggccaagatgtctgaaatcagcactaaacttgacaaataatgcctcaaccgaagtttgaaatccacatcggctggtgttcaggaaaaaaaaaccgtgcgatccgatttacgttctgcagtgatgattcttga

SEQ ID NO: 24 - DNA, 525 bp, Artificial Sequence
  Description of Artificial Sequence cDNA from A. thaliana
atgattgctgaggttgccgcagggggtgctcttggacttgctctcagtgtcctccacgaggccgtcaaaagagcaaaagatagatctgtaaccacaagattcatcttacaccgtctcgaagctacaatcgatagtatcactccgttggtggttcaaattgataagttcagtgaagaaatggaagattcatcatcgaggaaagtcaatgaacgtcttaagcttctccttgagaacgctgtttctcttgttgaggagaatgcggagctgagacgcagaaacgtacgcaagaagttcaggtacatgagagatatcaaagagttcgaagctaaattacgatgggtggtaggtgtggatgttcaagttaatcaattggctgatatcaaagaactcaaggccaagatgtctgaaatcagcactaaacttgacaaaataatgcctcaaccgaagtttgaaatccacatcggctggtgttcaggaaaaacaaaccgtgcgatccgatttacgttctgcagtgatgattcttga

SEQ ID NO: 25 - protein, 174 aa, Arabidopsis thaliana; identical to SEQ ID NO: 3

SEQ ID NO: 26 - protein, 174 aa, Arabidopsis thaliana
MIAEVAAGGALGLALSVLQEAVKRAKDRSVTTRFILHRLEATIDSITPLVVQIDKFSEEMEDSSSRKVNKRLKLLLENAVSLVEENAELRRRNVRKKFRYMRDIKEFEAKIRWVVGVDVQVNQLADIKELKAKMSEISTKLDKIMPQPKFEIHIGWCSGKKNRAIRFTFCSDDS

SEQ ID NO: 27 - protein, 174 aa, Arabidopsis thaliana
MIAEVAAGGALGLALSVLHEAVKRAKDRSVTTRFILHRLEATIDSITPLVVQIDKFSEEMEDSSSRKVNKRLKLLLENAVSLVEENAELRRRNVRKKFRYMRDIKEFEAKLRWVVGVDVQVNQLADIKELKAKMSEISTKLDKIMPQPKFEIHIGWCSGKTNRAIRFTFCSDDS

SEQ ID NO: 28 - protein, 174 aa, Arabidopsis thaliana
MIAEVAAGGALGLALSVLHEAVKRAKDRSVTTRFILHRLEATIDSITPLVVQIDKFSEEMEDSSSRKVNKRLKLLLENAVSLVEENAELRRRNVRKKFRYMRDIKEFEAKLRWVVGVDVQVNQLADIKELKAKMSEISTKLDKIMPQPKFEIHIGWCSGKKNRAIRFTFCSDDS

SEQ ID NO: 29 - protein, 143 aa, Arabidopsis thaliana
MIAEVAAGGALGLALSFLHEAVKRAKDRSVTTRFILHRLEATIDSITPLVVQIDKFSEEMEDSSSRKVNKRLKLLLENAVSLVEENAELRRRNVRKKFRYMRDIKEFEAKLRWVVGVDVQVNQLADIKELKAKMSEISTKLDK

SEQ ID NO: 30 - protein, 174 aa, Arabidopsis thaliana
MIAEVAAGGALGLALSVLHEAVKRAKDRSVTTRFILHRLEATIDSITPLVVQIDKFSEEMEDSSSRKVNERLKLLLENAVSLVEENAELRRRNVRKKFRYMRDIKEFEAKLRWVVGVDVQVNQLADIKELKAKMSEISTKLDKIMPQPKFEIHIGWCSGKTNRAIRFTFCSDDS

SEQ ID NO: 31 - DNA, 756 bp, Brassica rapa
atgcctattggtgaagttattgtaggggctgctcttggaattactctgcaagtgcttcatgaagctatcataaaagcaaaagatagatcttcaaccaaaaaaagtatcttggaccgcctcgatgctacaatctccaggatcactccgttggtggttcatgtcgataagatcagcaaaagagtagaagattctgagaggaaagtcattgaagaactcaagcgtcttcttgaaaaggctgtttctcttgttgaggcttatgcagaactcagacgcagaaacctacacaagaagcataggtttgtatagtttatataatacatgaaatacttgaaaaagtctttgtgatttcttaaaatgtttttatttggtttacataatatttatgtgttgttgatatataggtgcaagagtagaatcaaagagttagaagtttcattaagatggatgatagatgtggatgttcaagtcaaccaatggctagatatcaaaaaactcgtggttaagatgtctgaaatgaacacaaaactcgacaagatcacgtgccaaccaactgatggtagttgtttcaagagcaatgatagcacatcaccagtgttttcacaaagtagtagtagtctcgaagcaacagacggatcttcagaggaagatgaagaagaaagcccaagtaatggatctgaaccaaggatcgatatccacctgcgatggagttcaagaaaaggaagaaaagatcgtgagatccgattcatggccaagtga

SEQ ID NO: 32 - DNA, 651 bp, Artificial Sequence
  Description of Artificial Sequence cDNA from B. rapa
atgcctattggtgaagttattgtaggggctgctcttggaattactctgcaagtgcttcatgaagctatcataaaagcaaaagatagatcttcaaccaaaaaaagtatcttggaccgcctcgatgctacaatctccaggatcactccgttggtggttcatgtcgataagatcagcaaaagagtagaagattctgagaggaaagtcattgaagaactcaagcgtcttcttgaaaaggctgtttctcttgttgaggcttatgcagaactcagacgcagaaacctacacaagaagcataggtgcaagagtagaatcaaagagttagaagtttcattaagatggatgatagatgtggatgttcaagtcaaccaatggctagatatcaaaaaactcgtggttaagatgtctgaaatgaacacaaaactcgacaagatcacgtgccaaccaactgatggtagttgtttcaagagcaatgatagcacatcaccagtgttttcacaaagtagtagtagtctcgaagcaacagacggatcttcagaggaagatgaagaagaaagcccaagtaatggatctgaaccaaggatcgatatccacctgcgatggagttcaagaaaaggaagaaaagatcgtgagatccgattcatggccaagtga

SEQ ID NO: 33 - protein, 216 aa, Brassica rapa
MPIGEVIVGAALGITLQVLHEAIIKAKDRSSTKKSILDRLDATISRITPLVVHVDKISKRVEDSERKVIEELKRLLEKAVSLVEAYAELRRRNLHKKHRCKSRIKELEVSLRWMIDVDVQVNQWLDIKKLVVKMSEMNTKLDKITCQPTDGSCFKSNDSTSPVFSQSSSSLEATDGSSEEDEEESPSNGSEPRIDIHLRWSSRKGRKDREIRFMAK

SEQ ID NO: 34 - DNA, 753 bp, Brassica rapa
atgcctattggtgaggttattgtaggggctgctcttggaattactctgcaagtgcttcatcaagctatcataaaagcaaaagatagatcttcaaccacaaaatgtatcttggtccgcctcgatgctacaatctccaggatcactccgttggtggttcatgtcgataagatcagcaaaagagtagaagattctgagaggaaagtcattgaagaactcaagcgtcttcttgaaaaggctgtttctcttgttgaggcttatgcagaactcagacgcagaaacctacacaagaagcattggtttgtatagtttatataatacatgaaatacttgaaaaagtctttgtgatttcttaaaatgtttttatttggtttacataatatttatgtgttgttgatatataggtacaagagtagaatcaaagagttagaagcttcattaagatggatggtagatgtggatgttcaagtcaaccaatggctagatatcaaagaactcgtggctaagatgtctgaaatgaacacaaaactcgacaagatcacgagccaaccaactgatggtagttgtttcaagagcaatgatagcatatcaccagtgttatcacaaagtagtaggatcgaagcaacagacggatcttcagaggaagatgaagaagaaagctcaagtaatggatccgaaccaaggatcgatatccacctgcgatggagttcaagaaaaggaagaaaagatcgtgagatccgattcacggccaagtga

SEQ ID NO: 35 - DNA, 648 bp, Artificial Sequence
  Description of Artificial Sequence cDNA from B. rapa
atgcctattggtgaggttattgtaggggctgctcttggaattactctgcaagtgcttcatcaagctatcataaaagcaaaagatagatcttcaaccacaaaatgtatcttggtccgcctcgatgctacaatctccaggatcactccgttggtggttcatgtcgataagatcagcaaaagagtagaagattctgagaggaaagtcattgaagaactcaagcgtcttcttgaaaaggctgtttctcttgttgaggcttatgcagaactcagacgcagaaacctacacaagaagtataggtacaagagtagaatcaaagagttagaagcttcattaagatggatggtagatgtggatgttcaagtcaaccaatggctagatatcaaagaactcgtggctaagatgtctgaaatgaacacaaaactcgacaagatcacgagccaaccaactgatggtagttgtttcaagagcaatgatagcatatcaccagtgttatcacaaagtagtaggatcgaagcaacagacggatcttcagaggaagatgaagaagaaagctcaagtaatggatccgaaccaaggatcgatatccacctgcgatggagttcaagaaaaggaagaaaagatcgtgagatccgattcacggccaagtga

SEQ ID NO: 36 - protein, 215 aa, Brassica rapa
MPIGEVIVGAALGITLQVLHQAIIKAKDRSSTTKCILVRLDATISRITPLVVHVDKISKRVEDSERKVIEELKRLLEKAVSLVEAYAELRRRNLHKKYRYKSRIKELEASLRWMVDVDVQVNQWLDIKELVAKMSEMNTKLDKITSQPTDGSCFKSNDSISPVLSQSSRIEATDGSSEEDEEESSSNGSEPRIDIHLRWSSRKGRKDREIRFTAK

SEQ ID NO: 37 - DNA, 746 bp, Brassica rapa
atgccgattggtgaggttcttgtaggggctgctcttggaattacactccaagtgcttcatgaagccatcataaaagcaaaacatagatctttaaccacaaaatgtatcttggaccgcctcgatgctacaatctccaggatcactccgttggtggttcatgtcgataagatcagcaaaggggtagaagattctcagaggaaagtcattgaagacctcaagcgtcttcttgaaaaggctgtttttcttgttgaggcttatgcagaactcagacgcagaaacctactcaagaagtttaggtatgtatagtttatatagtacatgaaatgcttgaaaagtctttgtgattcttaaaatgtttttgttttgtttatataatatatatgtgtgtgttgttgatatctaggtacaagagtagaatcaaagagttggaagcttctttaagatggatggtagaggtggatgttcaagtcaaccaatggttggatatcaaacaactcctggccaagatgtttgaaatgaacactaaactcgagaggatcacgtgcccaccaactgattgtaattgtttcaagagaaatgatagcacatcaccagtgatatcacaaagtagtaatcaaaatatactcgaagcaacagacggatcgtcagaggaagacgaagaagaaagcccaaggattgatatccaccttcgatggagttcaagaaaaggagctaaagatcgtgagatccgattcatggtcaagtga

SEQ ID NO: 38 - DNA, 639 bp, Artificial Sequence
  Description of Artificial Sequence cDNA from B. rapa
atgccgattggtgaggttcttgtaggggctgctcttggaattacactccaagtgcttcatgaagccatcataaaagcaaaacatagatctttaaccacaaaatgtatcttggaccgcctcgatgctacaatctccaggatcactccgttggtggttcatgtcgataagatcagcaaaggggtagaagattctcagaggaaagtcattgaagacctcaagcgtcttcttgaaaaggctgtttttcttgttgaggcttatgcagaactcagacgcagaaacctactcaagaagtttaggtacaagagtagaatcaaagagttggaagcttctttaagatggatggtagaggtggatgttcaagtcaaccaatggttggatatcaaacaactcctggccaagatgtttgaaatgaacactaaactcgagaggatcacgtgcccaccaactgattgtaattgtttcaagagaaatgatagcacatcaccagtgatatcacaaagtagtaatcaaaatatactcgaagcaacagacggatcgtcagaggaagacgaagaagaaagcccaaggattgatatccaccttcgatggagttcaagaaaaggagctaaagatcgtgagatccgattcatggtcaagtga

SEQ ID NO: 39 - protein, 212 aa, Brassica rapa
MPIGEVLVGAALGITLQVLHEAIIKAKHRSLTTKCILDRLDATISRITPLVVHVDKISKGVEDSQRKVIEDLKRLLEKAVFLVEAYAELRRRNLLKKFRYKSRIKELEASLRWMVEVDVQVNQWLDIKQLLAKMFEMNTKLERITCPPTDCNCFKRNDSTSPVISQSSNQNILEATDGSSEEDEEESPRIDIHLRWSSRKGAKDREIRFMVK

SEQ ID NO: 40 - protein, 17 aa, Arabidopsis thaliana
MIAEVAAGGALGLALSV

SEQ ID NO: 41 - protein, 40 aa, Arabidopsis thaliana
RLKLLLENAVSLVEENAELRRRNVRKKFRYMRDIKEFEAK

SEQ ID NO: 42 - protein, 27 aa, Arabidopsis thaliana
VDVQVNQLADIKELKAKMSEISTKLDK

SEQ ID NO: 43 - protein, 215 aa, Artificial Sequence
  Description of Artificial Sequence Consensus
MPXXEXXXGAALGLXLQXLHXAXXXAKDXSXXTXXILXRLXATIXXITPXXXXXDKXXXEXXXSXXRKVXEXLKXLLEXAXXLVEAYAELXRRNXLXKXRYXRXIKEXEXXLRWXXDVDVQVNQWXDIKXLXAKMSEMXTKLXXIXXQPXXXXXXXXXXXXXXXXXXXXXXXXXXXXXXXXXXXXXXXXXXXXXXXXXXXXXXXXXXXXXXXXXX

SEQ ID NO: 44 - DNA, 22 bp, Artificial Sequence
  Description of Artificial Sequence Primer
gacccgtacagtactaagtcta

SEQ ID NO: 45 - DNA, 24 bp, Artificial Sequence
  Description of Artificial Sequence Primer
gatttccgaaattgattacaagaa

SEQ ID NO: 46 - DNA, 24 bp, Artificial Sequence
  Description of Artificial Sequence Primer
atgccgattggtgagcttgcgata

SEQ ID NO: 47 - DNA, 24 bp, Artificial Sequence
  Description of Artificial Sequence Primer
tcaagctcttattttactacaagc

SEQ ID NO: 48 - DNA, 24 bp, Artificial Sequence
  Description of Artificial Sequence Primer
aatggacactaaacttgctgaagt

SEQ ID NO: 49 - DNA, 19 bp, Artificial Sequence
  Description of Artificial Sequence Primer
ccacaactattatgcttct

SEQ ID NO: 50 - DNA, 24 bp, Artificial Sequence
  Description of Artificial Sequence Primer
gaaccaaaaacggctcgatactaa

SEQ ID NO: 51 - DNA, 33 bp, Artificial Sequence
  Description of Artificial Sequence Primer
ccggaattcatgccgattggtgagcttgcgata

SEQ ID NO: 52 - DNA, 33 bp, Artificial Sequence
  Description of Artificial Sequence Primer
cgcggatcctcaagctcttattttactacaagc

SEQ ID NO: 53 - DNA, 24 bp, Artificial Sequence
  Description of Artificial Sequence Primer
aactcttcacctcgagagctaaca

SEQ ID NO: 54 - DNA, 24 bp, Artificial Sequence
  Description of Artificial Sequence Primer
agtcgtttgacacaattgggacat

SEQ ID NO: 55 - DNA, 21 bp, Artificial Sequence
  Description of Artificial Sequence Primer
atgattgctgaggttgccgca

SEQ ID NO: 56 - DNA, 24 bp, Artificial Sequence
  Description of Artificial Sequence Primer
tcaagaatcatcactgcagaacgt

SEQ ID NO: 57 - DNA, 24 bp, Artificial Sequence
  Description of Artificial Sequence Primer
gctaaattacgatgggtggtagat

SEQ ID NO: 58 - DNA, 24 bp, Artificial Sequence
  Description of Artificial Sequence Primer
cgatgggtggtagatgtggatgtt

SEQ ID NO: 59 - DNA, 16 bp, Artificial Sequence
  Description of Artificial Sequence Primer
ggatcgcacggtttgt

SEQ ID NO: 60 - DNA, 23 bp, Artificial Sequence
  Description of Artificial Sequence Primer
ctgaacttcttgcgtacgtttct

SEQ ID NO: 61 - DNA, 30 bp, Artificial Sequence
  Description of Artificial Sequence Primer
ccggaattcatgattgctgaggttgccgca

SEQ ID NO: 62 - DNA, 33 bp, Artificial Sequence
  Description of Artificial Sequence Primer
ccgggatcctcaagaatcatcactgcagaacgt

SEQ ID NO: 63 - protein, 11 aa, Arabidopsis thaliana
DIKEIKAKISE

SEQ ID NO: 64 - DNA, 24 bp, Artificial Sequence
  Description of Artificial Sequence Primer
atccgcctctttcttttggttttc

SEQ ID NO: 65 - DNA, 23 bp, Artificial Sequence
  Description of Artificial Sequence Primer
gtgttacttttctacagccagag

SEQ ID NO: 66 - DNA, 22 bp, Artificial Sequence
  Description of Artificial Sequence Primer
gtctgaatccgtcaagccttcg

SEQ ID NO: 67 - DNA, 23 bp, Artificial Sequence
  Description of Artificial Sequence Primer
tccatgcttctatattgaagagc

SEQ ID NO: 68 - DNA, 23 bp, Artificial Sequence
  Description of Artificial Sequence Primer
gattgtataggttggttgatgag

SEQ ID NO: 69 - DNA, 23 bp, Artificial Sequence
  Description of Artificial Sequence Primer
gcatctcattgacctcccttatc

SEQ ID NO: 70 - DNA, 23 bp, Artificial Sequence
  Description of Artificial Sequence Primer
cagcttccttcaccgtctcatgg

SEQ ID NO: 71 - DNA, 23 bp, Artificial Sequence
  Description of Artificial Sequence Primer
ccaggaaaataacggtgacgatc

SEQ ID NO: 72 - DNA, 23 bp, Artificial Sequence
  Description of Artificial Sequence Primer
gtcatcatctaaagaggataagg

SEQ ID NO: 73 - DNA, 23 bp, Artificial Sequence
  Description of Artificial Sequence Primer
ggttgaaaaagtggctttggatg

SEQ ID NO: 74 - DNA, 23 bp, Artificial Sequence
  Description of Artificial Sequence Primer
atggatccggcgactaattcacc

SEQ ID NO: 75 - DNA, 23 bp, Artificial Sequence
  Description of Artificial Sequence Primer
tgtcctcaggaatctcagagagc

(19) United States
(12) Patent Application Publication
(10) Pub. No.: US 2004/0093633 A1
(43) Pub. Date: May 13, 2004

(54) Plant resistance gene

(76) Inventors: Shun Yuan Xiao, Norwich (GB); John Gordon Turner, Norwich (GB); Mark Coleman, Norwich (GB); Simon Ellwood, Perth (AU)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/312,222

(22) PCT Filed: Jun. 19, 2001
(86) PCT No.: PCT/GB01/02693

(30) Foreign Application Priority Data
Jun. 20, 2000 (AE) 0015122.5

(51) Int. Cl. A01H 1/00; C12N 9/24; C12N 15/82; C12Q 1/68; C07H 21/04; C12N 5/04

(52) U.S. Cl. 800/279; 435/6; 435/69.1; 435/200; 435/320.1; 435/419; 536/23.2

(57) ABSTRACT

Disclosed are isolated nucleic acids consisting essentially of RPW nucleotide sequences (especially RPW8 from Arabidopsis thaliana, and related homologues and other sequences e.g. from Brassica napus; B. oleracea). These encode a novel class of resistance polypeptides having an N-terminal transmembrane domain and a coiled coil domain and which is capable of recognising and activating in a plant into which said nucleic acid is introduced a specific defense response to challenge with a powdery mildew pathogen e.g. E. cichoracearum. Also provided are related products e.g. primers, polypeptides, transgenic plants having enhanced resistance, plus also processes for producing these, and methods of use.

The present invention relates to methods and materials, particularly nucleic acids, for manipulating the resistance of plants to powdery mildew (Erysiphe cichoracearum). It further relates to plants which have been modified using such methods and materials.

### PRIOR ART

Plant disease resistance (R) genes couple the recognition of specific pathogens to the induction of broad-spectrum defences that restrict the invader at the point of infection(1, 2). Many plant-pathogen interactions conform to the gene-for-gene model which predicts that disease will develop if the infected plant lacks an R gene for recognition of the pathogen, or if the pathogen lacks the corresponding (Avr) gene required for its recognition by the plant(3). The final outcome of a matched R-Avr interaction is incompatibility.

More than twenty plant R genes have been cloned and characterised. These are represented by proteins having five combinations of domains for a coiled-coil (CC)(4), leucine rich repeats (LRRs) (5), a transmembrane (TM) region, a protein kinase, a nucleotide binding site (NBS), and with similarity to the Toll/interleukin receptor (TIR) (3). With the exception of Pto, which is a protein kinase, all characterised R genes contain LRRs. The eight R genes characterised in Arabidopsis thaliana belong to the CC-NBS-LRR and TIR-NBS-LRR classes(4), and a further 200-300 homologues of these are predicted in its genome(6).

The characterisation and cloning of R genes, particularly those having novel structures, specificities or recognitions, allows the pathogen resistance traits arising from those genes to be manipulated. This is particularly important when dealing with commercially significant pests.

A. thaliana has been used as a model to study genes for resistance to powdery mildews, which cause severe losses on a wide range of crop species (7). Resistance of A. thaliana accession Ms-0 to the powdery mildew pathogen Erysiphe cichoracearum isolate UCSC1 is regulated at the RESISTANCE TO POWDERY MILDEW8 (RPW8) locus on chromosome 3 (8). However, although this specificity had been defined in the prior art, the gene or genes giving rise to the specificity had not been accurately mapped or cloned.

### DISCLOSURE OF THE INVENTION

The present invention is based on the characterisation of novel RPW resistance genes from a cosmid (designated B6) prepared from a genomic library prepared from A. thaliana accession Ms-0, and demonstrated to confer resistance to E. cichoracearum UCSC1 when transferred to the susceptible accession, Col-0 (9).

Briefly, the present inventors had sought to isolate the gene for resistance at the RPW8 locus from cosmid B6, believing that it would be either a TIR-NBS-LRR, or a CC-NBS-LRR gene. Interestingly, however, inspection of the DNA sequence in the DNA fragment B6 containing RPWB revealed neither a TIR-NBS-LRR, nor a CC-NBS-LRR homologue. The DNA sequence of B6 revealed only a potential gene for a protein kinase, SKP-2, and two potential genes which were unrelated at the nucleotide sequence level and at the predicted protein sequence level, to any of the other characterised plant disease resistance genes, or indeed to any other plant gene. These latter genes were named MSC1 and MSC2. Because a tomato resistance gene, Pto, is protein kinase it was anticipated that the SKP-2/Ms-0 homologue might be RPW8. This was tested by making subclones containing differing regions of B6, and introducing these into A. thaliana Col-0 by stable transformation. Unexpectedly, it was found that SK-2 did not confer resistance, but instead that MSC1 and MSC2 independently conferred resistance to E. cichoracearum UCSC1, and to three other powdery mildew diseases also. The existence of homologues in other plants has also been correlated with activity.

The genes MSC1 and MSC2 have therefore been designated RPW8.1 and RPW8.2, respectively.

The RPW8.1 and RPW8.2 proteins have 45.2% sequence identity, but are both relatively small and basic (pIs of greater than 9) and appear to contain both an N-terminal transmembrane (TM) domain (or possibly a cleavage signal peptide) and a coiled coil (CC) domain. The proteins have no significant similarity to the derived proteins from other isolated or characterised R-genes, or indeed any plant gene, and therefore appear to define a new class of R-gene product which is designated herein “TM-CC” class.

Thus in a first aspect of the present invention there is disclosed a nucleic acid molecule encoding a plant resistance gene of the TM-CC class. Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities.

The nucleic acid molecules may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

The resistance genes of the invention will generally be powdery mildew resistance genes, by which is meant a gene encoding a polypeptide capable of recognising and activating a defense response in a plant in response to challenge with a powdery mildew pathogen, such as any of the 15 isolates of E. cichoracearum tested herein; E. cruciferarum isolate UEA1; E. orontii isolate MGH; Oidium lycopersici isolate Oxford, or in each case an elicitor thereof.

As will be well understood by those skilled in the art, “resistance” should not be taken to require complete resistance to infection, but may in some cases be manifest as a reduced susceptibility to the pathogen in question as compared to a control plant. Preferably the resistance response is a specific response, in that (for instance) the gene will not provide resistance against other pathogens e.g. downy mildew fungus P. parasitica Noco2.

The activity of the encoded polypeptide may be tested, for instance, by challenging a plant in which the corresponding gene has been introduced.

Plants to which the invention may be most advantageously applied include any which are susceptible to powdery mildew.

Nucleic acid according to the present invention may include cDNA, RNA, genomic DNA and modified nucleic acids or nucleic acid analogs. Where a DNA sequence is specified, e.g. with reference to a figure, unless context requires otherwise the RNA equivalent, with U substituted for T where it occurs, is encompassed.

Complement sequences of those discussed herein are also encompassed. As is well understood by those skilled in the art, two nucleic acid nucleotide sequences are “complementary” when one will properly base pair with all or part of the other according to the standard rules (G pairs with C, and A pairs with T). One sequence is “the complement” of another where those sequences are of the same length, but are complementary to each other.

Preferably the gene is derived from the RPW8 locus, for instance in Arabidopsis thaliana Ms-0. However, as described below, the work done by the present inventors suggests that this locus may in fact be identical with the RPW7 locus (which controls resistance to E. cruciferarum). Genes of this type have also been found by the present inventors in other accessions and other species.

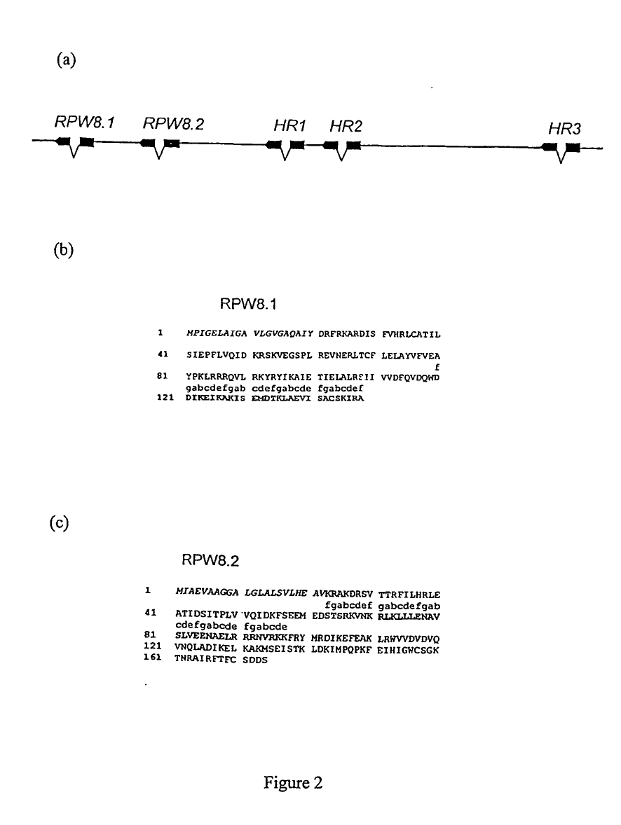

Thus in one embodiment of this aspect of the invention, there is disclosed a nucleic acid comprising an RPW8.1 or RPW8.2 sequence, which are described in Sequence Listing 2 below, which details the complementary nucleotides that define the transcription start, the first exon, the intron and the second exon, and the transcription end. Sequences which are degeneratively equivalent to the coding sequences (encode the same polypeptide) are, of course, also embraced. Thus a nucleic acid of the present invention may also be any which encodes an amino acid sequence (based on exon 1 and exon 2) of the RPW8. 1 or RPW8.2 sequences which are described in Sequence Listing 2 below. These are also listed in FIG. 2.

Further RPW8.1 or RPW8.2 sequences, from a variety of Arabidopsis accessions, are shown in the sequence lineups hereinafter.

In a further aspect of the present invention there are disclosed nucleic acids which are variants (including alleles, homologues, orthologues, mutants and derivatives) of the sequences of the first aspect.

A variant nucleic acid molecule shares homology with, or is identical to, all or part of the coding sequence discussed above. Generally, variants encode, or be used to isolate or amplify nucleic acids which encode, polypeptides which are capable of mediating a response against a pathogen, particularly powdery mildew.

Variants of the present invention can be artificial nucleic acids (i.e. containing sequences which have not originated naturally) which can be prepared by the skilled person in the light of the present disclosure. Artificial variants (derivatives) may be prepared by those skilled in the art, for instance by site directed or random mutagenesis, or by direct synthesis. Preferably the variant nucleic acid is generated either directly or indirectly (e.g. via one or amplification or replication steps) from an original nucleic acid having all or part of the sequences of the first aspect. Preferably it encodes a powdery mildew resistance gene.

Alternatively they may be novel, naturally occurring, nucleic acids, isolatable using the sequences of the present invention (e.g. those found in other A. thaliana accessions, or other plant species, as described hereinafter). Sequence variants which occur naturally may also include alleles (which will include polymorphisms or mutations at one or more bases).

Examples are shown e.g. in Sequence listing 1 which includes three RPW8 homologues HR1, HR2, HR3 from A. thaliana accession Ms-0.

Thus a variant may be or include a distinctive part or fragment (however produced) corresponding to a portion of the sequence provided. These portions may include motifs which are distinctive to RPW8 sequences, such motifs being discussed below in relation to primers. Preferred sequences are those which include the DIKE motif.

Fragments may encode or omit particular functional parts of the polypeptide, e.g. CC or TM regions. Equally the fragments may have utility in probing for, or amplifying, the sequence provided or closely related ones. Suitable lengths of fragment, and conditions, for such processes are discussed in more detail below. Also included are nucleic acids which have been extended at the 3′ or 5′ terminus with respect to those of the first aspect.

The term ‘variant’ nucleic acid as used herein encompasses all of these possibilities. When used in the context of polypeptides or proteins it indicates the encoded expression product of the variant nucleic acid.

Some of the aspects of the present invention relating to variants will now be discussed in more detail.

Homology (either similarity or identity) may be as defined and determined by the TBLASTN program, of Altschul et al. (1990) J. Mol. Biol. 215: 403-10, which is in standard use in the art, or, and this may be preferred, the standard program BestFit, which is part of the Wisconsin Package, Version 8, September 1994, (Genetics Computer Group, 575 Science Drive, Madison, Wis., USA, Wisconsin 53711). BestFit makes an optimal alignment of the best segment of similarity between two sequences. Optimal alignments are found by inserting gaps to maximize the number of matches using the local homology algorithm of Smith and Waterman.

Homology, with respect to either RPW8.1 or 8.2 or both, may be at the nucleotide sequence and/or encoded amino acid sequence level. Preferably, the nucleic acid and/or amino acid sequence shares at least about 50%, or 60%, or 70%, or 80% homology, most preferably at least about 90%, 95%, 96%, 97%, 98% or 99% homology.

Thus a variant polypeptide in accordance with the present invention may include within an amino acid sequences described herein a single amino acid or 2, 3, 4, 5, 6, 7, 8, or 9 changes, about 10, 15, 20, 30, 40 or 50 changes, or greater than about 50, 60, 70, 80 changes. In addition to one or more changes within the amino acid sequence shown, a variant polypeptide may include additional amino acids at the C-terminus and/or N-terminus.

Thus in a further aspect of the invention there is disclosed a method of producing a derivative nucleic acid comprising the step of modifying the coding sequence of a nucleic acid comprising any one the sequences discussed above.

Changes to a sequence, to produce a derivative, may be by one or more of addition, insertion, deletion or substitution of one or more nucleotides in the nucleic acid, which may lead to the addition, insertion, deletion or substitution of one or more amino acids in the encoded polypeptide. Changes may be desirable for a number of reasons, including introducing or removing the following features: restriction endonuclease sequences; codon usage; other sites which are required for post translation modification; cleavage sites in the encoded polypeptide; motifs in the encoded polypeptide (e.g. binding sites). Leader or other targeting sequences (e.g. the putative TM region) may be added or removed from the expressed protein to determine its location following expression. All of these may assist in efficiently cloning and expressing an active polypeptide in recombinant form (as described below).

Other desirable mutation may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered stability or specificity.

Other methods for generating novel specificities may include mixing or incorporating sequences from related resistance genes into the sequences disclosed herein. For example restriction enzyme fragments of related genes could be ligated together. An alternative strategy for modifying RPW sequences would employ PCR as described below (Ho et al., 1989, Gene 77, 51-59) or DNA shuffling (Crameri et al., 1998, Nature 391).

In a further aspect of the present invention there is provided a method of detecting, identifying and/or cloning (isolating) a nucleic acid of the present invention (e.g. a homologue of the sequences set out hereinafter) from a plant which method employs any of the sequences of the invention discussed above. In particular the methods will generally employ primers or probes derived from all or part of these sequences (or sequences complementary thereto) set out herein. Preferably the plant is a species other than Arabidopsis.

An oligonucleotide primer for use in amplification reactions may be about 30 or fewer nucleotides in length. Generally specific primers are upwards of 12, 13, 14, 15, 18, 21 or 24 nucleotides in length. For optimum specificity and cost effectiveness, primers of 16-24 nucleotides in length may be preferred.

An oligonucleotide or polynucleotide probe may be based on the any of the sequences disclosed herein (e.g. introns or exons, although the latter may be preferred). If required, probing can be done with entire restriction fragments of the genes which may be 100's or even 1000's of nucleotides in length.

Those skilled in the art are well versed in the design of primers for use processes such as PCR. The primers will usually be based on sequences which are peculiar or unique to the RPW sequences. Particularly preferred are the primers set out in any of the Examples shown below. Primers based on the TM or CC regions may also be preferred. Indeed, primers of the invention may be any of those which occur to the skilled person in the light of the disclosure herein, and in particular the sequence lineups shown hereinafter. For instance referring to the cDNA nucleotide sequence of RPW8.1 from Ms-0 when aligned with that of RPW8.1 homologues isolated from other A. thaliana accessions, preferred primers may be based on e.g. the first 30 nucleotides or so at the 5′ end, plus any conserved sequence near the 3′ end (e.g. between 427 and 504 using the numbering given in the lineup).

Referring to the predicted amino acid sequence of RPW8.1 from Ms-0 as aligned with RPW8.1 homologues from other A. thaliana accessions, degenerate primers may be based on any region within the first 30 amino acids or so, or (at the C-terminal) the conserved region between 153 and 168.

One particularly preferred region for use in devising degenerate primers is the DIKEIKAKISE motif at positions 142-152.

Referring to the cDNA nucleotide sequence of RPW8.2 from Ms-0, as aligned with that of RPW8.2 homologues isolated from other A. thaliana accessions, primers may be devised particularly based on fully conserved regions near the 3′ and 5′ ends.

Finally, referring to the predicted amino acid sequence of RPW8.2 from Ms-0, as aligned with RPW8.2 homologues isolated by PCR A. thaliana accessions, preferred degenerate primers may be based on appropriately conserved regions therein e.g. encoding amino acids from the following motifs: MIAEVAAGGA LGLALSV; RLKLLLENAV SLVEENAELR RRNVRKKFRY MRDIKEFEAK; VDVQ VNQLADIKEL KAKMSEISTK LDK.

When using such probes or primers, if need be, clones or fragments identified in the search can be extended. For instance if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence.

In one embodiment, nucleotide sequence information provided herein may be used in a data-base (e.g. of expressed sequence tags, or sequence tagged sites) search to find homologous sequences, such as those which may become available in due course, and expression products of which can be tested for activity as described below.

In a further embodiment, a variant in accordance with the present invention is also obtainable by means of a method which includes:

(a) providing a preparation of nucleic acid, e.g. from plant cells,

(b) providing a probe or primer as discussed above,

(c) contacting nucleic acid in said preparation with said nucleic acid molecule under conditions for hybridisation of said nucleic acid molecule to any said gene or homologue in said preparation, and identifying said gene or homologue if present by its hybridisation with said nucleic acid molecule.

Plants which may be a suitable source of RPW8 may include any of those which may be susceptible to powdery mildew. For instance, the powdery mildew fungus E. cichoracearum UCSC1 causes disease in a wide range of plant species, including members of the Cruciferae (e.g. Arabidopsis thaliana) Solanaceae (e.g. Lycopersicon esculentum (tomato), and Nicotiana spp (tobacco)) and Cucurbitaceae (e.g. squash).

Preferred plants for use in the present invention may therefore include Crucifers (such as oil seed rape, broccolis, cauliflowers, cabbages, curly kale and the like), members of Solanaceae which are affected by powdery mildew (e.g. tomato and tobacco), members of Cucurbitaceae (e.g. squash) and monocots (such as barley and wheat). Specific examples of methodologies used with some of these species are set out hereinafter). It is noted that even plants which are susceptible to certain powdery mildew isolates may be a source of sequence which is useful e.g. against other isolates, or when present as a heterologous sequence in a different genetic background (for instance in a transgenic plant).

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as is described hereinafter. Probing may optionally be done by means of so-called ‘nucleic acid chips’ (see Marshall & Hodgson (1998) Nature Biotechnology 16: 27-31, for a review).

Preliminary experiments may be performed by hybridising under low stringency conditions. For probing, preferred conditions are those which are stringent enough for there to be a simple pattern with a small number of hybridisations identified as positive which can be investigated further.

For instance, screening may initially be carried out under conditions, which comprise a temperature of about 37° C. or less, a formamide concentration of less than about 50%, and a moderate to low salt (e.g. Standard Saline Citrate (‘SSC’)=0.15 M sodium chloride; 0.15 M sodium citrate; pH 7) concentration.

Alternatively, a temperature of about 50° C. or less and a high salt (e.g. ‘SSPE’=0.180 mM sodium chloride; 9 mM disodium hydrogen phosphate; 9 mM sodium dihydrogen phosphate; 1 mM sodium EDTA; pH 7.4). Preferably the screening is carried out at about 37° C., a formamide concentration of about 20%, and a salt concentration of about 5×SSC, or a temperature of about 50° C. and a salt concentration of about 2×SSPE. These conditions will allow the identification of sequences which have a substantial degree of homology (similarity, identity) with the probe sequence, without requiring the perfect homology for the identification of a stable hybrid.

Suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

It is well known in the art to increase stringency of hybridisation gradually until only a few positive clones remain. Suitable conditions would be achieved when a large number of hybridising fragments were obtained while the background hybridisation was low. Using these conditions nucleic acid libraries, e.g. cDNA libraries representative of expressed sequences, may be searched. Those skilled in the art are well able to employ suitable conditions of the desired stringency for selective hybridisation, taking into account factors such as oligonucleotide length and base composition, temperature and so on.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below) or RN'ase cleavage. The identification of successful hybridisation is followed by isolation of the nucleic acid which has hybridised, which may involve one or more steps of PCR or amplification of a vector in a suitable host.

In a further embodiment, hybridisation of nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of are employed. Using RACE PCR, only one such primer may be needed (see “PCR protocols; A Guide to Methods and Applications”, Eds. Innis et al, Academic Press, New York, (1990)).

Thus a method involving use of PCR in obtaining nucleic acid according to the present invention may be carried out as described above, but using a pair of nucleic acid molecule primers useful in (i.e. suitable for) PCR.

The methods described above may also be used to determine the presence of one of the nucleotide sequences of the present invention within the genetic context of an individual plant, optionally a transgenic plant which may be produced as described in more detail below. This may be useful in plant breeding programmes e.g. to directly select plants containing alleles which are responsible for desirable traits in that plant species, either in parent plants or in progeny (e.g hybrids, F1, F2 etc.). Thus use of the markers defined in the Examples below, or markers which can be designed by those skilled in the art on the basis the nucleotide sequence information disclosed herein, forms one part of the present invention.

Specific examples of homologous nucleic acids are those from Brassica rapa discussed in more detail in the Examples below. The sequence of the genomic DNA, and the predicted cDNA, is shown for one each in Sequence Listings 7,8 (BrHR1), 10, 11 (BrHR2), and 13, 14 (BrHR3) respectively. These sequences are highly homologous to each other (83-97% at amino acid level) and show 44-74% amino acid identity to AtRPW8.1, AtRPW8.2 and AtHR1-3. As above, the invention also embraces any nucleic acid encoding the respective amino acid sequences (Sequence Listings 9, 12, 15) and so on.

As used hereinafter, unless the context demands otherwise, the term “RPW nucleic acids” is intended to cover any of the nucleic acids of the invention described above, including functional variants.

In one aspect of the present invention, the RPW nucleic acid described above is in the form of a recombinant and preferably replicable vector.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eucaryotic (e.g. higher plant, yeast or fungal cells).

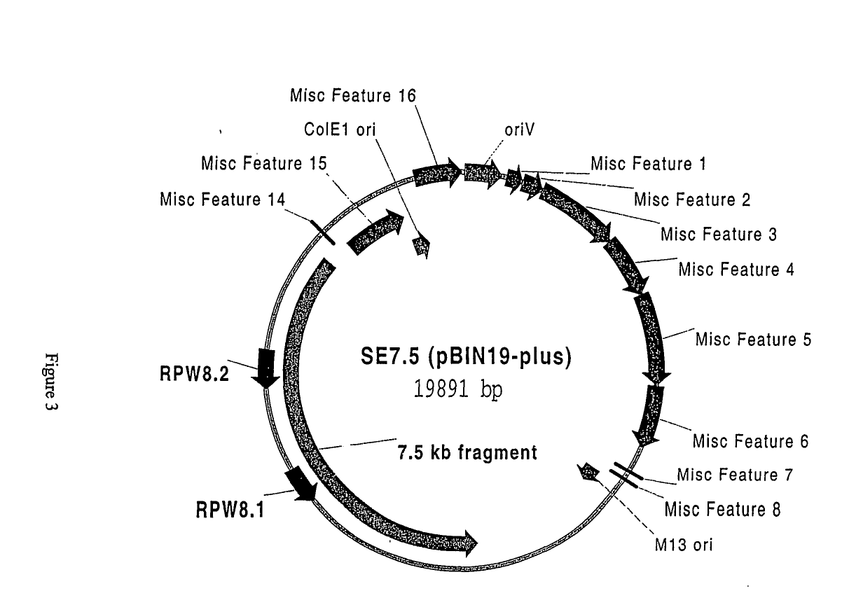

A vector including nucleic acid according to the present invention need not include a promoter or other regulatory sequence, particularly if the vector is to be used to introduce the nucleic acid into cells for recombination into the genome, such as the SE7.5 construct shown in FIG. 3.

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

Thus this aspect of the invention provides a gene construct, preferably a replicable vector, comprising a promoter operatively linked to a nucleotide sequence provided by the present invention. Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press.

Many known techniques and protocols for manipulation of nucleic acid, for example in preparation of nucleic acid constructs, mutagenesis (see above discussion in respect of variants), sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

A preferred vector is the SE7.5 construct (FIG. 3) which comprises is a 7.5 kb sequence spanning RPW8.1 and RPW8.2 in the pBIN19-Plus binary vector (F. A. VAN ENGELEN, J. W. MOULTHOFF, A. J. CONNER, J. NAP, A. PEREIRA, AND W. J. STIKEMA. 1995. “pBINPLUS: AN IMPROVED PLANT TRANSFORMATION VECTOR BASED ON pBIN19”. TRANSGENIC RESEARCH 4, 288-290.).

In one embodiment of this aspect of the present invention, there is provided a gene construct, preferably a replicable vector, comprising an inducible promoter operatively linked to a nucleotide sequence provided by the present invention.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

As shown in the Examples below, it is believed that the RPW8 promoters provided by the present invention are inter alia wound- and SA-inducible.

Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

The present invention also provides methods comprising introduction of such a construct into a host cell, particularly a plant cell.

In a further aspect of the invention, there is disclosed a host cell containing a heterologous nucleic acid or construct according to the present invention, especially a plant or a microbial cell.

The term “heterologous” is used broadly in this aspect to indicate that the RPW nucleic acid in question has been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence.

Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homolog is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

The host cell (e.g. plant cell) is preferably transformed by the construct, which is to say that the construct becomes established within the cell, altering one or more of the cell's characteristics and hence phenotype e.g. with respect to powdery mildew resistance.

Nucleic acid can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species. Recently, there has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice. It will be is apparent to the skilled person that the particular choice of a transformation system to introduce nucleic acid into plant cells is not essential to or a limitation of the invention, nor is the choice of technique for plant regeneration.

Thus a further aspect of the present invention provides a method of transforming a plant cell involving introduction of a construct as described above into a plant cell and causing or allowing recombination between the vector and the plant cell genome to introduce a nucleic acid according to the present invention into the genome.

The invention further encompasses a host cell transformed with nucleic acid or a vector according to the present invention especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewd in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Plants which include a plant cell according to the invention are also provided.

Plants in which it may be desirable to introduce RPW8 include any of those discussed herein which are susceptible to any powdery midews. The powdery mildews that affect wheat and barley are Blumeria graminis f.sp tritici and Blumeria graminis f.sp hordei, respectively, while the powdery mildew that affects tomato is Oidium lycopersici, which is also a pathogen of Arabidopsis, and is controlled by the RPW8 locus (as described elsewhere in this document). Transgenic plants containing heterologous RPW8.1 and RPW8.2 can be tested for resistance to the appropriate powdery mildew pathogen.

In addition to the regenerated plant obtainable by the above method, the present invention embraces all of the following: a clone of such a plant; selfed or hybrid progeny; descendants (e.g. F1 and F2 descendants) and any part of any of these. The invention also provides a plant propagule from such plants, that is any part which may be used in reproduction or propagation, sexual or asexual, including cuttings, and so on. In each case these embodiments will include a heterologous RPW nucleic acid according to the present invention.

The invention further provides a method of influencing or affecting the degree of resistance of a plant to a pathogen, particularly powdery mildew, more particularly to one of the isolates discussed above, the method including the step of causing or allowing expression of a heterologous nucleic acid sequence as discussed above within the cells of the plant.

The step may be preceded by the earlier step of introduction of the nucleic acid into a cell of the plant or an ancestor thereof.

The foregoing discussion has been generally concerned with uses of the nucleic acids of the present invention for production of functional RPW polypeptides in a plant, thereby increasing its pathogen resistance. However the information disclosed herein may also be used to reduce the activity or levels of such polypeptides in cells in which it is desired to do so. For instance the sequence information disclosed herein may be used for the down-regulation of expression of genes e.g. using anti-sense technology (see e.g. Bourque, (1995), Plant Science 105, 125-149); sense regulation [co-suppression] (see e.g. Zhang et al., (1992) The Plant Cell 4, 1575-1588). Further options for down regulation of gene expression include the use of ribozymes, e.g. hammerhead ribozymes, which can catalyse the site-specific cleavage of RNA, such as mRNA (see e.g. Jaeger (1997) “The new world of ribozymes” Curr Opin Struct Biol 7:324-335. Nucleic acids and associated methodologies for carrying out down-regulation (e.g. complementary sequences) form one part of the present invention.

The present invention also encompasses the expression product of any of the functional nucleic acid sequences disclosed above, plus also methods of making the expression product by expression from encoding nucleic acid therefore under suitable conditions, which may be in suitable host cells.

Following expression, the recombinant product may, if required, be isolated from the expression system. Generally however the polypeptides of the present invention will be used in vivo (in particular in planta).

Purified RPW protein produced recombinantly by expression from encoding nucleic acid therefor, may be used to raise antibodies employing techniques which are standard in the art. Antibodies and polypeptides comprising antigen-binding fragments of antibodies form a further part of the present invention, and may be used in identifying homologues from other plant species.

Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest.

For instance, Western blotting techniques or immunoprecipitation may be used (Armitage et al, 1992, Nature 357: 80-82). Antibodies may be polyclonal or monoclonal.

Antibodies may be modified in a number of ways. Indeed the term “antibody” should be construed as covering any polypeptide having a binding domain with the required RPW specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of Chimaeric antibodies are described in EP-A-0120694 and EP-A-0125023. It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the V1 and VH domains of a single antibody; (iv) the dAb fragment (Ward, E. S. et al., Nature 341, 544-546 (1989) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P Holliger et al Proc. Natl. Acad. Sci. USA 90 6444-6448, 1993).

Candidate RPW polypeptides may be screened using these antibodies—e.g. by screening the products of an expression library created using nucleic acid derived from an plant of interest, or the products of a purification process from a natural source. A polypeptide found to bind the antibody may be isolated and then may be subject to amino acid sequencing. Any suitable technique may be used to sequence the polypeptide either wholly or partially (for instance a fragment of the polypeptide may be sequenced). Amino acid sequence information may be used in obtaining nucleic acid encoding the polypeptide, for instance by designing one or more oligonucleotides (e.g. a degenerate pool of oligonucleotides) for use as probes or primers in hybridization to candidate nucleic acid, or by searching computer sequence databases, as discussed further below.

The above description has generally been concerned with the coding parts of the RPW genes and variants and products thereof. Also embraced within the present invention are untranscribed parts of the gene.

Thus a further aspect of the invention is a nucleic acid molecule encoding the promoter of an RPW nucleic acid.

The present inventors have used northern analysis to show that transcripts for RPW8.1 and RPW8.2 increase in abundance during the resistance reaction suggesting a possible role for the promoters in transduction of the resistance signal.

Referring to the Sequence listing, the promoter of RPW8.1 is located in the region 15904 (start end) to 14719. That of RPW8.2 is within 16829 to 19087 (start end). These promoters appear to be wound and SA induced (but not JA induced).

Also embraced by the present invention is a promoter which is a mutant, derivative, or other homolog of any of the RPW promoters discussed above which has promoter activity. For instance it may be desirable to find minimal elements or motifs responsible for the resistance specific regulation. This can be done by using restriction enzymes or nucleases to digest an appropriate nucleic acid molecule, followed by an appropriate assay to determine the sequence required. Nucleic acid comprising these elements or motifs forms one part of the present invention.

“Promoter activity” is used to refer to ability to initiate transcription. The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction.

Use of a reporter gene facilitates determination of promoter activity by reference to protein production. The reporter gene preferably encodes an enzyme which catalyses a reaction which produces a detectable signal, preferably a visually detectable signal, such as a coloured product. Many examples are known, including β-galactosidase and luciferase. Those skilled in the art are well aware of a multitude of possible reporter genes and assay techniques which may be used to determine promoter activity. Any suitable reporter/assay may be used and it should be appreciated that no particular choice is essential to or a limitation of the present invention.

In a further aspect of the invention there is provided a nucleic acid construct, preferably an expression vector, including an RPW promoter region or fragment, mutant, derivative or other homolog or variant thereof having promoter activity, operably linked to a heterologous gene, e.g. a coding sequence, which is preferably not the coding sequence with which the promoter is operably linked in nature.

The invention will now be further described with reference to the following non-limiting Figures and Examples. Other embodiments of the invention will occur to those skilled in the art in the light of these.

Sequences and Figures

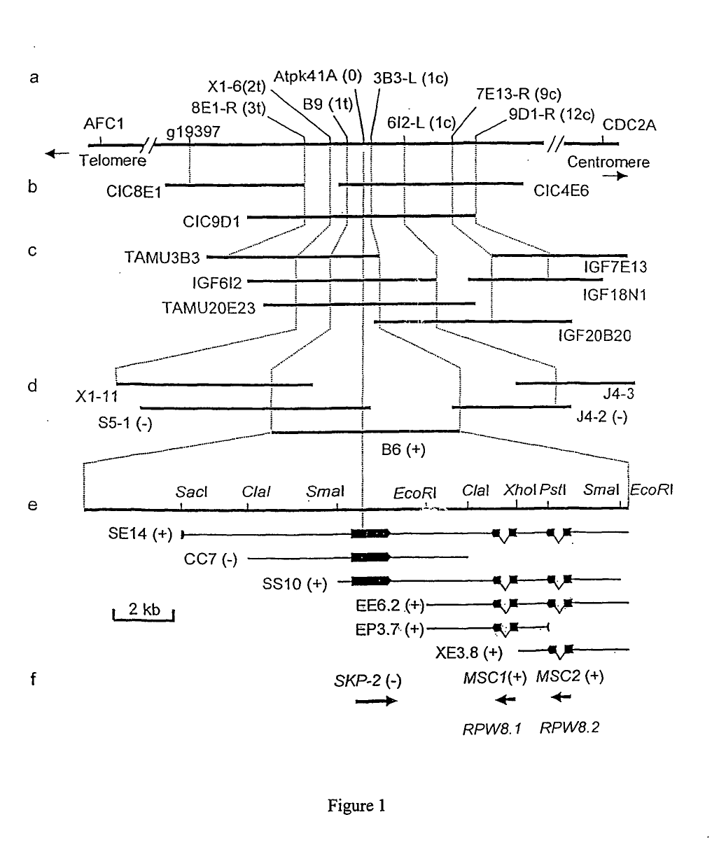

Sequence listing 1: 27689 bp contiguous genomic sequence of Arabidopsis thaliana accession Ms-0 containing RPW8.1, RPW8.2, is three RPW8 homologues HR1, HR2, HR3, and the Ms-0 homologue of SKP-2. Nucleotide locations are also given of the genomic constructs used for transformation referred to in FIG. 1e: SE14: 4160-18466; CC7: 6508-13551; SS10: 8718-18466; EE6.2: 12297-18466; EP3.7: 12297-16033; XE3.8: 14658-18466. Annotations give (where available), mRNA, coding sequence (CDS), exons, intron, transcription start, transcription end, protein sequence.

Sequence listing 2: Nucleotides 13801-18466 representing part of the DNA sequence of cosmid B6, numbered from the telomere end at marker B9 (FIG. 1a) and represents part of the sequence in Sequence listing 1. The sequence below contains only the two genes (mRNA) RPW8.1 and RPW8.2 which individually control resistance to powdery mildew caused by Erysiphe cichoracearum isolate UCSC1 and other powdery mildew pathogens. Annotations give, for each gene, the complementary nucleotides that define the transcription start (determined by 5′RACE), the first exon, the intron and the second exon (determined by comparison of genomic and cDNA sequence), and the transcription end (determined by 3′ RACE). The given protein coding sequence (CDS) sequence is the predicted amino acid translation of coding sequences in exon 1 and exon 2, for each gene.

Sequence listing 3: The cDNA nucleotide sequence of RPW8.1 from Ms-0 is aligned with that of RPW8.1 homologues isolated by PCR from other A. thaliana accessions. Accessions resistant to Erysiphs cichoracearum UCSC1 are Ms (=Ms-0), Wa (=Wa-1), Kas (=Kas-1) and C24 (=C24). Accessions susceptible to E. cichoracearum UCSC1 were Can (=Can-0), Nd (=Nd), Sy (=Sy) and Ws (=Ws-0). Nucleotide differences are in bold. In this, and other searches, parameters used were default (Blosum=62, Gap penalty=11; per residue gap cost=1; lambda ratio=0.85)/

Sequence listing 4: The predicted amino acid sequence of RPW8.1 from Ms-0 is aligned with RPW8.1 homologues isolated by PCR from other A. thaliana accessions. Dash (-) indicates identity with the RPW8.1/Ms-0 sequence; dot (.) indicates gap, or no equivalent sequence. Single letter codes beneath the Ms-0 sequence indicate predicted differences. Accessions resistant to Erysiphs cichoracearum UCSC1 are Ms (=Ms-0), Wa (=Wa-1), Kas (=Kas-1) and C24 (=C24). Accessions susceptible to E. cichoracearum UCSC1 were Can (=Can-0), Nd (=Nd), Sy (=Sy) and Ws (=Ws-0). The analysis shows that the predicted amino acid sequence at RPW8.1 of the resistant accessions is identical to that of accession Ms-0. Susceptible accessions have amino acids different from the resistant accessions at one or more of the following positions: 31, 33, 40, 43, 45, 77, 95, 108, an insertion at 121-141, and at 169.

Sequence listing 5: The cDNA nucleotide sequence of RPW8.2 from Ms-0 is aligned with that of RPW8.2 homologues isolated by PCR from other A. thaliana accessions. Accessions resistant to Erysiphs cichoracearum UCSC1 are Ms (=Ms-0), Wa (=Wa-1), Kas (=Kas-1) and C24 (=C24). Accessions susceptible to E. cichoracearum UCSC1 were Can (=Can-0), Nd (=Nd), Sy (=Sy) and Ws (=Ws-0). Nucleotide differences are in bold. Stop codons are in italics.

Sequence listing 6: The predicted amino acid sequence of RPW8.2 from Ms-0 is aligned with RPW8.2 homologues isolated by PCR from other A. thaliana accessions. Dash (-) indicates identity with the RPW8.1/Ms-0 sequence; dot (.) indicates gap, or no equivalent sequence. Single letter codes beneath the Ms-0 sequence indicate predicted differences. Accessions resistant to Erysiphs cichoracearum UCSC1 are Ms (=Ms-0), Wa (=Wa-1), Kas (=Kas-1) and C24 (=C24). Accessions susceptible to E. cichoracearum UCSC1 were Can (=Can-0), Nd (=Nd), Sy (=Sy) and Ws (=Ws-0). The analysis shows that the predicted amino acid sequence at RPW8.2 of the resistant accessions is identical to that of accession Ms-0. Susceptible accessions have amino acids different from the resistant accessions at one or more of the following positions: 17, 19, 64, 70, 111, 116, termination at 144, and 161. It is notable that accession C24, which is resistant, has an RPW8.2 sequence different to that of the other resistant accessions, whereas the RPW8.1 amino acid sequence is identical to that of the resistant accessions (see Sequence listing 4).

Sequence listings 7-9: sequences for BrHR1—genomic DNA, predicted is cDNA, and predicted encoded amino acid respectively.

Sequence listings 10-12: sequences for BrHR2—genomic DNA, predicted cDNA, and predicted encoded amino acid respectively.

Sequence listings 13-15: sequences for BrHR3—genomic DNA, predicted cDNA, and predicted encoded amino acid respectively.

FIG. 1: Map-based cloning of RPW8. [a] Order of molecular markers used in the fine-mapping of RPW8. Vertical broken lines link the genetic location of markers to their physical position on subcloned DNA; sequence identity was demonstrated by hybridisation. Figures in brackets are the numbers of plants with recombinations between RPW8 and the indicated marker closer to the telomere (t) or centromere (c). [b, c] Aligned A thaliana Col-0 genomic DNA from (b) VAC and (c) 8AC clones which hybridised to the indicated molecular markers genetically linked to RPW8. [d] Alignment of cloned A. thaliana Ms-0 genomic DNA which hybridised to the indicated genetic markers. “(+)” indicates that Col-O plants transformed with the DNA were resistant, and “(−)” indicates they were susceptible, to E. cichoracearum UCSC1. [e] Restriction sites of cosmid 86 used for sub-cloning A. thaliana Ms-0 DNA. ORFs detected in the B6 sequence are shown as thick lines in the subclones. Subclones are marked (+) and (−) as for (d) f. Aligned Ms-D cDNAs. Cloned cDNAs expressed under a constitutive viral promoter are marked (+) and (−) as for (d).

FIG. 2. Analysis of the RPW8locus. [a] The RPW8 locus consisted of five tandemly linked homologues. [b, c] Predicted amino acid sequences of (a) RPW8.1 and (b) RPW8.2 from accession Ms-O. Sequences in italics are predicted to form transmembrane (TM) domains, or possibly signal peptides. Sequences in bold are predicted to form coiled coils (CC). Lowercase letters above the sequence indicate the heptad repeats that define coiled coils in which ‘a’ and ‘d’ are typically hydrophobic, while the other residues tend to be hydrophilic.

FIG. 3. The SE7.5 plant transformation vector described in Example 8.

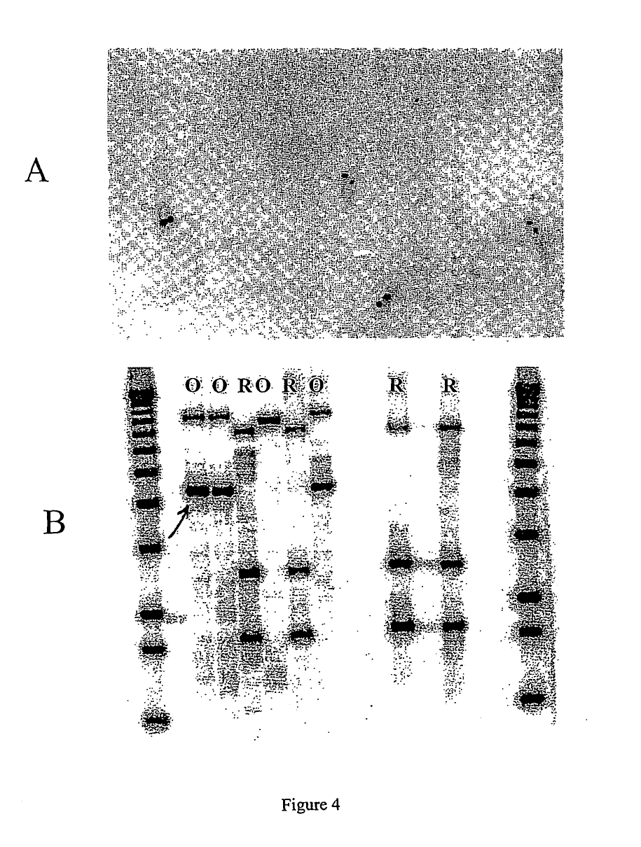

FIG. 4. Identification of RPW8 homologs in Brassica rapa (R) and B. oleracea (O).

A. One BAC filter B. rapa probed with AtRPW8.1 & 2 and AtHR1-3 sequentially, showing the same clones hybridised to the probes.

B. DNA from positive BAC clones was digested with EcoRI and BamHI, separated in agarose gel, blotted to membrane, and probed to the DNA mixtures as in A.

### EXAMPLES

### Example 1

### Localisation of RPW8

Plasmid B6

A. thaliana accession Col-0 is susceptible and accession Ms-0 is resistant to infection by E. cichoracearum UCSC1.

This was confirmed by observation 10 days after inoculation (results not shown) after which time Col-0 supported growth of superficial white mycelium whereas Ms-0 did not. Resistance of accession Ms-0 is controlled by the RPW8 locus which maps genetically to an 8.5 cM interval, flanked by markers CDC2A and AFC1(8) (FIG. 1a). A population of 1,500 F₂ plants from a cross between accessions Ms-0 and Ler (susceptible to E. cichoracearum UCSC1) was screened to detect individuals with recombination break points between markers g19397 and CDC2A (FIG. 1a). The ninety-four recombinants recovered were used to fine-map RPW8. Their genotypes at the RPW8 locus were deduced by scoring F₃ progeny for resistance or susceptibility to E. cichoracearum UCSC1. Their genotypes at selected RFLP markers in the g19397 and CDC2A intervalrevealed that Atpk41A co-segregated with RPW8, and that YAC ends 8E1-R and 9D1-R flanked the RPW8 locus (FIG. 1b). Atpk41A, 8E1-R and 9D1-R were used as hybridisation probes to screen the TAMU and IGF BAC libraries, which were re-screened with BAC ends from some of the recovered clones. Five of the isolated clones formed a ˜200 kb contiguous, overlapping series of that spanned RPW8 (FIG. 1c). A genomic library of the resistant accession Ms-0 was constructed and screened for clones that hybridised to markers 8E1-R, Atpk41A, 6I2-L, and 3B3-L. Five recovered clones formed a 45 kb contiguous series that spanned the RPW8 locus (FIG. 2d). RPW8 was flanked by genetic markers B9 and 3B3-L, which were both located in cosmid B6 (FIG. 1d). The B6 insert was introduced into the powdery mildew-susceptible accession Col-0 by Agrobacterium-mediated transformation(10). The transformed progeny, represented here by T-B6, were resistant to infection by E. cichoracearum UCSC1(results nor shown) whereas plants transformed with cosmids S5-1 and J4-2 were susceptible (not shown). This confirmed that cosmid B6 contained RPW8.

To localise RPW8, cosmid B6 was sequenced and a variety of fragments of cosmid B6 were sub-cloned in a plant transformation vector and introduced into Col-0 plants by Agrobacterium-mediated transformation (FIG. 1e). The DNA sequence of B6 revealed three ORFs.

One had similarity (predicted amino acid sequence identity was 100%) to the cDNA ATHPROKINA (GenBank Accession L05561) from which marker Atpk41A was derived, and which corresponds to the gene protein kinase SPK-2 (GenBank Accession S56718) located in BAC T20E23 (GenBank Accession AL133363) from A. thaliana accession Col-0 (FIG. 1c). We therefore named this ORF SPK-2/M to denote it as the Ms-0 allele of SPK-2.

ORFs MSC1, and MSC2 had no obvious alleles in the A. thaliana Col-0 sequence in T20E23 (FIG. 1f).

Plants transgenic for subclones SE14, SS10, EE6.2, EP3.7 and XE3.8 were resistant to E. cichoracearum UCSC1, whereas plants transgenic for subclone CC7 were susceptible (FIG. 1e, Sequence listing 1 gives the sequence for these fragments). The subclones that conferred resistance contained either ORF MSC1 (EP3.7), or ORF MSC2 (XE3.8), or both of these (FIG. 1e). This indicated that RPW8 comprised two independently-acting genes, MSC1 and MSC2, which were therefore re-named RPW8.1 and RPW8.2 respectively. The entire 18466 nt B6 sequence, containing SKP-2/M, RPW8.1 and RPW8.2, and part of the contiguous sequence of cosmid J2-4 (FIG. 1e) is given in Sequence listing 1.

cDNAs for RPW8.1 and RPW8.2, and for SKP-2/M as control, were cloned into a plant transformation vector under control of the highly active cauliflower mosaic virus 35S promoter, and introduced into Col-0 plants by Agrobacterium-mediated transformation. Transgenic plants T-35s::RPW8.1 and T-35s::RPW8.2, were resistant to E. cichoracearum UCSC1 whereas the transgenic plants T-35s::SKP-2 were susceptible (results not shown). We concluded that RPW8 contains two functional genes, RPW8.1 and RPW8.2, which are each sufficient for resistance to E. cichoracearum UCSC1. Sequence listing 2 gives the 4665 nucleotide sequence of A. thaliana accession Ms-0 DNA which contains the predicted promoters and the transcribed sequences of RPW8.1 and RPW8.2.

### Example 2

### Characterisation of Specificity Controlled by RPW8

A range of pathogens virulent on A. thaliana accession Col-0 were used to characterise the specificity of resistance controlled by RPW8. Transgenic plants T-B6, T-35s::RPW8.1 and T-35s::RPW8.2 were resistant to all of the tested powdery mildew pathogens. These included 15 isolates of E. cichoracearum, and E. cruciferarum isolate UEA1, E. orontii isolate MGH, and Oidium lycopersici isolate Oxford, representing four distinct species(11). These results indicate that RPW7, which controls resistance to E. cruciferarum UEA1 and maps with RPW8 between markers CDC2A and AFC1(8) (FIG. 1a), may be identical to RPW8.1 and RPW8.2. Significantly, T-B6 plants were susceptible to other pathogens, including the fungus Peronospora parasitica Noco2 to which Ms-0 was resistant (testing 7 days after inoculation for white sporagiophores, results not shown), the cauliflower mosaic virus, and to the bacterium Pseudomonas syringae pv tomato DC3000 (results not shown). Because none of the powdery mildew pathogens we have tested could infect plants containing the RPW8 locus, we have no formal evidence of a gene-for-gene interaction. RPW8.1 and RPW8.2 defined in Sequence listing 2 appear to represent a special type of R-gene which controls “specific” resistance to a broad group of the powdery mildew pathogens.

### Example 3

### RPW8 Homologues from other Accessions

RPW8.1 produced a 908 nt transcript with a single 197 nt intron and 444 nt of predicted coding sequence, and RPW8.2 produced a 926 nt transcript with a 128 intron and 522 of predicted coding sequence (Sequence listing 1 & 2).

We examined the sequences of RPW8.1 and RPW8.2 homologues in seven other A. thaliana accessions with different levels of resistance to E. cichoracearum UCSC1. Accessions Kas-1 and Wa-1 are strongly resistant(12, 13), and a major resistance gene in each has been genetically mapped to the RPW8 locus (S. Somerville, personal communication). RPW8.1 and RPW8.2 homologues were amplified from Kas-1 and Wa-1 by PCR, and the DNA sequences were identical to those of the corresponding Ms-0 genes in Sequence listing 2. RPW8.1 and RPW8.2 homologues were also amplified by PCR from the moderately susceptible accessions, Ler, Nd-0, and Ws-0. Their derived amino acid sequences differed from those of the corresponding Ms-0 genes by 1.1-4.1%. We could not detect either an RPW8.1 or an RPW8.2 homologue in the extremely mildew-susceptible accession Col-0(13), by Southern analysis (results not shown), PCR, or by inspection of the published sequence at the RPW8 locus in Col-0 (BAC 20E23, FIG. 1c). Resistance of A. thaliana to E. cichoracearum UCSC1 in these A. thaliana accessions is therefore associated with extreme conservation of DNA sequence at RPW8.1 and RPW8.2.

### Example 4

### RPW8 Homologues on Cosmid B6

Southern blotting showed that RPW8.1 and RPW8.2 were present in Ms-0 and Kas-1 as single-copy genes (not shown). However, the nucleotide sequence of cosmid B6 and J4-2 (FIG. 1c, Sequence listing 1) revealed that RPW8 was linked to three ORFs with 55.0-64.2% DNA sequence identity to RPW8.1 and RPW8.2. These were named Homologous to RPW81 (HR1), HR2, and HR3, and they were also closely related (99.4-99.9% DNA sequence identity) to predicted genes CAB62476, -5 and -4, respectively, in BAC 20E23 from accession Col-0. A recombination break-point between RPW8.2 and HR3, detected with marker 3B3-L (FIG. 1a) indicated that HR1, -2, and -3 were not required for resistance to powdery mildew. The RPW8 locus of Ms-0 therefore contains five tandemly arranged RPW8 homologues (FIG. 2a, annotated also in Sequence listing 1), three of which are also represented in Col-0. Other R gene-loci also contain clusters of homologues(14), members of which may recognise different strains of the pathogen(15). These gene clusters apparently evolve new R-gene specificities rapidly, through duplication, unequal crossover and mutation(16, 17). A comparison of HR1, -2, -3, RPW8.1, and -2 by PILEUP, below, shows regions of similarity between the predicted proteins.

| | |
|---|---|
| {hr1} | MPvsEimaGA ALGLaLQvLH dAikkAKDrS ltTrcILdRL dATIfrITPl |
| {hr2} | MPltEiiaGA ALGLaLQiLH eAiqrAKDrS ltTscILdRL dsTIlrITPl |
| {hr3} | MP1vElltsA ALGLsLQlLH eAiirAKekt liTrcILdRL dATlhkITPf |
| {rpw82} | ˜miaEvaaGg ALGLaLsvLH eAvkrAKDrS vtTrfILhRL eATIdsITPl |
| {rpw81} | MPigElaiGA vLGvgaQaiy drfrkArDiS .....fvhRL cATIlsIePf |
| Consens | MP--E---GA ALGL-LQ-LH -A---AKD-S --T--IL-RL -ATI---ITP- |
| | 51 100 |
| {hr1} | vtqvDKlseE vedSp.RKVi EdLKhLLEkA vsLVEAYAEL rRRNlLkKfR |
| {hr2} | makveKlnkE sdeSl.RKVf EdLKhLLEkA vvLVEAYAEL kRRNlLeKyR |
| {hr3} | vikiDtlteE vdepf.RKVi EeLKrLLEkA irLVdAYAEL klRNlLrKyR |
| {rpw82} | vvqiDKfseE medStsRKVn krLKlLLEnA vsLVEenAEL rRRNvrkKfR |
| {rpw81} | lvqiDKrsk. vegSplReVn ErLtcfLElA yvfVEAYpkL rRRqvLrKyR |
| Consens | ----DK---E ---S--RKV- E-LK-LLE-A --LVEAYAEL -RRN-L-K-R |
| | 101 150 |
| {hr1} | YkRrIKElEa sLRWmvDVDV QVNQWvDIKe LmAKMSEMnT KLdeItrQP. |
| {hr2} | YkRrIKElEg sLkWmvDVDV kVNQWaDIKd LmAKMSENnT KLekImgQP. |
| {hr3} | YkRrIKElds sLRWmiDVDV QVNQWlDIKk LmgKMSEMnT KLddItrQP. |
| {rpw82} | YmRdIKEfEa kLRWvvDVDV QVNQlaDIKe LkAKMSEisT KLdkImpQPk |
| {rpw8l} | YikaIetiEl aLRsiivVDf QVdQWdDIKe ikAKiSEMdT KLaevisacs |
| Consens | Y-R-IKE-E- -LRW--DVDV QVNQW-DIK- L-AKMSEM-T KL--I--QP- |
| | 151 200 |
| {hr1} | tdcicfksnh stsqsssqni veetdrslee ivecssdgsk pkidihihws |
| {hr2} | idciisedn. .....tnmdi vervdpslea kagcsnsdsk pkidihlrws |
| {hr3} | .......... .......mdi ieatgrssee d.gc....tk ptidihfrw. |
| {rpw82} | feihigwcsg ktnrairftf csdds˜˜˜˜˜˜˜˜˜˜˜˜˜˜˜˜˜˜˜˜˜˜˜˜˜ |
| {rpw81} | kira˜˜˜˜˜˜˜˜˜˜˜˜˜˜˜˜˜˜˜˜˜˜˜˜˜˜˜˜˜˜˜˜˜˜˜˜˜˜˜˜˜˜˜˜˜˜ |
| Consens | ---------- ---------- ---------- ---------- ---------- |
| | 201 215 |
| {hr1} | srkrnkdrei rfvlk |
| {hr2} | ..kqskdhgi rfvln |
| {hr3} | .knqtkehei rfifk |
| {rpw82} | ˜˜˜˜˜˜˜˜˜˜˜˜˜˜˜ |
| {rpw81} | ˜˜˜˜˜˜˜˜˜˜˜˜˜˜˜ |
| Consens | ---------- ----- |

HR1, -2, and -3 may therefore represent R-genes with as-yet unknown specificity. BLAST searches with these peptides show no obvious similarity to any other characterised gene products, however. This suggests that the RPW8 proteins represent a novel type of protein.

### Example 5

### Northern Analysis

Northern analysis indicated that transcripts for RPW8.1 and RPW8.2 of the appropriate size were expressed in uninoculated T-B6 plants, but that transcript levels increased in abundance during the resistance reaction (not shown).

### Example 6

### Structure of RPW8.1 and 8.2

The predicted RPW8.1 and RPW8.2 proteins have 45.2% sequence identity, and are relatively small (molecular weights 17,000 and 19,973, respectively) and basic (pIs of 9.46 and 10.05, respectively). RPW8.1 and RPW8.2 had no significant similarity to the derived proteins from other R-genes, nor to any characterised plant gene. Analysis of the RPW8 sequences indicated a predicted N-terminal TM domain, or possibly a cleavage signal peptide, and a CC domain (FIGS. 2b & c). Therefore RPW8 defines a new class of R-gene product, which we name here TM-CC.

### Example 7

### RPW8 Homologues from other Plant Species

Barley

In the light of the present disclosure, those skilled in the art will appreciate that RPW8 homologues may be isolated from barley by any of several techniques.

A preferred method is to identify clones in a genomic library of barley that hybridise to DNA for RPW8.1 and/or RPW8.2 as follows.

A cosmid library representing the barley genome might contain 80,000 clones each with an insert size of 20-40 kb. These are stored individually. DNA from each clone is isolated, and 40 pools are made, each containing DNA from 2,000 clones. Samples from the pools can be digested with EcoRI, or another suitable enzyme which releases the vector sequence. Digested DNA samples are run out on 1% TAE agarose gels. The DNA in the gels is treated with standard depurination, denaturation and neutralisation buffer (Sambrook et al., 1989) before overnight capillary blotting onto Hybond N⁺ (Amersham) membrane with 10×SSC, and fixation at 80° C. for 2 hours. DNA representing the coding region of RPW8.1 and RPW8.2 is amplified from Arabidopsis thaliana accession Ms-0 cosmid B6 by PCR with specific primers (such as GACCCGTACAGTACTAAGTCTA and GATTTCCGAAATTGATTACAAGAA for RPW8.1, and for RPW8.2, the primers AACTCTTCACCTCGAGAGCTAACA and AGTCGTTTGACACAATTGGGACAT), labelled with ³²P-dCTP with a Multiprime DNA labelling kit (Amersham) according to the manufacturer's instructions. Blots are washed 2-3 times in 2×SSC, 0.1% SDS (low stringency wash) at 65° C. and, if necessary, in 0.2% SSC, 0.1% SDS (high stringency wash). Hybridisation signal is detected by phosphor screens scanned in a Storm 840 phosphor imager (Molecular Dynamics).

Pools that reveal bands where digested DNA has hybridised to the probe DNA are then reconstituted as 40 sub-pools, each containing the DNA from 50 clones. The sub-pools are screened again with probes RPW8.1 and RPW8.2, as described for the pools, and sub-pools that reveal hybridising bands are identified. The chosen sub-pools are now represented as DNA samples from each of the 50 constituent clones, and these are screened as for the pools, to identify the genomic clone that gave rise to an hybridising band in the pool, and in the sub-pool.

The process will therefore identify one or more clones of genomic DNA from barley that contain sequences homologous to the RPW8 genes. To precisely identify the barley DNA sequence that is homologous to RPW8, an efficient approach is to subclone the cosmid as 2-5 kb fragments in a chosen vector, such as Bluescript. Subclones are then screened as colony blots according to the manufacturer's instructions of the nitrocellulose membrane, using 32-P-labelled RPW8.1 and RPW8.2 as probe. Individual subclones that hybridize to the probes are recovered, and the cloned DNA is sequenced. Software programs such as Blast, and PileUp are used to locate regions in the subcloned DNA with similarity to RPW8.1 and RPW8.2.

Brassica napus

Southern analysis was used to determine if sequences in Brassica napus hybridized to RPW8.1 and RPW8.2. It was found that B. napus does contain DNA which hybridized to RPW8.1 and RPW8.2, suggesting that RPW8 homologues occur in this species.

Southern Analysis of Brassica napus

DNA was extracted from leaves of B. napus as follows. Approximately 3 g of leaves were ground into powder with liquid nitrogen in a pre-chilled mortar. The powder was transferred to a 50 ml centrifuge tube and carefully mixed with 20 ml of urea extraction buffer (8 M urea, 50 mM Tris pH8, 20 mM EDTA pH 8, 350 mM NaCl, 2% sarcosine and 5% phenol). 800 microlitres 20% SDS was added and the mixture was incubated at 65° C. for 10 min. The solution was extracted with phenol/chloroform (1:1), centrifuged at 2,000 g and the aqueous phase was extracted again with phenol/chloroform (1:1), maintained at 4° C. for 20 min, 4 ml 5 mM potassium acetate was added, the samples gently mixed and held on ice for 30 min. The debris was spun down at 2,000 g, 4° C. for 20 min, and 16 ml isopropanol was gently mixed into the supernatant. The DNA was immediately pelleted at 2000 g for 15 min. The pellets were dried and then dissolved in 1 ml TE.

DNA was digested overnight with the restriction enzyme EcoRI. Digested DNA samples were separated on 1% TAE agarose gels. DNA in the gels was depurinated, denatured and neutralised (Sambrook et al., 1989), transferred to Hybond N⁺ (Amersham, UK) nylon membranes by capillary blotting overnight with 10×SSC, and fixed to the membrane at 80° C. for 2 hours.

RPW8.1 and RPW8.2 were amplified by PCR from genomic DNA of Arabidopsis thaliana accession Ms-0, using as primers the sequences designed to against the beginning and the end of the predicted coding sequences. The amplified products were labelled with ³²P-dCTP using a Multiprime DNA labelling kit (Amersham, UK) according to the manufacturer's instructions. Blots were washed 2-3 times in 2×SSC, 0.1% SDS (low stringency wash) at 65° C. and, if necessary, in 0.2% SSC, 0.1% SDS (high stringency wash). Hybridisation was detected by phosphor screens scanned in a Storm 840 phosphor imager (Molecular Dynamics, USA).

Two bands, 4 kb and 1 kb, could be distinguished in the lanes for B. napus.

Brassica rapa and B. oleracea

BAC libraries of Brassica rapa (B. rapa ssp oleifera cv R018) of B. oleracea (B.oleracea ssp. alboglabra cv A12) were constructed.

These libraries were screened using a mixture of AtRPW8.1 and AtRPW8.2 genomic DNA (amplified with AtRPW8-specific primers described above with 6I2B6 cosmid DNA as template) as probe, with 50 ng of each PCR product being mixed and used for the labelling with dCTP³².

Hybridisation was carried out at 50° C. overnight, and the filters were washed at 50° C. with 2×SSC and 0.1% SDS solution three time. Eighteen BAC clones from B. rapa and 7 clones B. oleracea from were identified hybridising to AtRPW8.

A second hybridisation with the same filters under the same conditions mentioned above using as probe the DNA mixture of AtHR1, AtHR2 and AtHR3 (these were each amplified by primers corresponding to the first 24 bp and last 24 bp of the predicted coding sequences of these three homologs—˜30 ng DNA from each was mixed for labelling) indicated the same BAC clones also hybridised with the AtHR genes (see FIG. 4).

Fingerprinting was performed according to the manufacturer's instructions as follows: the DNA of all the BAC clones was digested with EcoRI and BamHI, separated on 0.8% agarose gel, and then photographed under UV light following hybridisation. For the AtHR1-3 DNA probes the digested DNA in the gel was blotted to a Nitrocellulose membrane purchased from Roche. The blots were sequentially probed with dCTP³²-labelled AtRPW8 and AtHR1-3 DNA mixtures described above under the same conditions

Fingerprinting revealed that the genomes of both B. rapa and B. oleracea contain a single RPW8-like locus since all the positive BAC clones from either B. rapa or B. oleracea formed only one overlapping contig, as does the Arabidopsis genome (FIG. 2). Subcloning and sequencing was first performed with one positive BAC clone from B. rapa.

We found the B. rapa genome contains three RPW8-like genes tandemly linked with each other. The sequence of the genomic DNA, the predicted cDNA and deduced amino acids was listed in the sequence listing.

These three genes (named BrHR1, BrHR2, and BrHR3) are highly homologous to each other (83-97% at amino acid level) and show 44-74% amino acid identity to AtRPW8.1, AtRPW8.2 and AtHR1-3.

Further results have shown that B. oleracea genome also contains three RPW8-like sequences (named BoHR1, BoHR2, and BoHR3), and the organisation of these genes is very similar to that of the B. rapa homologs.

It is clear that these three genes are the Brassica homologs of AtRPW8 genes. Thus the AtRPW8.1 and AtRPW8.2 genomic DNA hybridises to the 3 Brassica sequences, so does the AtHR1, AtHR2 and AtHR3 genomic DNA. Secondly, BLAST search shows that these 3 sequences only pick AtRPW8 and its homologs, and they show considerably high homology to the AtRPW8 family members. Thirdly, these 3 homologs are highly homologous to each other, and to AtHR3, implying they share a common origin.

Expression of the genes may be conformed by RT-PCR, while their resistance function can be confirmed by putting them under the control of AtRPW8 promoter(s) and introducing them into Arabidopsis Col-0 background which is then challenged by the same pathogens discussed above.

### Example 8

### Introduction of RPW8 Into Plant Species

Transformation of Nicotiana benthamiana with Cosmid B6

RPW8 was transferred to Nicotiana benthamiana by stable, Agrobacterium mediated transformation of N. benthamiana plants with cosmid B6. Rather surprisingly, the transgenic plants were resistant to E. cichoracearum. This indicated that RPW8 functioned in the heterologous host, N. benthamiana.

N. benthamiana transformations were based upon the leaf disc method of Horsch et al. (1985) and Horsch and Klee (1986). Leaves approximately 90 mm wide were removed from young plants approximately 10 cm in height and surface-sterilised by immersion in 2% bleach for 15 minutes, followed by one rinse in 70% ethanol and five 10-minute washes in sterile water. Discs of 0.5 cm diameter were punched from the leaves using a flame-sterilised size 3 cork borer incubated on pre-callusing plates (Horsch et al. (1985)) in continuous light for 24 hours at 22° C. The leaf discs were then dipped in an overnight LB culture of Agrobacterium tumefaciens strain GV3101 (O.D. 600=0.1) containing the B6 cosmid, transferred to fresh pre-callusing plates, and returned to the growth chamber. After 48 hours the discs were transferred to selection media, containing phospinothricin (PPT) at 5 mg per litre, and carbenicillin at 500 mg per litre to kill the Agrobacterium. Transformed explants produced green shoots after 3-5 weeks that were excised using a flame-sterilised scalpel and transferred to Magenta pots containing rooting media (Horsch et al. (1985). Upon rooting, shoots were grown in moistened, sterilised soil comprising John Innes No.3 compost, coarse grit, peat and vermiculite. Pots were placed in glass jars and covered with transparent film for 3-4 days to retain high humidity. Plants were then maintained at 23° C. under short day conditions to delay flowering.

Twelve transgenic Nicotiana benthamiana plants (T1-T12) were regenerated from a screen of 28 leaf disc explants.

Single leaves from the putative transgenic tobacco plants were sprayed with three applications of 30 mg per litre BASTA herbicide containing glufosinate ammonium were sprayed over 6 days. All were resistant to BASTA, confirming that they had been transformed.

Results of N. benthamiana Transformed with B6

The transgenic plants T5 and T6 and plants transformed with vector only, as control, were inoculated with E. cichoracearum UCSC1. T5 and T6 plants were resistant to E. cichoracearum, whereas the controls were susceptible, and the fungus grew as a superficial white mycelium clearly visible to the naked eye.

N. benthamiana Transformation with SE7.5

The SE7.5 construct was made as follows: A 7.5 kb SmaI and EcoRI fragment starting 1637 bp upstream of RPW8.2 (SmaI site of B6 cosmid clone in the SLJ755I5 vector obtained from the JIC) and ending 2912 bp downstream of RPW8.1 (EcoRI site inside the ATPK41A gene) was obtained by SmaI complete digestion of first and then partial EcoRI digestion of the B6 cosmid clone. The 7.5 kb fragment was recovered, purified, and ligated to SamI-EcoRI digested pBIN19-Plus binary vector (obtained from JIC). The resulting plasmid carryied AtRPW8.1 and AtRPW8.2 genomic sequence including their native promoters and was named SE7.5 (see FIG. 3). It was introduced to E coli (DH10B from GIBCOL-BRL).

Agrobacterium (strain GV3101, obtained from The Sainsbury Lab, JIC) was used for transformation. The Agrobacterium strain was grown for 48 hours at 30° C. in 10 ml LB medium supplemented with 25 μg/ml Rifampicin, 25 μg/ml Gentamycin, 50 μg/ml Kanamycin. About 100 μl of this cell culture was then added to 10 ml fresh LB medium without antibiotics, and shaken for further 24 hours at 30° C. The Agrobacterium was then diluted with liquid MS medium to achieve an OD₆₀₀ of 0.1.

Tobacco leaves from young plants were surface-sterilized by immersion in 2% bleach (12% active Cl⁻w/v) followed by one rinse in 70% ethanol and five 10 minute-washes in sterile water. Discs of 0.5 cm. diameter were punched from the leaves using a flame-sterilized size 3 cork borer. The leaf discs were incubated on regeneration plates, sealed with micropore tape and kept under continuous light for 24 hours at 22° C. in a growth cabinet. The leaf discs were then dipped in the diluted Agrobacterium, swirling occasionally. Excess liquid was removed with filter paper and leaf discs were transferred to fresh regeneration media, sealed and returned to the growth cabinet. After two days co-cultivation at 22° C., the tobacco leaf discs were transferred to selective regeneration medium containing 500 mg/L Carbenicillin and 100 mg/L Kanamycin as selective agents (about 10 leaf discs per 9 cm-diameter petri dish) and cultured at 22° C. under continuous light. Transformed explants produced green shoots after 3-5 weeks which were excised and placed on rooting medium containing 200 mg/L Carbenicillin, and 100 mg/L Kanamycin in sealed glass jars (Magenta pots). Rooting plants were transferred and grown in moistened, sterilized soil. Plants were maintained in a sealed propagation tray to retain high humidity under short day condition for a number of days, then transferred to normal humidity conditions in the glasshouse.

About 4 weeks old transgenic T1 plants and wild type plants were inoculated with Erysiphe cichoracearum UCSC1, and their phenotypes were checked 10 days after inoculation. Erysiphe cichoracearum UCSC1 was obtained from the Carnegie Institute, Washington, Stanford USA, where it was originally identified on Arabidopsis Col-0 plants grown in their greenhouse, and was subsequently purified from a single colony.

Results of N. benthamiana Transformation with SE7.5

More than 20 T1 lines of transgenic N. benthamiana were generated. The presence of AtRPW8 genes was confirmed by PCR using AtRPW8.1-specific primers (5′- ATGCCGATTGGTGAGCTTGCGATA-3′ and a reverse, 5′-TCAAGCTCTTATTTTACTACAAGC-31). and AtRPW8.2-specific primers (5′-ATGATTGCTGAGGTTGCCGCA-3′ and 5′-TCAAGAATCATCACTGCAGAACGT-3′).

T2 progenies of 5 T1 lines were tested with UCSC1 isolate, which is the only isolate we found that can mildly infect N. benthamiana. All the 5 lines showed no or very little fungal growth (disease rating: 0, or 0-1) and some T2 plants developed obvious necrotic lesions (HR), whereas, the wild type plants supported more fungal growth and sporulation (disease rating:1, or 1-2), and had no obvious necrotic lesions.

Nicotiana tabacum Transformation with SE7.5

N. tabacum variety Petit Gerard was used for transformation. The transformation procedures were the same as that used for N. benthamiana, except that axenic tobacco leaves were used as explants and A. tumefaciens strain LBA4404 containing SE7.5 construct was used for transformation.

Results of Nicotiana tabacum Transformation with SE7.5

Eighteen T1 lines carrying both AtRPW8.1 and AtRPW8.2 genes were generated. And 4 of them were tested with Erysiphe orontii MGH (originally identified and purified on Arabidopsis plants by Dr. Fred Ausubel's group in Massachusetts General Hospital, Harvard University) along with the wild type. The wild type N. tobaccum plants were fully susceptible to this isolate (disease rating 2-3 or 3), while three T1 plants were completely resistant (no visible fungus; disease rating 0) and surprisingly, had no visible necrotic lesions. One T1 plant supported a little fungal growth (disease rating 0-1˜1) and had some necrotic lesions.

Transfer of RPW8 to Wheat, Barley and to Tomato.

Those skilled in the art are well aware of methods for the production of stable, fertile transgenic plants of Triticum aestivum (wheat), Hordeum sativum (barley), and Lycopersicon esculentum (tomato) by Agrobacterium-mediated transformation and transfer of RPW8.1 and RPW8.2 to wheat, barley, and tomato can be achieved using any preferred methods. Transgenic plants so produced can be tested for resistance to powdery mildew pathogens that affect the relevant crop species.

Generally speaking, RPW8.1 and RPW8.2 may be amplified by the primers specified such as GACCCGTACAGTACTAAGTCTA and GATTTCCGAAATTGATTACAAGAA (for RPW8.1) and AACTCTTCACCTCGAGAGCTAACA and AGTCGTTTGACACAATTGGGACAT (for RPW8.2) and cloned into a binary vector, introduced into the specified strain of Agrobacterium tumefaciens by electroporation, and used to transform appropriate tissue from the different plant species.

Transformation of Tomato with RPW8.1 and RPW8.2:

One suitable method employs A. tumefaciens strain LBA4404 with the binary vector of Filliati (1987). Tomato seeds are germinated under sterile conditions, and cotyledon explants are placed on filter paper on tobacco cell feeder cultures and co-cultivated with A. tumefaciens as specified in Filliati (1987) and McCormick (1986). Selection is applied with kanamycin (McCormick, 1987), and shoots that develop are transferred to rooting medium, and then to soil. Tests for the transgene (McDonnell, 1987) are used to confirm transgenic plants. These are grown on to collect seed. Progeny from these primary transgenic plants are then tested for resistance to powdery mildew.

For example, Lycopersicon esculentum transformation with SE7.5 may be carried out as follows:

Preparation of tomato seedlings: Tomato variety Moneymaker was used for transformation. tomato seeds were treated with 70% ethanol for 2 minutes and rinsed once with sterile water. Then, the seeds were immersed in 10% Domestos and shaken for 3 hours followed by 4 times of washes with water. The seeds were left in water and shaken at 25° C. overnight. About 25 seeds were put into tubs containing germination medium and left in 4° C. for 2 weeks. Seedlings were grown under continuous light at 22° C. growth cabinet for 7-10 days.

Preparation of Agrobacterium culture: A. tumefaciens strain LBA4404 containing SE7.5 construct was used for transformation. The strain was inoculated to 10 ml LB containing 25 μg/ml Rifampicin, 25 μg/ml Gentamycin, 50 μg/ml Kanamycin and the cultured in a 28° C. shaker for 28 hours.

Setting up feeder layers: 1 ml of fine tobacco suspension culture was spread evenly onto plates containing MS medium with 0.5 mg/L 2,4-D, 0.6% agar. The plates were left unsealed and stacked, and put under continuous light at 22° C. growth cabinet for 24 hours.

Incubation of explants: A piece of Whatman no.1 filter paper was placed on top of the feeder plate and wet completely. Any air bubbles were excluded. Young and still expanding cotyledons of tomato seedlings prior to true leaf formation were used as explants. Cotyledon tips were cut off and two more transverse cuts were made to give two explants of about 0.5 cm. long. The explants were transferred to a new petri dish full of water to prevent any damage during further cutting. Once a number of explants were collected in the pool, they were dabbed onto sterile filter paper and 30-40 explants were then placed onto a feeder plate, with topside down. Petri dishes were placed unsealed and stacked under continuous light at 22° C. growth cabinet for 8 hours.

Co-cultivation: Agrobacterium cells were spun down and resuspended in MS medium containing 3% sucrose to an OD₅₉₀ of 0.4-0.5. The explants from feeder plates were completely immersed in bacterial suspension and then removed and dabbed on filter paper before returned to the original feeder plate. The explants were co-cultivated with the agrobacterial cells under the same conditions as used in the pre-incubation phase for 40 hours.

Selection: The explants were taken from the feeder layers and put on tomato regeneration plates containing 500 mg/L Carbenicillin, and 100 mg/L Kanamycin for selection. Cotyledons explants were placed on medium with the right side upwards ensuring good contact with the nutrients and drugs. About 10 explants were placed in every plate and the plates were returned to the growth cabinet. The explants were transferred to fresh medium every 2-3 weeks. Once the regenerating material became too large for petri dishes, it was then put into larger pot (Magenta vessel).

Plant regeneration: Regenerated shoots were cut from the explants and put onto rooting medium containing 200 mg/L Carbenicillin, 100 mg/L Kanamycin. Once the shoots developed roots, they were removed the medium by washing the root gently under running water and then transferred to hydrated, autoclaved Jiffy pots (containing peat) and placed inside a sealed propagation tray to maintain humidity in short day growth room. Once roots were seen growing through the Jiffy pots, the putative transgenic plants were transferred to bigger pots containing soil and kept in the glasshouse. Confirmation of transgene: DNA was extracted from regenerated T1 tomato plants and used for PCR amplification with AtRPW8.1 and AtRPW8.2 specific primers.

Pathogen test: About 4 weeks old T1 tomato plants were inoculated with Oidium lycopersici Oxford and examined for resistance/susceptibility 10 DPI.

Transformation of barley with RPW8.1 and RPW8.2

Barley is routinely transformed by Agrobacterium tumefaciens (Tingay et al. 1997), and this method may be used as described for the production of plants transgenic for RPW8.1 and RPW8.2.

A. tumefaciens carrying RPW8.1 and RPW8.2 in a binary vector under control of a promoter constitutively expressed in barley, and with the bar gene as selectable marker on the T-DNA, is co-cultivated with immature barley embryo explants. Selection is made for bialaphos-resistant cultures, from which plants are regenerated using standard methods (Tingay et al. 1997). From more than 1,500 embryos, it is generally possible to recover more than 50 plants, more than 10 of which will grow to maturity and be fertile. Tests on their progeny for the marker gene (bar gene conferring bialaphos resistance) will identify individuals with the transgene at a single locus, which are then used to test for resistance to the powdery mildew pathogen.

Transformation of Wheat with RPW8.1 and RPW8.2

A rapid Agrobacterium tumefaciens-mediated transformation system is used for wheat (Duncan et al. 1997). This uses either freshly isolated immature embryos, precultured immature embryos, or embryogenic calli as explants. The explants are inoculated with a disarmed A. tumefaciens strain C58 (ABI) harboring the binary vector pMON18365 containing RPW8.1 and RPW8.2 under control of a promoter constitutively expressed in wheat, and a selectable marker, the neomycin phosphotransferase II gene. The inoculated immature embryos or embryogenic calli are selected on G418-containing media. Transgenic plants are regenerated from the three types of explants. The procedure is rapid, and the total time required from inoculation to the establishment of plants in soil is generally 2.5 to 3 months, with most or all transformants morphologically normal, having the insert stably integrated and segregating in a Mendelian fashion. T2 plants are tested for resistance to the wheat powdery mildew pathogen.

### Example 9

### The RPW8 Promoters

As shown in Example 8, the SE7.5 construct containing AtRPW8.1 and AtRPW8.2 under their corresponding promoters demonstrates that these AtRPW8 promoters work in tobacco (N. benthamiana and N. tobaccum).

In a separate experiment, the same construct (SE7.5) causes cell death when transiently expressed in N. bentamiana by agro-infiltration.

The RPW8 Promoter is also Wound Activated

The following were cloned into binary vector pBI101 in front of the GUS translation start by using HindIII and XbaI restriction sites: 1000 bp sequence upstream of AtRPW8.1 translation start, 1000 bp sequence upstream of AtRPW8.2 translation start, and 496 bp sequence upstream of AtHR3 translation start. All the three fusion constructs were introduced into Arabidopsis Col-0 via Agrobacterium-mediated transformation. T1 transgenic plants were selected on MS plates containing 50 mg/L Kanamycin.

Mature leaves of at least 10 T1 plants from each construct were wounded by fine forceps and then immediately immersed in GUS staining solution (50 mM Na₃PO₄, pH7.0, 1.0 mM X-Glucuronide), and incubated for ˜14 hours. Two week-old T 2 seedlings selected on MS plates containing 50 mg/L Kanamycin were treated with SA and JA (2.5 ml of 1 mM SA and 2.5 ml of 0.4 mM JA were added to the small petri dishes (4.5 cM in diameter) containing the plants) for 72 hours. Seedling were then transferred into GUS staining solution for ˜14 hours.

Initial results indicated that wounding induced GUS activity in most of the T1 transgenic plants carrying either one the 3 promoter-GUS fusion constructs. SA seemed to induce GUS activity in their T2 plants, while JA did not. These observations suggest that AtRPW8 promoters are wounding and SA responsive.

### Example 10

### Over-Expression of RPW8 Induces Cell Death

Many Arabidopsis T1 lines carrying AtRPW8.1 and AtRPW8.2 genomic sequence (either construct SE14, EE7.5 or EE6.2) showed necrotic lesions on leaves in the absence of powdery mildew pathogens. In order to further investigate whether the cell death on these plants are spontaneous, we generated T4 lines homozygous for the transgene from one T1 line, named SE14-24, which shows the most severe cell death phenotype.

Southern analysis indicated this line had a single insertion, however, that insertion might have had multiple copies of AtRPW8 tandemly linked, as it was indicated by the higher intensity of the transgene band of SE14-24 (not shown). Quantitative RT-PCR confirmed that SE14-24 T4 plants have much higher level of AtRPW8.1 and AtRPW8.2 mRNA (data not shown).

SE14-24 T4 plants growing in sterile MS medium normally do not develop necrotic lesions, but they do have spontaneous cell death when transferred to sterile soil or perlite. High light and low humidity promote cell death , while, high temperature (30° C.), high humidity and dark/low light suppress/alleviate cell death phenotype. It was also confirmed that the spontaneous cell death in the SE14-24 line starts from the palisade mesophyll cells and the cell death is associated with localised H202 accumulation.

General Methods Used (Except Where Stated Otherwise)

Pathology

O. lypcopersicum Oxford and E. orontii MGH were propagated on Lycopersicon esculentum cv Moneymaker; E. chichoracearum UCSC1 was propagated on squash, and E. cruciferarum UEA1 on oilseed rape(8). A. thaliana plants were inoculated according to Xiao et al. (1997)(8).

Molecular Biology

General Methods Were According to Sambrook et al. 1989(19)

Molecular Markers

Selected YAC from the AtEM1 contig on chromosome 3 (http://genome-www3.stanford.edu/atdb_welcome.html) and BAC ends were isolated(20), sequenced and used to develop CAPS and RFLP markers polymorphic between Ms-0 and Ler for the fine-mapping of RPW8. CAPS markers included: X1-6 from cosmid X1-11 end (primers ATCCGCCTCTTTCTTTTGGTTTTC and GTGTTACTTTTCTACAGCCAGAG; polymorphism revealed with BstNI,); B9 from cosmid B6 end (primers GTCTGAATCCGTCAAGCCTTCG and TCCATGCTTCTATATTGAAGAGC, polymorphism revealed with CfoI), and 6I2-L from BAC 6I2 end (primers GATTGTATAGGTTGGTTGATGAG and GCATCTCATTGACCTCCCTATC, polymorphism revealed with HindIII). RFLP markers included : 8E1-R from YAC 8E1 (probe amplified with primers CAGCTTCCTTCACCGTCTCATGG and CCAGGAAAATAACGGTGACGATC; polymorphism revealed with CfoI); and 3B3-L from BAC 3B3 end (probe amplified with primers GTCATCATCTAAAGAGGATAAGG and GGTTGAAAAAGTGGCTTTGGATG, polymorphism revealed with NsiI). RFLP marker Atpk41A was an EST (L05561; probe amplified with primers ATGGATCCGGCGACTAATTCACC and TGTCCTCAGGAATCTCAGAGAGC; polymorphism revealed with CfoI).

Cosmid Library

Genomic DNA from accession Ms-0 was partially digested with Sau3AI and fractions 15-25 kb were ligated into the BamHI site of vector SLJ755I5, packaged into lambda using Gigapack□ III XL Packaging Extract kit (Stratagene), and propagated in ˜60,000 colony forming units of E. coli strain DH1OB (GIBCO-BRL).

DNA Sequencing

Overlapping EcoRI and HindIII fragments of cosmids B6 and J4-2 were ligated into appropriate sites in pBluescript II SK⁺ (Stratagene), cloned in E. coli strain XL-Blue (Statagene), and sequenced. RPW8 alleles from A. thaliana accessions were amplified by PCR from genomic DNA with primers specific for RPW8.1 (GACCCGTACAGTACTAAGTCTA and GATTTCCGAAATTGATTACAAGAA) and for RPW8.2 (AACTCTTCACCTCGAGAGCTAACA and AGTCGTTTGACACAATTGGGACAT). Products from 4 independent PCRs were pooled and sequenced. DNA sequences were assembled with the Staden DNA analysis package and analysed with programmes at HGMP (hgmp.mrc.ac.uk website).

Transcript Analysis

3′RACE and 5′RACE were according to the manufacturer's instruction (GIBCO BRL). Gene specific primers for RPW8.1 were: 3′RACE: AATGGACACTAAACTTGCTGAAGT and 5′RACE: CCACAACTATTATGCTTCT, and is nested primer GAACCAAAAACGGCTCGATACTAA. Gene-specific primers for RPW8.2 were 3′RACE: GCTAAATTACGATGGGTGGTAGAT and nested primer CGATGGGTGGTAGATGTGGATGTT, and 5′RACE: GGATCGCACGGTTTGT and nested primer CTGAACTTCTTGCGTACGTTTCT. PCR products were cloned into pGEM-T easy vector (Promega) in E. coli strain XL-Blue, and sequenced.

A. thaliana Transformations

Restriction sites detected in the sequence of cosmid B6 were used to make sub-clones in vector SLJ755I5 propagated in E. coli strain DH1OB. RPW8.1 and RPW8.2 cDNAs were amplified by RT-PCR using Pfu-Turbo (Stratagene) with primers for RPW8.1 (CCGGAATTCATGCCGATTGGTGAGCTTGCGATA and CGCGGATCCTCAAGCTCTTATTTTACTACAAGC) and RPW8.2 (CCGGAATTCATGATTGCTGAGGTTGCCGCA and CCGGGATCCTCAAGAATCATCACTGCAGAACGT), and cloned into the EcoRI-BamHI site of pKMB(21) for expression under control of the constitutive viral 35 S promoter in A. thaliana Col-0. Clones were maintained in E. coli DH1OB. Agrobacterium tumefaciens strain GV3101 was transformed with plasmids by electroporation, and used for stable transformation of A. thaliana accession Col-0(10).

Misc Materials

We thank F. M. Ausubel for E. orontii MGH, M. Bardin for E. cichoracearum isolates, J. R. Botella for pKMB, S. Covey for cauliflower mosaic virus infections, S. Gurr for O. lypcopersicum Oxford, J. Jones for pSLJ755I5, J. Parker for P. parasitica Noco2, Ohio Stock Centre for BAC filters containing IGF and TAMU libraries, K. Schrick for CAPS marker g19397, M. Stammers for YAC clones and for BAC filters, and X. Dong for A. thaliana Col-0 transgenic for NahG.

### Bibliography

1. Staskawicz, B. J., Ausubel, F. M., Baker, B. J., Ellis, J. G. & Jones, J. D. (1995) Science 268, 661-7.

2. Boyes, D. C., McDowell, J. M. & Dangl, J. L. (1996) Curr Biol 6, 634-7.

3. HammondKosack, K. E. & Jones, J. D. G. (1997) Annual Review of Plant Physiology and Plant Molecular Biology 48, 575-607.

4. Pan, Q., Wendel, J. & Fluhr, R. (2000) Journal of Molecular Evolution 50, 203-213.

5. Jones, D. A. & Jones, J. D. G. (1997) Advances in Botanical Research Incorporating Advances in Plant Pathology 24, 89-167.

6. Meyers, B. C., Dickerman, A. W., Michelmore, R. W., Sivaramakrishnan, S., Sobral, B. W. & Young, N. D. (1999) Plant Journal 20, 317-332.

7. Spencer, D. M. (1978) The Powdery Mildews (Academic Press, New York).

8. Xiao, S., Ellwood, S., Findlay, K., Oliver, R. P. & Turner, J. G. (1997) Plant J 12, 757-68.

9. Ellwood, S. (1998) Characterisation of two powdery mildew diseases of Arabidopsis thaliana and positional cloning of a resistance gene (University of East Anglia, Norwich).

10. Clough, S. J. & Bent, A. F. (1998) Plant Journal 16, 735-743.

11. Saenz, G. S. & Taylor, J. W. (1999) Canadian Journal of Botany-Revue Canadienne De Botanique 77, 150-168.

12. Adam, L. & Somerville, S. C. (1996) Plant J 9, 341-56.

13. Adam, L., Ellwood, S., Wilson, I., Saenz, G., Xiao, S., Oliver, R. P., Turner, J. G. & Somerville, S. (1999) Molecular Plant-Microbe Interactions 12, 1031-1043.

14. Dixon, M. S., Hatzixanthis, K., Jones, D. A., Harrison, K. & Jones, J. D. G. (1998) Plant Cell 10, 1915-1925.

15. Dixon, M. S., Jones, D. A., Keddie, J. S., Thomas, C. M., Harrison, K. & Jones, J. D. (1996) Cell 84, 451-9.

16. Parniske, M., Hammond-Kosack, K. E., Golstein, C., Thomas, C. M., Jones, D. A., Harrison, K., Wulff, B. B. & Jones, J. D. (1997) Cell 91, 821-32.

17. Parniske, M. & Jones, J. D. G. (1999) Proceedings of the National Academy of Sciences of the United States of America 96, 5850-5855.

18. ThordalChristensen, H., Zhang, Z. G., Wei, Y. D. & Collinge, D. B. (1997) Plant Journal 11, 1187-1194.

19. Sambrook, J., Fritsch, E. F. & Maniatis, T. (1989) Molecular cloning: a laboratory manual (Cold Spring Harbour Laboratory Press, New York).

20. Schmidt, R., Cnops, G., Bancroft, I. & Dean, C. (1992) Aust. J. Plant Physiol. 19, 341-351.

21. Mylne, J. & Botella, J. R. (1998) Plant Molecular Biology 16, 257-262.

Additional References:

Brettell R, 1997. Plant Journal Agrobacterium tumefaciens-mediated barley transformation 11: 1369-1376

Duncan D R, Conner T W, Wan Y C, 1997. Plant Physiology, 115: 971-980

Filliati, J. J, et al. Bio/Technology (1987) 5:726-730

Horsch, R. B., Fry, J. E., Hoffmann, N. L., Eichholtz, D., Rogers, S. G. and I Fraley, R. T. (1985) A simple general method for transferring genes into plants. Science 227,1229-1231.

Horsch R. B. and Klee, H. J .(1986) Rapid assay of foreign gene expression in leaf discs transformed by Agrobacterium tumefaciens; Role of the T-DNA borders in the transfer process. Proc. Natl. Acad. Sci. USA 83,4428-4432.

McCormick, S. et al 1986 Plant Molecular Biology Reporter. 1987 5:380-386

McDonnell et al. 1987. Molecular Plant Biology Reporter 5:380-386.

Tingay S, McElroy D, Kalla R, Fieg S, Wang M B, Thornton S,

Sequence Listing 2. The RPW8 Locus

| mRNA RPW8.1 | complement 13878 . . . 14719 |
|---|---|
| CDS | complement(join(13990-14155;14353-14633)) |
| | /label = RPW8.1, powdery mildew resistance gene |
| | /note = “Transcription start: 14719; |
| | exon1: 14719-14353; |
| | Intron: 14352-14156; |
| | Exon2: 14155-13878; |
| | Transcription end: 13878; |
| | Protein sequence: |
| mpigelaigavlgvgagaiydrfrkardisfvhrlcatilsiepflvqidkrskvegs | |
| plrevnerltcflelayvfveaypklrrrqvlrkyryikaietielalrsiivvdfqv | |
| dqwddikeikakisemdtklaevisacskira” | |
| mRNA RPW8.2 | complement 15904 . . . 16829 |
| CDS | complement(join(16015-16243;16372-16667)) |
| | /label = RPFW8.2, powdery mildew resistance gene |
| | /note = “Transcription start: 16829; |
| | exon1: 16829-16372; |
| | Intron: 16371-16244; |
| | Exon2: 16243-15904 |
| | Transcription end: 15904; |
| | Protein sequence: |

| miaevaaggalglalsvlheavkrakdrsvttrfilhrleatidsitpivvqidkfse | |
|---|---|
| emedstsrkvnkrlklllenavslveenaelrrrnvrkkfrymrdikefeaklrwvvd | |
| vdvqvnqladikelkakmseistkldkimpqpkfeihigwcsgktnrairftfcsdds” | |
| nucleotide sequence: | |
| 13801 | catgaaacat agatctcaaa agaagcgaaa taaaaagatt attgttaatt attattttga |
| 13861 | taaaattaca catagattga gaaagagttt ttcaataatt atggqgaata agagagagag |
| 13921 | agagagaaat agatttccga aattgattac aagaagaaat aatttcaaca aagtctctgt |
| 13981 | ttttttttat caagctctta ttttactaca agcagaaata acttcagcaa gtttagtgtc |
| 14041 | catttcagat atcttggcct tgatttcttt gatatcgtcc cattgatcaa cttgaaaatc |
| 14101 | cacaactatt atgcttctta atgcaagttc tatcgtttcg attgctttga tgtacctaaa |
| 14161 | gataaacaga acaaacataa tactcgtgtt atttttccac aacatgatag gttttgtacg |
| 14221 | tttagtgttt ggagattatc gaaatcatgt aaaaaaaatt gttacaaaga agaagatatt |
| 14281 | tttctctaaa ccattaaact aagaaattag gcgatccaaa aaccaataga aattcatgtc |
| 14341 | atatatacga acctgtactt cctgagtact tgtctgcgtc tgagtttcgg ataagcctca |
| 14401 | acaaaaacat aagctaattc aaggaaacac gtgagacgtt cgttgacttc ccttaatggt |
| 14461 | gaaccttcca ctttactccg cttatcgatt tgaaccaaaa acggctcgat actaaggatt |
| 14521 | gtagcgcaga gacggtgtac gaaagatata tctcttgctt ttctgaaccg gtcgtaaatg |
| 14581 | gcttgggctc caactccaag aacagcccct atcgcaagct caccaatcgg cattttttga |
| 14641 | aagtagttgt ttagctctcg aggtgaatat agaggaatct atgtacatgg aaggatggaa |
| 14701 | ccatattaaa tagttttatg tttaacaagt taacgagtgg ttttaattat atgaagacaa |
| 14761 | ttcaagagat tgactcatag acttagtact gtacgggtca acaactctct ctttttctag |
| 14821 | gtaagaggag atcgttggat ctatatgcaa gttgtcgtga gtattaaatt acgtagaata |
| 14881 | ttattgaatt acgtcgaaga agcgagagtc aatctcactc tcaatggtta acttgtacat |
| 14941 | ttagaagaag gaaaaatcaa cgaagttggc tgagtaagaa gtgaagaaga aaaacagtga |
| 15001 | agaaagccaa aaagcagaag aggaaaatgg tggtatcaac taaaaatatt tcaacaaagg |
| 15061 | aagttactac taaaaatatt tcaacaaaag aagttactac taaaaataaa tactttgcat |
| 15121 | gttgcagtat atatttaaaa tttagaaata attatatcta ttaaaaaatc attttgtaac |
| 15181 | agatgttcga ttatgatata tagaattatt ttgtagacgt tttataaaat agtttaaaaa |
| 15241 | attatattga agatatgaga tgaaccacaa tacgtatttt tatttttcgt attttcaaat |
| 15301 | aaactcttat tattatatga aatctgaatt agcccagaat attattagat ttggtttata |
| 15361 | atttaatctc aaaattttct tccaaactga aaacagaaaa aaaaaaaaaa aaaaaaagaa |
| 15421 | gaagaagaag aagaagttaa aaaccactaa tctgaaagat ccactctaat ttgtataaat |
| 15481 | ttttcgtttt aagttcaaag atgggatcaa atcaaatgag aagaatcctt aaaaactttc |
| 15541 | atctttatgt aagaagcaaa agcaaattta gttaagcttt tttctaagtt ctttatatct |
| 15601 | tctttcagca ttaattcatt atccacaact ttgttatact cattatcctt caaacttgat |
| 15661 | tgtattgagt ttgcttctcc gttgatccta atacgctaag ttcaactctt tgtaacaact |
| 15721 | ttgttcttta aagcattttg agttctaaat aaacaaattg agagaccaat gtggcagata |
| 15781 | atcgtcattt tgagatcgtt tgttgttttt tactctacaa actttggatt cacatacata |
| 15841 | tatatatata tatatataga tatatatata tatatatatt gtaatgtaat gtatagtata |
| 15901 | tttctgaatt tctctttgtt taataaccat tggcacattt atttattttc aaagtatgtc |
| 15961 | attagattat tcatattaat acatatatat gagtcgtttg acacaattgg gacatcaaga |
| 16021 | atcatcactg cagaacgtaa atcggatcgc acggtttgtt tttcctgaac accagccgat |
| 16081 | gtggatttca aacttcggtt gaggcattat tttgtcaagt ttagtgctga tttcagacat |
| 16141 | cttggccttg agttctttga tatcagccaa ttgattaact tgaacatcca catctaccac |
| 16201 | ccatcgtaat ttagcttcga actctttgat atctctcatg tacctaaaga taaacaacac |
| 16261 | aaatataata cacatgttat tgacttaatt catagtaaat gttaggtttt gatagattta |
| 16321 | gtactgttgg gagtttatgg aaatcacata taggaactat ttagcacaaa cctgaacttc |
| 16381 | ttgcgtacgt ttctgcgtct cagctccgca ttctcctcaa caagagaaac agcgttctca |
| 16441 | aggagaagct taagacgttt attgactttc ctcgatgttg aatcttccat ttcttcactg |
| 16501 | aacttatcaa tttgaaccac caacggtgtg atactatcga ttgtagcttc gagacggtgt |
| 16561 | aagatgaatc ttgtggttac agatctatct tttgctcttt tgacggcctc gtggaggaca |
| 16621 | ctgagagcaa gtccaagagc accccctgcg gcaacctcag caatcatttt cttgaaatta |
| 16681 | gtttgttagc tctcgaggtg aagagttttt gatgagttat attgatgata ttattttgtt |
| 16741 | tggtaagaaa aatataagac catctattat attatataga ggtgaatatt tataattcct |
| 16801 | ttttcttctc aaatatttgg taaagtgttg ctctattaat tcacataatg ttagtattat |
| 16861 | acacaaatat tataagggtg aatgcaatga gaaatctatg aacatggaag tcttttgctt |
| 16921 | aacaattaag ccgtgtagtt tgtataaagt caaacggatg ttctttgttt ccgtaacttc |
| 16981 | ctacgaaaga gtgtgaataa gagatgtgtg gaccgcttgg taaagtacca tgcagttaga |
| 17041 | agcatgtacg gggtagtgaa acgtcgattt ttattataaa ataaaataat aaacgatatg |
| 17101 | tgttggaggc gtatatatat taataaatag ttaaataaca aaattaaatc gtcttttact |
| 17161 | ttttttatag ctaataaaat caaatagttt aaagtcaatt ttagatcatt gtcagtaaaa |
| 17221 | acatcattaa actcaagtct ttcaaagtta atttaattaa atttatgcag aaaattcata |
| 17281 | aaacatagat ctcaaaagaa gcaaaataaa aagattattg ttaattatta ttttgataaa |
| 17341 | attacacata gattgagaaa gagtttttca atcattattg ggaagaaggg aggaagaaaa |
| 17401 | gaaaaaacag atttctgaaa ttgattataa gaagaaataa tttcaacagt ctctgttttt |
| 17461 | ttaaatcaag ttcttatttt attacaaagt gaaataattt cagatatctt ggccttgatt |
| 17521 | tctttggtat ctttctaaaa aacaaattta gagaccaatg tggcagagaa tcgtcatttt |
| 17581 | gggatcgttt gttgtttttt actctacaaa ctttggattc acatacatat tatatgtatt |
| 17641 | gtaatgtaat gagtaatata tttctgaatg tctctttgtt tacgttacat tggcacattt |
| 17701 | atgaagacaa aagacgtttt tgattaatta tattgatgat atatataaag acaaaagacg |
| 17761 | tttcacaaaa tattaaaacc ttaggaaaga caccccattt atcatcaatg gaggtgctct |
| 17821 | tagataacaa tctagaatcc ttatcgcttt agacagctgt gttattgact agtcatcatc |
| 17881 | taaagaggat aaggattgga aacgatttga attggagacc aagtgcttgg agagtaagct |
| 17941 | tagggttgtc tttgtatgtg tgtatatata ctcctcaaga tcgatcaata acatcaagca |
| 18001 | ctttttcaac cattcttagt ctttacaatt aatgtacgaa gaggattatt atttattaaa |
| 18061 | ttacgaaaaa gaagtgaaaa tcgatctaaa tgattgactt tttacgtaga atcgtcgaat |
| 18121 | tgcatgtaca tttccaccga aattccaaaa atctgaatta caaataagtt ggaaccgatc |
| 18181 | gatcttgttt tgtatattta cgtacaaggc agacgtacat acatgtagtt tggattatca |
| 18241 | tatgtatgat caacgcaatt ttcgtgaata gaaacgtgaa tactaacaat ttcggtgaat |
| 18301 | acctaccgta aatactaaca ttaaaatcta tgacttctta aaataataat caatcaaact |
| 18361 | tttacatttg attttatatt ttcctcagtt tttaggccta tgatacacct gccttctcaa |
| 18421 | aatattagtt ccgtgatgtt tgctccatct aaggtggata tcgatc |

Sequence Listing 3: The cDNA Nucleotide Sequence of RPW8.1 from Ms-0 is Aligned with that of RPW8.1 Homologues Isolated by PCR from other A. thaliana Accessions.

| | |
|---|---|
| | 1 50 |
| RPW8.1c-Ms | ATGCCGATTG GTGAGCTTGC GATAGGGGCT GTTCTTGGAG TTGGAGCCCA |
| RPW8.1c-Wa | ATGCCGATTG GTGAGCTTGC GATAGGGGCT GTTCTTGGAG TTGGAGCCCA |
| RPW8.1c-Kas | ATGCCGATTG GTGAGCTTGC GATAGGGGCT GTTCTTGGAG TTGGAGCCCA |
| RPW8.1c-G24 | ATGCCGATTG GTGAGCTTGC GATAGGGGCT GTTCTTGGAG TTGGAGCCCA |
| RPW8.1c-Can | ATGCCGATTG GTGAGCTTGC GATAGGGGCT GTTCTTGGAG TTGGAGCCCA |
| RPW8.1c-Nd | ATGCCGATTG GTGAGCTTGC GATAGGGGCT GTTCTTGGAG TTGGAGCCCA |
| RPW8.1c-Sy ATGCCGATTG GTGAGCTTGC GATAGGGGCT GTTCTTGGAG TTGGAGCCCA | |
| RPW8.1c-Ws | ATGCCGATTG GTGAGCTTGC GATAGGGGCT GTTCTTGGAG TTGGAGCCCA |
| RPWB.1c-Ler | ATGCCGATTG GTGAGCTTGC GATAGGGGCT GTTCTTGGAG TTGGAGCCCA |
| | 51 100 |
| RPW8.1c-Ms | AGCCATTTAC GACCGGTTCA GAAAAGCAAG AGATATATCT TTCGTACACC |
| RPW8.1c-Wa | AGCCATTTAC GACCGGTTCA GAAAAGCAAG AGATATATCT TTCGTACACC |
| RPW8.1c-Kas | AGCCATTTAC GACCGGTTCA GAAAAGCAAG AGATATATCT TTCGTACACC |
| RPW8.1c-C24 | AGCCATTTAC GACCGCTTCA GAAAAGCAAG AGATATATCT TTCGTACACC |
| RPW8.1c-Can | AGCCATTTAC GACCGCTTCA GAAAAGCAAG AGATATATCT TTCGTACACC |
| RPW8.1c-Nd | AGCCATTTAC GACCGCTTCA GAAAAGCAAG AGATATATCT TTCGTACACC |
| RPW8.1c-Sy | AGCCATTTAC GACCGGTTCA GAAAAGCAAG AGATATATCT TTCGTACACC |
| RPW8.1c-Ws | AGCCATTTAC GACCGCTTCA GAAAAGCAAG AGATATATCT GTCGTAAACC |
| RPW8.1c-Ler | AGCCATTTAC GACCGCTTCA GAAAAGCAAG AGATATATCT GTCGTAAACC |
| | 101 150 |
| RPW8.1c-Ms | GTCTCTGCGC TACAATCCTT AGTATCGAGC CGTTTTTGGT TCAAATCGAT |
| RPW8.1c-Wa | GTCTCTGCGC TACAATCCTT AGTATCGAGC CGTTTTTGGT TCAAATCGAT |
| RPW8.1c-Kas | GTCTCTGCGC TACAATCCTT AGTATCGAGC CGTTTTTGGT TCAAATCGAT |
| RPW8.1c-C24 | GTCTCTGCGC TACAATCCTT AGTATCGAGC CGTTTTTGGT TCAAATCGAT |
| RPW8.1c-Can | GTCTCTGCGC TACAATCATT AGTATCGAGC CGTTTTTGGT TCAAATCGAT |
| RPW8.1c-Nd | GTCTCTGCGC TACAATCATT AGTATCGAGC CGTTTTTGGT TCAAATCGAT |
| RPW8.1c-Sy | GTCTCTGCGC TACAATCCTT AGTATCGAGC CGTTGTTGGT TCAAATCGAT |
| RPW8.1c-Ws | GTCTCTGCGC TACAATCATT AGTATCAGGC CGTTGTTGGT TCAAATCGAT |
| RPW8.1c-Ler | GTCTCTGCGC TACAATCATT AGTATCAGGC CGTTGTTGGT TCAAATCGAT |
| | 151 200 |
| RPW8.1c-Ms | AAGCGGAGTA AAGTGGAAGG TTCACCATTA AGGGAAGTCA ACGAACGTCT |
| RPW8.1c-Wa | AAGCGGAGTA AAGTGGAAGG TTCACCATTA AGGGAAGTCA ACGAACGTCT |
| RPW8.1c-Kas | AAGCGGAGTA AAGTGGAAGG TTCACCATTA AGGGAAGTCA ACGAACGTCT |
| RPW8.1c-C24 | AAGCGGAGTA AAGTGGAAGG TTCACCATTA AGGGAAGTCA ACGAACGTCT |
| RPW8.1c-Can | AAGCGGAGTA AAGTGGAAGG TTCACCATTA AGGGAAGTTA ACGAACGTCT |
| RPW8.1c-Nd | AAGCGGAGTA AAGTGGAAGG TTCACCATTA AGGGAAGTTA ACGAACGTCT |
| RPW8.1c-Sy | AAGCGGAGTA AAGTGGAAGG TTCACCATTA AGGGAAGTCA ACGAACGTCT |
| RPW8.1c-Ws | AAGCGGAGTA AAGTGGAAGG TTCACCATTA ACGGAAGTCA ACGAACGTCT |
| RPW8.1c-Ler | AAGCGGAGTA AAGTGGAAGG TTCACCATTA AGGGAAGTCA ACGAACGTCT |
| | 201 250 |
| RPW8.1c-Ms | CACGTGTTTC CTTGAATTAG CTTATGTTTT TGTTGAGGCT TATCCGAAAC |
| RPW8.1c-Wa | CACGTGTTTC CTTGAATTAG CTTATGTTTT TGTTGAGGCT TATCCGAAAC |
| RPW8.1c-Kas | CACGTGTTTC CTTGAATTAG CTTATGTTTT TGTTGAGGCT TATCCGAAAC |
| RPW8.1c-C24 | CACGTGTTTC CTTGAATTAG CTTATGTTTT TGTTGAGGCT TATCCGAAAC |
| RPW8.1c-Can | CACGTGTTTC CTTGAATTAG CTTATGTTTT AGTTGAGGCT TATCCGAAAC |
| RPW8.1c-Nd | CACGTGTTTC CTTGAATTAG CTTATGTTTT AGTTGAGGCT TATCCGAAAC |
| RPW8.1c-Sy | CACGTGTTTC CTTGAATTAG CTTATGTTTT AGTTGAGGCT TATCCGAAAC |
| RPW8.1c-Ws | CACGTGTTTC CTTGAATTAG CTTATGTTTT AGTTGAGGCT TATCCGAAAC |
| RPW8.1c-Ler | CACGTGTTTC CTTGAATTAG CTTATGTTTT TGTTGAGGCT TATCCGAAAC |
| | 251 300 |
| RPW8.1c-Ns | TCAGACGCAG ACAAGTACTC AGGAAGTACA GGTACATCAA AGCAATCGAA |
| RPW8.1c-Wa | TCAGACGCAG ACAAGTACTC AGGAAGTACA GGTACATCAA AGCAATCGAA |
| RPW8.1c-Kas | TCAGACGCAG ACAAGTACTC AGGAAGTACA GGTACATCAA AGCAATCGAA |
| RPW8.1c-C24 | TCAGACGCAG ACAAGTACTC AGGAAGTACA GGTACATCAA AGCAATCGAA |
| RPW8.1c-Can | TCAGACGCAG ACAAGTACTC AGGAAGTACA GGTGCATCAA AGCAATCGAA |
| RPW8.1c-Nd | TCAGACGCAG ACAAGTACTC AGGAAGTACA GGTGCATCAA AGCAATCGAA |
| RPW8.1c-Sy | TCAGACGCAG ACAAGTACTC AGGAAGTACA GGTACATCAA AGCAATCGAA |
| RPW8.1c-Ws | TCAGACGCAG ACAAGTACTC AGGAAGTACA GGTACATCAA AGCAATCGAA |
| RPW8.1c-Ler | TCAGAGGCAG ACAAGTACTC AGGAAGTACA GGTACATCAA AGCAATCGAA |
| | 301 350 |
| RPW8.1c-Ms | ACGATAGAAC TTGCATTAAG AAGCATAATA GTTGTGGATT TTCAAGTTGA |
| RPW8.1c-Wa | ACGATAGAAC TTGCATTAAG AAGCATAATA GTTGTGGATT TTCAAGTTGA |
| RPW8.1c-Kas | ACGATAGAAC TTGCATTAAG AAGCATAATA GTTGTGGATT TTCAAGTTGA |
| RPW8.1c-C24 | ACGATAGAAC TTGCATTAAG AAGCATAATA GTTGTGGATT TTCAAGTTGA |
| RPW8.1c-Can | ACGATAGAAC TTGCATTAAG AAGGATAATA GTTGTGGATT TTCAAGTTGA |
| RPW8.1c-Nd | ACGATAGAAC TTGCATTAAG AAGGATAATA GTTGTGGATT TTCAAGTTGA |
| RPW8.1c-Sy | ACGATAGAAC TTGCATTAAG AAGCATAATA GTTGTGGATT TTCAAGTTGA |
| RPW8.1c-Ws | ACGATAGAAC TTGCATTAAG AAGCATAATA GTTGTGGATT TTCAAGTTGA |
| RPW8.1c-Ler | ACGATAGAAC TTGCATTAAG AAGCATAATA GTTGTGGATT TTCAAGTTGA |
| | 351 400 |
| RPW8.1c-Ms | TCAATGGGAC GAT....... .......... .......... .......... |
| RPW8.1c-Wa | TCAATGGGAC GAT....... .......... .......... .......... |
| RPW8.1c-Kas | TCAATGGGAC GAT....... .......... .......... .......... |
| RPW8.1c-C24 | TCAATGGGAC GAT....... .......... .......... .......... |
| RPW8.1c-Can | TCAATGGGAC GATATCAAAG AAATCAAGGC CAAGATATCT GAAACGGACA |
| RPW8.1c-Nd | TCAATGGGAC GATATCAAAG AAATCAAGGC CAAGATATCT GAAACGGACA |
| RPW8.1c-Sy | TCAATGGGAC GAT....... .......... .......... .......... |
| RPW8.1c-Ws | TCAATGGGAC GAT....... .......... .......... .......... |
| RPW8.1c-Ler TCAATGGGAC GAT....... .......... .......... .......... | |
| | 401 450 |
| RPW8.1c-Ms | .......... .......... ......ATCA AAGAAATCAA GGCCAAGATA |
| RPW8.1c-Wa | .......... .......... ......ATCA AAGAAATCAA GGCCAAGATA |
| RPW8.1c-Kas | .......... .......... ......ATCA AAGAAATCAA GGCCAAGATA |
| RPW8.1c-C24 | .......... .......... ......ATCA AAGAAATCAA GGCCAAGATA |
| RPW8.1c-Can | CTAAACTTGC TGATCAATGG GACGATATCA AAGAAATCAA GGCCAAGATA |
| RPW8.1c-Nd | CTAAAGTTGC TGATCAATGG GACGATATCA AAGAAATCAA GGCCAAGATA |
| RPW8.1c-Sy | .......... .......... ......ATCA AAGAAATCAA GGCCAAGATA |
| RPW8.1c-Ws | .......... .......... ......ATCA AAGAAATCAA GGCCAAGATA |
| RPW8.1c-Ler | .......... .......... ......ATCA AAGAAATCAA GGCCAAGATA |
| | 451 500 |
| RPW8.1c-Ms | TCTGAAATGG ACACTAAACT TGCTGAAGTT ATTTCTGCTT GTAGTAAAAT |
| RPW8.1c-Wa | TCTGAAATGG ACACTAAACT TGCTGAAGTT ATTTCTGCTT GTAGTAAAAT |
| RPW8.1c-Kas | TCTGAAATGG ACACTAAACT TGCTGAAGTT ATTTCTGCTT GTAGTAAAAT |
| RPW8.1c-C24 | TCTGAAATGG ACACTAAACT TGCTGAAGTT ATTTCTGCTT GTAGTAAAAT |
| RPW8.1c-Can | TCTGAAATGG ACACTAAACT TGCTGAAGTT ATTTCTGCTT GTAGTAAAAT |
| RPW8.1c-Nd | TCTGAAATGG ACACTAAACT TGCTGAAGTT ATTTCTGCTT GTAGTAAAAT |
| RPW8.1c-Sy | TCTGAAATGG ACACTAAACT TGCTGAAGTT ATTTCTGCTT GTAGTAAAAT |
| RPW8.1c-Ws | TCTGAAATGG ACACTAAACT TGCTGAAGTT ATTTCTGCTT GTAGTAAAAT |
| RPW8.1c-Ler | TCTGAAATGG AGACTAAACT TGCTGAAGTT ATTTCTGCTT GTAGTAAAAT |
| | 501 |
| RPW8.1c-Ms | AAGAGCTTGA |
| RPW8.1c-Wa | AAGAGCTTGA |
| RPW8.1c-Kas | AAGAGCTTGA |
| RPW8.1c-C24 | AAGAGCTTGA |
| RPW8.1c-Can | AAGAACTTGA |
| RPW8.1c-Nd | AAGAACTTGA |
| RPW8.1c-Sy | AAGAGCTTGA |
| RPW8.1c-Ws | AAGAGCTTGA |
| RPW8.1c-Ler | AAGAACTTGA |
| | 1 50 |
| RPW8.1p-Ms | MPIGELAIGA VLGVGAQAIY DRFRKARDIS FVHRLCATIL SIEPFLVQID |
| RPW8.1p-Wa | ---------- ---------- ---------- ---------- ---------- |
| RPW8.1p-Kas | ---------- ---------- ---------- ---------- ---------- |
| RPW8.1p-C24 | ---------- ---------- ---------- ---------- ---------- |
| RPW8.1p-Can | ---------- ---------- ---------- ---------I ---------- |
| RPW8.1p-Nd | ---------- ---------- ---------- ---------I ---------- |
| RPW8.1p-Sy | ---------- ---------- ---------- ---------- ----L----- |
| RPW8.1p-Ws | ---------- ---------- ---------- V-N------I --R-L----- |
| RPW8.1p-Ler | ---------- ---------- ---------- V-N------I --R-L----- |
| | 51 100 |
| RPW8.1p-Ms | KRSKVEGSPL REVNERLTCF LELAYVFVEA YPKLRRRQVL RKYRYIKAIE |
| RPW8.1p-Wa | ---------- ---------- ---------- ---------- ---------- |
| RPW8.1p-Kas | ---------- ---------- ---------- ---------- ---------- |
| RPW8.1p-C24 | ---------- ---------- ---------- ---------- ---------- |
| RPW8.1p-Can | ---------- ---------- ------L--- ---------- ----C----- |
| RPW8.1p-Nd | ---------- ---------- ------L--- ---------- ----C----- |
| RPW8.1p-Sy | ---------- ---------- ------L--- ---------- ---------- |
| RPW8.1p-Ws | ---------- ---------- ------L--- ---------- ---------- |
| RPW8.1p-Ler | ---------- ---------- ---------- ---------- ---------- |
| | 101 150 |
| RPW8.1p-Ms | TIELALRSII VVDFQVDQWD .......... .......... .DIKEIKAKI |
| RPW8.1p-Wa | ---------- ---------- .......... .......... .--------- |
| RPW8.1p-Kas | ---------- ---------- .......... .......... .--------- |
| RPW8.1p-C24 | ---------- ---------- .......... .......... .--------- |
| RPW8.1p-Can | -------R-- ---------- DIKEIKAKIS ETDTKLADQW D--------- |
| RPW8.1p-Nd | -------R-- ---------- DIKEIKAKIS ETDTKLADQW D--------- |
| RPW8.1p-Sy | ---------- ---------- .......... .......... .--------- |
| RPW8.1p-Ws | ---------- ---------- .......... .......... .--------- |
| RPW8.1p-Ler | ---------- ---------- .......... .......... .--------- |
| | 151 169 |
| RPW8.1p-Ms | SEMDTKLAEV ISACSKIRA |
| RPW8.1p-Wa | ---------- --------- |
| RPW8.1p-Kas | ---------- --------- |
| RPW8.1p-C24 | ---------- --------- |
| RPW8.1p-Can | ---------- --------T |
| RPW8.1p-Nd | ---------- --------T |
| RPW8.1p-Sy | ---------- --------- |
| RPW8 1p-Ws | ---------- --------- |
| RPW8.1p-Ler | ---------- --------T |

Sequence Listing 4: The Predicted Amino Acid Sequence of RPW8.1 from Ms-0 is Aligned with RPW8.1 Homologues Isolated by PCR from other A. thaliana Accessions

| | |
|---|---|
| | 1 50 |
| RPW8.1p-Ms | MPIGELAIGA VLGVGAQAIY DRFRKARDIS FVHRLCATIL SIEPFLVQID |
| RPW8.1p-Wa | ---------- ---------- ---------- ---------- ---------- |
| RPW8.1p-Kas | ---------- ---------- ---------- ---------- ---------- |
| RPW8.1p-C24 | ---------- ---------- ---------- ---------- ---------- |
| RPW8.1p-Can | ---------- ---------- ---------- ---------I ---------- |
| RPW8.1p-Nd | ---------- ---------- ---------- ---------I ---------- |
| RPW8.1p-Sy | ---------- ---------- ---------- ---------- ----L----- |
| RPW8.1p-Ws | ---------- ---------- ---------- V-N------I --R-L----- |
| RPW8.1p-Ler | ---------- ---------- ---------- V-N------I --R-L----- |
| | 51 100 |
| RPW8.1p-Ms | KRSKVEGSPL REVNERLTCF LELAYVFVEA YPKLRRRQVL RKYRYIKAIE |
| RPW8.1p-Wa | ---------- ---------- ---------- ---------- ---------- |
| RPW8.1p-Kas | ---------- ---------- ---------- ---------- ---------- |
| RPW8.1p-C24 | ---------- ---------- ---------- ---------- ---------- |
| RPW8.1p-Can | ---------- ---------- ------L--- ---------- ----C----- |
| RPW8.1p-Nd | ---------- ---------- ------L--- ---------- ----C----- |
| RPW8.1p-Sy | ---------- ---------- ------L--- ---------- ---------- |
| RPW8.1p-Ws | ---------- ---------- ------L--- ---------- ---------- |
| RPW8.1p-Ler | ---------- ---------- ------L--- ---------- ---------- |
| | 101 150 |
| RPW8.1p-Ms | TIELALRSII VVDFQVDQWD .......... .......... .DIKEIKAKI |
| RPW8.1p-Wa | ---------- ---------- .......... .......... .--------- |
| RPW8.1p-Kas | ---------- ---------- .......... .......... .--------- |
| RPW8.1p-C24 | ---------- ---------- .......... .......... .--------- |
| RPW8.1p-Can | -------R-- ---------- DIKEIKAKIS ETDTKLADQW D--------- |
| RPW8.1p-Nd | -------R-- ---------- DIKEIKAKIS ETDTKLADQW D--------- |
| RPW8.1p-Sy | ---------- ---------- .......... .......... .--------- |
| RPW8.1p-Ws | ---------- ---------- .......... .......... .--------- |
| RPW8.1p-Ler | ---------- ---------- .......... .......... .--------- |
| | 151 169 |
| RPW8.1p-Ms | SEMDTKLAEV ISACSKIRA |
| RPW8.1p-Wa | ---------- --------- |
| RPW8.1p-Kas | ---------- --------- |
| RPW8.1p-C24 | ---------- --------- |
| RPW8.1p-Can | ---------- --------T |
| RPW8.1p-Nd | ---------- --------T |
| RPW8.1p-Sy | ---------- --------- |
| RPW8.1p-Ws | ---------- --------- |
| RPW8.1p-Ler | ---------- --------T |

Sequence Listing 5: The cDNA Nucleotide Sequence of RPW8.2 from Ms-0 is Aligned with that of RPW8.2 Homologues Isolated by PCR from other A. thaliana Accessions.

| | |
|---|---|
| | 1 50 |
| RPW8.2c-Ms | ATGATTGCTG AGGTTGCCGC AGGGGGTGCT CTTGGACTTG CTCTCAGTGT |
| RPW8.2c-Wa | ATGATTGCTG AGGTTGCCGC AGGGGGTGCT CTTGGACTTG CTCTCAGTGT |
| RPW8.2c-Kas | ATGATTGCTG AGGTTGCCGC AGGGGGTGCT CTTGGACTTG CTCTCAGTGT |
| RPW8.2c-C24 | ATGATTGCTG AGGTTGCGGC AGGGGGTGCT CTTGGACTTG CTCTCAGTGT |
| RPW8.2c-Can | ATGATTGCTG AGGTTGCCGC AGGGGGTGCT CTTGGACTTG CTCTCAGTGT |
| RPW8.2c-Nd | ATGATTGCTG AGGTTGCGGC AGGGGGTGCT CTTGGACTTG GTCTCAGTGT |
| RPW8.2c-Sy | ATGATTGCTG AGGTTGCCGC AGGGGGTGGT CTTGGACTTG CTCTCAGTTT |
| RPW8.2c-Ws | ATGATTGCTG AGGTTGCGGC AGGGGGTGCT CTTGGACTTG CTCTCAGTTT |
| RPW8.2c-Ler | ATGATTGCTG AGGTTGCGGC AGGGGGTGCT CTTGGACTTG CTCTCAGTGT |
| | 51 100 |
| RPW8.2c-Ms | CCTCCACGAG GCCGTCAAAA GAGCAAAAGA TAGATCTGTA ACCACAAGAT |
| RPW8.2c-Wa | CCTCCACGAG GCCGTCAAAA GAGCAAAAGA TAGATCTGTA ACCACAAGAT |
| RPW8.2c-Kas | CCTCCACGAG GCCGTCAAAA GAGCAAAAGA TAGATCTGTA ACCACAAGAT |
| RPW8.2c-C24 | CCTTCAAGAG GCCGTCAAAA GAGCAAAAGA TAGATCTGTA ACCACAAGAT |
| RPW8.2c-Can | CCTCCACGAG GCCGTCAAAA GAGCAAAAGA TAGATCTGTA ACCACAAGAT |
| RPW8.2c-Nd | CCTCCACGAG GCCGTCAAAA GAGCAAAAGA TAGATCTGTA ACCACAAGAT |
| RPW8.2c-Sy | CCTCCACGAG GCCGTCAAAA GAGCAAAAGA TAGATCTGTA ACCACAAGAT |
| RPW8.2c-Ws | CCTCCACGAG GCCGTCAAAA GAGCAAAAGA TAGATCTGTA ACCACAAGAT |
| RPW8.2c-Ler | CCTCCACGAG GCCGTCAAAA GAGCAAAAGA TAGATCTGTA ACCACAAGAT |
| | 101 150 |
| RPW8.2c-Ms | TCATCTTACA CCGTCTCGAA GCTACAATCG ATAGTATCAC ACCGTTGGTG |
| RPW8.2c-Wa | TCATCTTACA CCGTCTCGAA GCTACAATCG ATAGTATCAC ACCGTTGGTG |
| RPW8.2c-Kas | TCATCTTACA CCGTCTCGAA GCTACAATCG ATAGTATCAC ACCGTTGGTG |
| RPW8.2c-C24 | TCATCTTACA CCGTCTCGAA GCTACAATCG ATAGTATCAC TCCGTTGGTG |
| RPW8.2c-Can | TCATCTTACA CCGTCTCGAA GCTACAATCG ATAGTATCAC ACCGTTGGTG |
| RPW8.2c-Nd | TCATCTTACA CCGTCTCGAA GCTACAATCG ATAGTATCAC TCCGTTGGTG |
| RPW8.2c-Sy | TCATCTTACA CCGTCTCGAA GCTACAATCG ATAGTATCAC TCCGTTGGTG |
| RPWB.2c-Ws | TCATCTTACA CCGTCTCGAA GCTACAATCG ATAGTATCAC TCCGTTGGTG |
| RPW8.2c-Ler | TCATCTTACA CCGTCTCGAA GCTACAATCG ATAGTATCAC TCCGTTGGTG |
| | 151 200 |
| RPW8.2c-Ms | GTTCAAATTG ATAAGTTCAG TGAAGAAATG GAAGATTCAA CATCGAGGAA |
| RPW8.2c-Wa | GTTCAAATTG ATAAGTTCAG TGAAGAAATG GAAGATTCAA CATCGAGGAA |
| RPW8.2c-Kas | GTTCAAATTG ATAAGTTCAG TGAAGAAATG GAAGATTCAA CATCGAGGAA |
| RPW8.2c-C24 | GTTCAAATTG ATAAGTTCAG TGAAGAAATG GAAGATTCAT CATCGAGGAA |
| RPW8.2c-Can | GTTCAAATTG ATAAGTTCAG TGAAGAAATG GAAGATTCAT CATCGAGGAA |
| RPW8.2c-Nd | GTTCAAATTG ATAAGTTCAG TGAAGAAATG GAAGATTCAT CATCGAGGAA |
| RPW8.2c-Sy | GTTCAAATTG ATAAGTTCAG TGAAGAAATG GAAGATTCAT GATCGAGGAA |
| RPWS.2c-Ws | GTTCAAATTG ATAAGTTCAG TGAAGAAATG GAAGATTCAT CATCGAGGAA |
| RPW8.2c-Ler | GTTCAAATTG ATAAGTTCAG TGAAGAAATG GAAGATTCAT CATCGAGGAA |
| | 201 250 |
| RPW8.2c-Ms | AGTCAATAAA CGTCTTAAGC TTCTCCTTGA GAACGCTGTT TCTCTTGTTG |
| RPWB.2c-Wa | AGTCAATAAA CGTCTTAAGC TTCTCCTTGA GAACGCTGTT TCTCTTGTTG |
| RPWB.2c-Kas | AGTCAATAAA CGTCTTAAGC TTCTCCTTGA GAACGCTGTT TCTCTTGTTG |
| RPW8.2c-C24 | AGTCAATAAA CGTCTTAAGC TTCTCCTTGA GAAGGCTGTT TCTCTTGTTG |
| RPW8.2c-Can | AGTCAATAAA CGTCTTAAGC TTCTCCTTGA GAACGCTGTT TCTCTTGTTG |
| RPW8.2c-Nd | AGTCAATAAA CGTCTTAAGC TTCTCCTTGA GAAGGCTGTT TCTCTTGTTG |
| RPWB.2c-Sy | AGTCAATAAA CGTCTTAAGC TTCTCCTTGA GAACGCTGTT TGTCTTGTTG |
| RPW8.2c-Ws | AGTCAATAAA CGTCTTAAGC TTCTCCTTGA GAACGCTGTT TCTCTTGTTG |
| RPW8.2c-Ler | AGTCAATGAA CGTCTTAAGC TTCTCCTTGA GAACGCTGTT TCTCTTGTTG |
| | 251 300 |
| RPW8.2c-Ms | AGGAGAATGC GGAGCTGAGA CGCAGAAACG TACGCAAGAA GTTCAGGTAC |
| RPW8.2c-Wa | AGGAGAATGC GGAGCTGAGA CGCAGAAACG TACGCAAGAA GTTCAGGTAC |
| RPW8.2c-Kas | AGGAGAATGC GGAGCTGAGA CGCAGAAACG TACGCAAGAA GTTCAGGTAC |
| RPW8.2c-C24 | AGGAGAATGC GGAGCTGAGA CGCAGAAACG TACGCAAGAA GTTCAGGTAC |
| RPW8.2c-Can | AGGAGAATGC GGAGCTGAGA CGCAGAAACG TACGCAAGAA GTTCAGGTAC |
| RPW8.2c-Nd | AGGAGAATGC GGAGCTGAGA CGCAGAAACG TAGGCAAGAA GTTCAGGTAC |
| RPW8.2c-Sy | AGGAGAATGC GGAGCTGAGA CGCAGAAACG TACGCAAGAA GTTCAGGTAC |
| RPW8.2c-Ws | AGGAGAATGC GGAGCTGAGA CGCAGAAACG TACGCAAGAA GTTCAGGTAC |
| RPW8.2c-Ler | AGGAGAATGC GGAGCTGAGA CGCAGAAACG TACGCAAGAA GTTCAGGTAC |
| | 301 350 |
| RPW8.2c-Ms | ATGAGAGATA TCAAAGAGTT CGAAGCTAAA TTACGATGGG TGGTAGATGT |
| RPW8.2c-Wa | ATGAGAGATA TCAAAGAGTT CGAAGCTAAA TTACGATGGG TGGTAGATGT |
| RPW8.2c-Kas | ATGAGAGATA TCAAAGAGTT CGAAGCTAAA TTACGATGGG TGGTAGATGT |
| RPW8.2c-C24 | ATGAGAGATA TCAAAGAGTT CGAAGCTAAG ATACGATGGG TGGTAGGTGT |
| RPW8.2c-Can | ATGAGAGATA TCAAAGAGTT CGAAGCTAAA TTACGATGGG TGGTAGGTGT |
| RPW8.2c-Nd | ATGAGAGATA TCAAAGAGTT CGAAGCTAAA TTACGATGGG TGGTAGGTGT |
| RPW8 2c-Sy | ATGAGAGATA TCAAAGAGTT CGAAGCTAAA TTACGATGGG TGGTAGGTGT |
| RPW8.2c-Ws | ATGAGAGATA TCAAAGAGTT CGAAGCTAAA TTACGATGGG TGGTAGGTGT |
| RPW8.2c-Ler | ATGAGAGATA TCAAAGAGTT GGAAGCTAAA TTACGATGGG TGGTAGGTGT |
| | 351 400 |
| RPW8.2c-Ms | GGATGTTCAA GTTAATCAAT TGGCTGATAT CAAAGAACTC AAGGCCAAGA |
| RPW8.2c-Wa | GGATGTTCAA GTTAATCAAT TGGCTGATAT CAAAGAACTC AAGGCCAAGA |
| RPW8.2c-Kas | GGATGTTCAA GTTAATCAAT TGGCTGATAT CAAAGAACTC AAGGCCAAGA |
| RPW8.2c-C24 | GGATGTTCAA GTTAATCAAT TGGCTGATAT CAAAGAACTC AAGGCCAAGA |
| RPW8.2c-Can | GGATGTTCAA GTTAATCAAT TGGCTGATAT CAAAGAACTC AAGGCCAAGA |
| RPW8.2c-Nd | GGATGTTCAA GTTAATCAAT TGGCTGATAT CAAAGAACTC AAGGCCAAGA |
| RPW8.2c-Sy | GGATGTTCAA GTTAATCAAT TGGCTGATAT CAAAGAACTC AAGGCCAAGA |
| RPW8.2c-Ws | GGATGTTCAA GTTAATCAAT TGGCTGATAT CAAAGAACTC AAGGCCAAGA |
| RPW8.2c-Ler | GGATGTTCAA GTTAATCAAT TGGCTGATAT CAAAGAACTC AAGGCCAAGA |
| | 401 450 |
| RPW8.2c-Ms | TGTCTGAAAT CAGCACTAAA CTTGACAAAA TAATGCCTCA ACCGAAGTTT |
| RPW8.2c-Wa | TGTCTGAAAT CAGCACTAAA CTTGACAAAA TAATGCCTCA ACCGAAGTTT |
| RPW8.2c-Kas | TGTCTGAAAT CAGCACTAAA CTTGACAAAA TAATGCCTCA ACCGAAGTTT |
| RPW8.2c-C24 | TGTCTGAAAT CAGCACTAAA CTTGACAAAA TAATGCCTCA ACCGAAGTTT |
| RPW8.2c-Can | TGTCTGAAAT CAGCACTAAA CTTGACAAAA TAATGCCTCA ACCGAAGTTT |
| RPW8.2c-Nd | TGTCTGAAAT CAGCACTAAA CTTGACAAAA TAATGCCTCA AGCGAAGTTT |
| RPW8.2c-Sy | TGTCTGAAAT CAGCACTAAA CTTGACAAA. TAATGCCTCA ACCGAAGTTT |
| RPW8.2c-Ws | TGTCTGAAAT CAGCACTAAA CTTGACAAA. TAATGCCTCA ACCGAAGTTT |
| RPW8.2c-Ler | TGTCTGAAAT CAGCACTAAA CTTGACAAAA TAATGCCTCA ACCGAAGTTT |
| | 451 500 |
| RPW8.2c-Ms | GAAATCCACA TCGGCTGGTG TTCAGGAAAA ACAAACCGTG CGATCCGATT |
| RPW8.2c-Wa | GAAATCCACA TCGGCTGGTG TTCAGGAAAA ACAAACCGTG CGATCCGATT |
| RPW8.2c-Kas | GAAATCCACA TCGGCTGGTG TTCAGGAAAA ACAAACCGTG CGATCCGATT |
| RPW8.2c-C24 | GAAATCCACA TCGGCTGGTG TTCAGGAAAA AAAAACCGTG CGATCCGATT |
| RPW8.2c-Can | GAAATCCACA TCGGCTGGTG TTCAGGAAAA ACAAACCGTG CGATCCGATT |
| RPW8.2c-Nd | GAAATCCACA TCGGCTGGTG TTCAGGAAAA AAAAAGCGTG CGATCCGATT |
| RPW8.2c-Sy | GAAATCCACA TCGGCTGGTG TTCAGGAAAA AAAAACCGTG CGATCCGATT |
| RPW8.2c-Ws | GAAATCCACA TCGGCTGGTG TTCAGGAAAA AAAAACCGTG CGATCCGATT |
| RPW8.2c-Ler | GAAATCCACA TCGGCTGGTG TTCAGGAAAA ACAAACCGTG CGATCCGATT |
| | 501 525 |
| RPW8.2c-Ms | TACGTTCTGC AGTGATGATT CTTGA |
| RPW8.2c-Wa | TACGTTCTGC AGTGATGATT CTTGA |
| RPW8.2c-Kas | TACGTTCTGC AGTGATGATT CTTGA |
| RPW8.2c-C24 | TACGTTCTGC AGTGATGATT CTTGA |
| RPW8.2c-Can | TACGTTCTGC AGTGATGATT CTTGA |
| RPW8.2c-Nd | TACGTTCTGC AGTGATGATT CTTGA |
| RPW8.2c-Sy | TACGTTCTGC AGTGATGATT CTTGA |
| RPW8.2c-Ws | TACGTTCTGC AGTGATGATT CTTGA |
| RPW8.2c-Ler | TACGTTCTGC AGTGATGATT CTTGA |

Sequence Listing 6: The Predicted Amino Acid Sequence of RPW8.2 from Ms-0 is Aligned with RPW8.2 Homologues Isolated by PCR from other A. thaliana Accessions

| | |
|---|---|
| | 1 50 |
| RPWB.2p-Ms | MIAEVAAGGA LGLALSVLHE AVKRAKDRSV TTRFILHRLE ATIDSITPLV |
| RPW8.2p-Wa | ---------- ---------- ---------- ---------- ---------- |
| RPW8.2p-Kas | ---------- ---------- ---------- ---------- ---------- |
| RPW8.2p-C24 | ---------- --------Q- ---------- ---------- ---------- |
| RPW8.2p-Can | ---------- ---------- ---------- ---------- ---------- |
| RPW8.2p-Nd | ---------- ---------- ---------- ---------- ---------- |
| RPW8.2p-Sy | ---------- ------F--- ---------- ---------- ---------- |
| RPW8.2p-Ws | ---------- ------F--- ---------- ---------- ---------- |
| RPW8.2p-Ler | ---------- ---------- ---------- ---------- ---------- |
| | 51 100 |
| RPW8.2p-Ms | VQIDKFSEEM EDSTSRKVNK RLKLLLENAV SLVEENAELR RRNVRKKFRY |
| RPW8.2p-Wa | ---------- ---------- ---------- ---------- ---------- |
| RPW8.2p-Kas | ---------- ---------- ---------- ---------- ---------- |
| RPW8.2p-C24 | ---------- ---S------ ---------- ---------- ---------- |
| RPW8.2p-Can | ---------- ---S------ ---------- ---------- ---------- |
| RPW8.2p-Nd | ---------- ---S------ ---------- ---------- ---------- |
| RPW8.2p-Sy | ---------- ---S------ ---------- ---------- ---------- |
| RPW8.2p-Ws | ---------- ---S-----E ---------- ---------- ---------- |
| RPW8.2p-Ler | ---------- ---S-----E ---------- ---------- ---------- |
| | 101 150 |
| RPW8.2p-Ms | MRDIKEFEAK LRWVVDVDVQ VNQLADIKEL KAKMSEISTK LDKIMPQPKF |
| RPW8.2p-Wa | ---------- ---------- ---------- ---------- ---------- |
| RPW8.2p-Kas | ---------- ---------- ---------- ---------- ---------- |
| RPW8.2p-C24 | ---------- I----G---- ---------- ---------- ---------- |
| RPW8.2p-Can | ---------- -----G---- ---------- ---------- ---------- |
| RPW8.2p-Nd | ---------- -----G---- ---------- ---------- ---------- |
| RPW8.2p-Sy | ---------- -----G---- ---------- ---------- ---------- |
| RPW8.2p-Ws | ---------- -----G---- ---------- ---------- ---------- |
| RPW8.2p-Ler | ---------- -----G---- ---------- ---------- ---------- |
| | 151 174 |
| RPW8.2p-Ms | EIHIGWCSGK TNRAIRFTFC SDDS |
| RPW8.2p-Wa | ---------- ---------- ---- |
| RPW8.2p-Kas | ---------- ---------- ---- |
| RPW8.2p-C24 | ---------- K--------- ---- |
| RPW8.2p-Can | ---------- ---------- ---- |
| RPW8.2p-Nd | ---------- K--------- ---- |
| RPW8.2p-Sy | .......... .......... .... |
| RPW8.2p-Ws | .......... .......... .... |
| RPW8.2p-Le | ---------- ---------- ---- |

Sequence Listing 7: BrHR1 Genomic Sequence (756 bp)

Atgcctattggtgaagttattgtaggggctgctcttggaattactctgcaagtgcttcatgaagctatcataaa
agcaaaagatagatcttcaaccaaaaaaagtatcttggaccgcctcgatgctacaatctccaggatcactccgt
tggtggttcatgtcgataagatcagcaaaagagtagaagattctgagaggaaagtcattgaagaactcaagcgt
cttcttgaaaaggctgtttctcttgttgaggcttatgcagaactcagacgcagaaacctacacaagaagcatag
gtttgtatagtttatataatacatgaaatacttgaaaaagtctttgtgatttcttaaaatgtttttatttggtt
tacataatatttatgtgttgttgatatataggtgcaagagtagaatcaaagagttagaagtttcattaagatgg
atgatagatgtggatgttcaagtcaaccaatggctagatatcaaaaaactcgtggttaagatgtctgaaatgaa
cacaaaactcgacaagatcacgtgccaaccaactgatggtagttgtttcaagagcaatgatagcacatcaccag
tgttttcacaaagtagtagtagtctcgaagcaacagacggatcttcagaggaagatgaagaagaaagcccaagt
aatggatctgaaccaaggatcgatatccacctgcgatggagttcaagaaaaggaagaaaagatcgtgagatccg
attcatggccaagtga

Sequence Listing 8: Predicted BrHR1 cDNA Sequence (651 bp)

Atgcctattggtgaagttattgtaggggctgctcttggaattactctgcaagtgcttcatgaagctatcataaa
agcaaaagatagatcttcaaccaaaaaaagtatcttggaccgcctcgatgctacaatctccaggatcactccgt
tggtggttcatgtcgataagatcagcaaaagagtagaagattctgagaggaaagtcattgaagaactcaagcgt
cttcttgaaaaggctgtttctcttgttgaggcttatgcagaactcagacgcagaaacctacacaagaagcatag
gtgcaagagtagaatcaaagagttagaagtttcattaagatggatgatagatgtggatgttcaagtcaaccaat
ggctagatatcaaaaaactcgtggttaagatgtctgaaatgaacacaaaactcgacaagatcacgtgccaacca
actgatggtagttgtttcaagagcaatgatagcacatcaccagtgttttcacaaagtagtagtagtctcgaagc
aacagacggatcttcagaggaagatgaagaagaaagcccaagtaatggatctgaaccaaggatcgatatccacc
tgcgatggagttcaagaaaaggaagaaaagatcgtgagatccgattcatggccaagtga

Sequence Listing 9: Predicted BrHR1 Protein Sequence (217 aa)

Mpigevivgaalgitlqvlheaiikakdrsstkksildrldatisritplvvhvdkiskrvedserkvieelkr
llekavslveayaelrrrnlhkkhrcksrikelevslrwmidvdvqvnqwldikklvvkmsemntkldkitcqp
tdgscfksndstspvfsqssssleatdgsseedeeespsngsepridihlrwssrkgrkdreirfmak

Sequence Listing 10: BrHR2 Genomic Sequence (753 bp)

Atgcctattggtgaggttattgtaggggctgctcttggaattactctgcaagtgcttcatcaagctatcataaa
agcaaaagatagatcttcaaccacaaaatgtatcttggtccgcctcgatgctacaatctccaggatcactccgt
tggtggttcatgtcgataagatcagcaaaagagtagaagattctgagaggaaagtcattgaagaactcaagcgt
cttcttgaaaaggctgtttctcttgttgaggcttatgcagaactcagacgcagaaacctacacaagaagcattg
gtttgtatagtttatataatacatgaaatacttgaaaaagtctttgtgatttcttaaaatgtttttatttggtt
tacataatatttatgtgttgttgatatataggtacaagagtagaatcaaagagttagaagcttcattaagatgg
atggtagatgtggatgttcaagtcaaccaatggctagatatcaaagaactcgtggctaagatgtctgaaatgaa
cacaaaactcgacaagatcacgagccaaccaactgatggtagttgtttcaagagcaatgatagcatatcaccag
tgttatcacaaagtagtaggatcgaagcaacagacggatcttcagaggaagatgaagaagaaagctcaagtaat
ggatccgaaccaaggatcgatatccacctgcgatggagttcaagaaaaggaagaaaagatcgtgagatccgatt
cacggccaagtga

Sequence Listing 11: Predicted BrHR2 cDNA Sequence (648 bp)

Atgcctattggtgaggttattgtaggggctgctcttggaattactctgcaagtgcttcatcaagctatcataaa
agcaaaagatagatcttcaaccacaaaatgtatcttggtccgcctcgatgctacaatctccaggatcactccgt
tggtggttcatgtcgataagatcagcaaaagagtagaagattctgagaggaaagtcattgaagaactcaagcgt
cttcttgaaaaggctgtttctcttgttgaggcttatgcagaactcagacgcagaaacctacacaagaagtatag
gtacaagagtagaatcaaagagttagaagcttcattaagatggatggtagatgtggatgttcaagtcaaccaat
ggctagatatcaaagaactcgtggctaagatgtctgaaatgaacacaaaactcgacaagatcacgagccaacca
actgatggtagttgtttcaagagcaatgatagcatatcaccagtgttatcacaaagtagtaggatcgaagcaac
agacggatcttcagaggaagatgaagaagaaagctcaagtaatggatccgaaccaaggatcgatatccacctgc
gatggagttcaagaaaaggaagaaaagatcgtgagatccgattcacggccaagtga

Sequence Listing 12: Predicted BrHR2 Protein Sequence (216 aa)

Mpigevivgaalgitlqvlhqaiikakdrssttkcilvrldatisritplvvhvdkiskrvedserkvieelkr
llekavslveayaelrrrnlhkkyryksrikeleaslrwmvdvdvqvnqwldikelvakmsemntkldkitsqp
tdgscfksndsispvlsqssrieatdgsseedeeesssngsepridihlrwssrkgrkdreirftak

Sequence Listing 13: BrHR3 Genomic Sequence (746 bp)

Atgccgattggtgaggttcttgtaggggctgctcttggaattacactccaagtgcttcatgaagccatcataaa
agcaaaacatagatctttaaccacaaaatgtatcttggaccgcctcgatgctacaatctccaggatcactccgt
tggtggttcatgtcgataagatcagcaaaggggtagaagattctcagaggaaagtcattgaagacctcaagcgt
cttcttgaaaaggctgtttttcttgttgaggcttatgcagaactcagacgcagaaacctactcaagaagtttag
gtatgtatagtttatatagtacatgaaatgcttgaaaagtctttgtgattcttaaaatgtttttgttttgttta
tataatatatatgtgtgtgttgttgatatctaggtacaagagtagaatcaaagagttggaagcttctttaagat
ggatggtagaggtggatgttcaagtcaaccaatggttggatatcaaacaactcctggccaagatgtttgaaatg
aacactaaactcgagaggatcacgtgcccaccaactgattgtaattgtttcaagagaaatgatagcacatcacc
agtgatatcacaaagtagtaatcaaaatatactcgaagcaacagacggatcgtcagaggaagacgaagaagaaa
gcccaaggattgatatccaccttcgatggagttcaagaaaaggagctaaagatcgtgagatccgattcatggtc
aagtga

Sequence Listing 14: Predicted BrHR3 cDNA Sequence (639 bp)

Atgccgattggtgaggttcttgtaggggctgctcttggaattacactccaagtgcttcatgaagccatcataaa
agcaaaacatagatctttaaccacaaaatgtatcttggaccgcctcgatgctacaatctccaggatcactccgt
tggtggttcatgtcgataagatcagcaaaggggtagaagattctcagaggaaagtcattgaagacctcaagcgt
cttcttgaaaaggctgtttttcttgttgaggcttatgcagaactcagacgcagaaacctactcaagaagtttag
gtacaagagtagaatcaaagagttggaagcttctttaagatggatggtagaggtggatgttcaagtcaaccaat
ggttggatatcaaacaactcctggccaagatgtttgaaatgaacactaaactcgagaggatcacgtgcccacca
actgattgtaattgtttcaagagaaatgatagcacatcaccagtgatatcacaaagtagtaatcaaaatatact
cgaagcaacagacggatcgtcagaggaagacgaagaagaaagcccaaggattgatatccaccttcgatggagtt
caagaaaaggagctaaagatcgtgagatccgattcatggtcaagtga

Sequence Listing 15: Predicted BrHR3 Protein Sequence (213 aa)

mpigevlvgaalgitlqvlheaiikakhrslttkcildrldatisritplvvhvdkiskgvedsqrkviedlkr
llekavflveayaelrrrnllkkfryksrikeleaslrwmvevdvqvnqwldikqllakmfemntkleritcpp
tdcncfkrndstspvisqssnqnileatdgsseedeeespridihlrwssrkgakdreirfmvk

1. An isolated nucleic acid molecule which nucleic acid consists essentially of an RPW nucleotide sequence encoding an RPW resistance polypeptide having an N-terminal transmembrane domain and a coiled coil domain and which is capable of recognising and activating in a plant into which said nucleic acid is introduced a specific defense response to challenge with a powdery mildew pathogen which is any of: E. cichoracearum, E. cruciferarum, E. orontii, Oidium lycopersici.

2. A nucleic acid as claimed in claim 1 wherein the RPW nucleotide sequence is derived from an RPW7 or RP8 locus in a plant.

3. An isolated nucleic acid molecule which consists essentially of an RPW nucleotide sequence which:
(i) encodes an RPW resistance polypeptide selected from any shown in Sequence listing 4 (RPW8.1) or Sequence listing 6 (RPW8.2) as Ms-0, Wa-1, Kas-1, or C24; or shown in Sequence listing 9 (BrHR1), 12 (BrHR2), or 15 (BrHR3), or in Example 4 (hr1, hr2, or hr3), or,
(ii) encodes a homologous variant of the RPW resistance polypeptide of (i), which shares at least about 50%, 60%, 70%, 80% or 90% identity therewith,
and wherein the nucleic acid encoding said homologous variant hybridises at 37° C. in a formamide concentration of about 20% and a salt concentration of about 5×SSC with any complement RPW nucleic acid having a sequence selected from: RPW8.1 genomic sequence (shown as 13878 . . . 14719 in Sequence Listing 2); RPW8.2 genomic sequence (shown as 15904 . . . 16829 in Sequence Listing 2); RPW8.1 cDNA sequence or RPW8.2 cDNA sequence complementary to that shown in Sequence listing 4 or Sequence listing 6 as Ms-0, Wa-1, Kas-1, or C24; BrHR1 genomic or cDNA sequence complementary to that shown in Sequence listing 7 or 8, BrHR2 genomic or cDNA sequence complementary to that shown in Sequence listing 10 or 11, BrHR3 genomic or cDNA sequence complementary to that shown in Sequence listing 13 or 14, HR1 genomic sequence (shown as 19087 . . . 20103 in Sequence Listing 1); HR2 genomic sequence (shown as 20600 . . . 21408 in Sequence Listing 1); HR3 genomic sequence (shown as 25912 . . . 26632 in Sequence Listing 1).

4. A nucleic acid as claimed in claim 3 wherein the RPW nucleotide sequence encodes an RPW resistance polypeptide selected RPW8.1 or RPW8.2 sequences which are shown in Sequence Listing 2.

5. A nucleic acid as claimed in any one of claims 1 to 3 wherein the RPW nucleotide sequence is selected from a list consisting of: RPW8.1 genomic sequence (shown as 13878 . . . 14719 complement in Sequence Listing 2); RPW8.2 genomic sequence (shown as 15904 . . . 16829 complement in Sequence Listing 2); RPW8.1 cDNA sequence or RPW8.2 cDNA sequence shown in Sequence listing 4 or Sequence listing 6 as Ms-0, Wa-1, Kas-1, or C24; BrHR1 genomic or cDNA sequence shown in Sequence listing 7 or 8, BrHR2 genomic or cDNA sequence shown in Sequence listing 10 or 11, BrHR3 genomic or cDNA sequence shown in Sequence listing 13 or 14, HR1 genomic sequence (shown as 19087 . . . 20103 complement in Sequence Listing 1); HR2 genomic sequence (shown as 20600 . . . 21408 complement in Sequence Listing 1); HR3 genomic sequence (shown as 25912 . . . 26632 complement in Sequence Listing 1).

6. A nucleic acid as claimed in claim 3 wherein the RPW nucleotide sequence encodes a derivative of an RPW resistance polypeptide of claim 3 (i) by way of addition, insertion, deletion or substitution of one or more amino acids.

7. A nucleic acid as claimed in claim 6 which wherein the encoded derivative comprises the sequence DIKEIKAKISE.

8. A nucleic acid as claimed in claim 3 wherein the RPW nucleotide sequence consists of an allelic, paralogous or orthologous variant of an RPW nucleotide sequence of claim 5.

9. A nucleic acid as claimed in claim 3 wherein the variant is obtainable from a plant selected from: barley; Brassica napus; B. oleracea.

10. An isolated nucleic acid which consists essentially of a nucleotide sequence which is the complement of the RPW nucleotide sequence of any one of the preceding claims.

11. An isolated nucleic acid for use as a probe or primer, said nucleic acid consisting of a distinctive sequence of at least about 16-30 nucleotides in length, which sequence is (i) conserved between
two or more cDNA nucleotide sequences of sequence listing 3 or sequence listing 5; (ii) a sequence degeneratively equivalent to said conserved sequence, or (iii) the complement sequence of either.

12. A nucleic acid primer as claimed in claim 11 which encodes all or part of any one the following conserved amino acid motifs: DIKEIKAKISE; MIAEVAAGGA LGLALSV; RLKLLLENAV SLVEENAELR RRNVRKKFRY MRDIKEFEAK; VDVQ VNQLADIKEL KAKMSEISTK LDK.

13. A nucleic acid primer for amplification of RPW8.1 selected from:
GACCCGTACAGTACTAAGTCTA
GATTTCCGAAATTGATTACAAGAA
ATGCCGATTGGTGAGCTTGCGATA
TCAAGCTCTTATTTTACTACAAGC
AATGGACACTAAACTTGCTGAAGT
CCACAACTATTATGCTTCT
GAACCAAAAACGGCTCGATACTAA
CCGGAATTCATGCCGATTGGTGAGCTTGCGATA
CGCGGATCCTCAAGCTCTTATTTTACTACAAGC
or for amplification of RPW8.2 selected from:
AACTCTTCACCTCGAGAGCTAACA
AGTCGTTTGACACAATTGGGACAT
ATGATTGCTGAGGTTGCCGCA
TCAAGAATCATCACTGCAGAACGT
GCTAAATTACGATGGGTGGTAGAT
CGATGGGTGGTAGATGTGGATGTT
GGATCGCACGGTTTGT
CTGAACTTCTTGCGTACGTTTCT.
CCGGAATTCATGATTGCTGAGGTTGCCGCA
CCGGGATCCTCAAGAATCATCACTGCAGAACGT

14. A method for identifying, cloning, or determining the presence within a plant of a nucleic acid as claimed in any one of claims 1 to 9, which method employs a nucleic acid as claimed in any one of claims 10 to 13.

15. A method as claimed in claim 14, which method comprises the steps of:
(a) providing a preparation of nucleic acid from a plant cell;
(b) providing a nucleic acid molecule which is a nucleic acid as claimed in claim 10,
(c) contacting nucleic acid in said preparation with said nucleic acid molecule under conditions for hybridisation, and,
(d) identifying nucleic acid in said preparation which hybridises with said nucleic acid molecule.

16. A method as claimed in claim 14, which method comprises the steps of:
(a) providing a preparation of nucleic acid from a plant cell;
(b) providing a pair of nucleic acid molecule primers suitable for PCR, at least one of said primers being a primer of any one of claims 11 to 13,
(c) contacting nucleic acid in said preparation with said primers under conditions for performance of PCR,
(d) performing PCR and determining the presence or absence, and optionally the sequence, of an amplified PCR product.

17. A recombinant vector which comprises the nucleic acid of any one of claims 1 to 9.

18. A vector as claimed in claim 17 wherein the nucleic acid is operably linked to a promoter for transcription in a host cell, wherein the promoter is optionally an inducible promoter.

19. A vector as claimed in claim 17 or claim 18 which is a plant vector.

20. A vector as claimed in claim 19 which is the SE7.5 construct shown in FIG. 3 herein.

21. A method which comprises the step of introducing the vector of any one of claims 17 to 20 into a host cell, and optionally causing or allowing recombination between the vector and the host cell genome such as to transform the host cell.

22. A host cell containing or transformed with a heterologous vector of any one of claims 17 to 20.

23. A method for producing a transgenic plant, which method comprises the steps of:
(a) performing a method as claimed in claim 22 wherein the host cell is a plant cell,
(b) regenerating a plant from the transformed plant cell.

24. A transgenic plant which is optionally selected from a species which is susceptible to powdery mildew, and which is obtainable by the method of claim 23, or which is a clone, or selfed or hybrid progeny or other descendant of said transgenic plant, which in each case includes a heterologous nucleic acid of any one of claims 1 to 9.

25. A transgenic plant as claimed in claim 24 which is selected from: wheat; barley; tomato; Nicotiana spp.

26. A part of propagule from a plant as claimed in claim 24 or claim 25, and which in either case includes a heterologous nucleic acid of any one of claims 1 to 9.

27. An isolated polypeptide which is encoded by the RPW nucleotide sequence of any one of claims 1 to 9.

28. A polypeptide as claimed in claim 27 which is an RPW resistance polypeptide selected from any shown in Sequence listing 4 (RPW8.1) or Sequence listing 6 (RPW8.2) as Ms-0, Wa-1, Kas-1, or C24; or shown in Sequence listing 9 (BrHR1), 12 (BrHR2), or 15 (BrHR3), or in Example 4 (hr1, hr2, or hr3).

29. A method of making the polypeptide of claim 27 or claim 26, which method comprises the step of causing or allowing expression from a nucleic acid of any one of claims 1 to 9 in a suitable host cell.

30. A polypeptide which comprises the antigen-binding site of an antibody having specific binding affinity for the polypeptide of claim 28.

31. A method for influencing or affecting the degree of resistance of a plant to a powdery mildew caused by any one of E. cichoracearurn, E.cruciferarum, E.orontii, Oidium lycopersici, which method comprises the step of causing or allowing expression of a heterologous nucleic acid as claimed in any one of claims 1 to 10 within the cells of the plant, following an earlier step of introducing the nucleic acid into a cell of the plant or an ancestor thereof.

32. A method as claimed in claim 31 for increasing a plant's powdery mildew disease resistance, wherein the nucleic acid is a nucleic acid as claimed in any one of claims 1 to 9.

33. An isolated nucleic acid molecule encoding the promoter of an RPW nucleotide sequence of claim 5, or a homologous variant thereof which has promoter activity which is operably linked to a heterologous coding sequence.

34. A nucleic acid as claimed in claim 33 wherein the promoter is wound and SA induced but not JA induced.

35. A nucleic acid as claimed in claim 33 wherein the promoter is that of RPW8.1 (15904 to 14719) or RPW8.2 (16829 to 19087) of Sequence Listing 1.
